SEQUENCE LISTING (2 sequences)

SEQ ID NO: 1 - RNA, 4284 nt, synthetic construct
  source 1..4284: mol_type = other RNA
gagaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 2 - RNA, 4001 nt, synthetic construct
  source 1..4001: mol_type = other RNA
gggaaataagagagaaaagaagagtaagaagaaatataagaccccggcgccgccaccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtagagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggactgcgccctggaccctctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtcttcaccaacgtgtacgccgacagttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagaagagcaccaacctggtgaagaacaagtgcgtgaacttcaacttcaacggccttaccggcaccggcgtgctgaccgagagcaacaagaaattcctgccctttcagcagttcggccgggacatcgccgacaccaccgacgctgtgcgggatccccagaccctggagatcctggacatcaccccttgcagcttcggcggcgtgagcgtgatcaccccaggcaccaacaccagcaaccaggtggccgtgctgtaccaggacgtgaactgcaccgaggtgcccgtggccatccacgccgaccagctgacacccacctggcgggtctacagcaccggcagcaacgtgttccagacccgggccggttgcctgatcggcgccgagcacgtgaacaacagctacgagtgcgacatccccatcggcgccggcatctgtgccagctaccagacccagaccaattcaccccggagggcaaggagcgtggccagccagagcatcatcgcctacaccatgagcctgggcgccgagaacagcgtggcctacagcaacaacagcatcgccatccccaccaacttcaccatcagcgtgaccaccgagattctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagacccctcccatcaaggacttcggcggcttcaattcagccagatcctgcccgaccccagcaagcccagcaagcggagcttcatcgaggacctgctgttcaacaaggtgaccctagccgacgccggcttcatcaagcagtacggcgactgcctcggcgacatagccgcccgggacctgatctgcgcccagaagttcaacggcctgaccgtgctgcctcccctgctgaccgacgagatgatcgcccagtacaccagcgcctgttagccggaaccatcaccagcggctggactttcggcgctggagccgctctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgctagcgccctgggcaagctgcaggacgtggtgaaccagaacgcccaggccctgaacaccctggtgaagcagctgagcagcaacttcggcgccatcagcagcgtgctgaacgacatcctgagccggctggaccctcccgaggccgaggtgcagatcgaccggctgatcactggccggctgcagagcctgcagacctacgtgacccagcagctgatccgggccgccgagattcgggccagcgccaacctggccgccaccaagatgagcgagtgcgtgctgggccagagcaagcgggtggacttctgcggcaagggctaccacctgatgagctttccccagagcgcaccccacggagtggtgttcctgcacgtgacctacgtgcccgcccaggagaagaacttcaccaccgccccagccatctgccacgacggcaaggcccactttccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgatcccctgcagcccgagctggacagcttcaaggaggagctggacaagtacttcaagaatcacaccagccccgacgtggacctgggcgacatcagcggcatcaacgccagcgtggtgaacatccagaaggagatcgatcggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgttgcagctgcggcagctgctgcaagttcgacgaggacgacagcgagcccgtgctgaagggcgtgaagctgcactacacctgataataggctggagcctcggtggcctagcttcttgccccttgggcctccccccagcccctcctccccttcctgcacccgtacccccgtggtctttgaataaagtctgagtgggcggcaaaaaaaaa

(19) United States
(12) Patent Application Publication
(10) Pub. No.: US 2026/0130987 A1
(43) Pub. Date: May 14, 2026

(54) VACCINATION AGAINST PNEUMOCCOCAL AND COVID-19 INFECTIONS

(71) Applicant: Pfizer Inc., New York, NY (US)

(72) Inventors: Alejandro David Cane, Berwyn, PA (US); William Carl Gruber, Saline, MI (US); Kathrin Ute Jansen, New York, NY (US); Luis Pascual Jodar Martin-Montalvo, Miami, FL (US); Stephen Paul Lockhart, Hurst, Reading (GB); Daniel Alfred Scott, Castle Rock, CO (US); Wendy Jo Watson, Blue Bell, PA (US); Kari Ann Yacisin, Albuquerque, NM (US)

(73) Assignee: Pfizer Inc., New York, NY (US)

(21) Appl. No.: 19/121,502

(22) PCT Filed: Oct. 17, 2023
(86) PCT No.: PCT/IB2023/060483

§ 371 Date: Apr. 16, 2025

Related U.S. Application Data
(60) Provisional application No. 63/417,634, filed on Oct. 19, 2022.
(60) Provisional application No. 63/479,173, filed on Jan. 9, 2023.

(51) Int. Cl. A61K 39/215; A61K 39/00; A61K 39/09; A61K 39/385; A61P 31/04; A61P 31/14; C12N 7/00

(52) U.S. Cl. CPC A61K 39/215; A61K 39/092; A61K 39/385; A61P 31/04; A61P 31/14; C12N 7/00; A61K 2039/53; A61K 2039/575; A61K 2039/6037; A61K 2039/70

(57) ABSTRACT

The invention relates to vaccination of human subjects, in particular elderly, against pneumoccocal and COVID-19 infections.

### CROSS-REFERENCE TO RELATED APPLICATIONS

The present application claims the benefit of U.S. Provisional Patent Application Prov 1 63/417,634, filed on Oct. 19, 2022, and U.S. Provisional Patent Application 63/479,173, filed on Jan. 9, 2023. The entire contents of the aforementioned applications are herein incorporated by reference in their entireties. The entire contents of International Application PCT/IB2022/053951, filed Apr. 28, 2022, now published as WO2022234405 on Nov. 10, 2022, are herein incorporated by reference in its entirety.

### FIELD OF THE INVENTION

The present disclosure relates to a combination of treatment and/or prophylaxis for humans, in particular elderly, adolescent, and infant subjects, wherein the combination comprises any one of vaccines comprising proteins, vaccines comprising polysaccharides, vaccines comprising polysaccharide-protein conjugates, and antiviral heterocyclic compounds, and combinations thereof, in further combination with mRNA vaccines. In particular, the present disclosure relates to any of the following combinations: a vaccine comprising a protein and an mRNA vaccine; a vaccine comprising a polysaccharide and an mRNA vaccine; a vaccine comprising a polysaccharide-protein conjugate and an mRNA vaccine; an antiviral heterocyclic compound and an mRNA vaccine; a vaccine comprising a protein, an antiviral heterocyclic compound, and an mRNA vaccine; a vaccine comprising a polysaccharide, an antiviral heterocyclic compound, and an mRNA vaccine; a vaccine comprising a polysaccharide-protein conjugate, an antiviral heterocyclic compound, and an mRNA vaccine; an antiviral heterocyclic compound and an mRNA vaccine; and combinations thereof, wherein the treatment and/or prophylaxis is directed against bacterial, viral, and/or COVID-19 infections. In some embodiments, the invention discloses and relates to vaccination of human subjects, in particular elderly subjects, with pneumoccocal conjugate vaccines in combination with mRNA vaccines. In particular, the present invention relates to vaccinations against pneumoccocal and COVID-19 infections.

### BACKGROUND OF THE INVENTION

Protein and/or polysaccharide antigens from pathogens have long been used in vaccines, designed to elicit neutralizing antibody and/or cell-mediated immune responses in the recipient, specific for the antigen. Cell-mediated immune responses, particularly the generation of effector T-cells (including cytotoxic T-cells), may be a desirable component of the immune response elicited from vaccines having a polypeptide and/or polysaccharide component. Antibodies may also be a desirable component of the protective immune response for pathogens particularly bacteria and certain viruses such as the influenza viruses.

Nucleic acid-based vaccines may elicit cell-mediated immunity (e.g., involving effector T-cells, such as interferon-g secreting antigen-specific T-cells and antigen-specific cytotoxic T-cells). Generating antibodies against the antigen that is encoded and expressed by the nucleic acid component may also be a desirable component of the immune response elicited from nucleic acid-based vaccines.

There is a need to improve treatment and/or immunization regimens such that immune responses observed with coadministration of a composition, such as a first immunogenic composition having a polypeptide and/or polysaccharide component, or a first composition comprising an antiviral heterocyclic compound; and a second immunogenic composition having a nucleic acid component, wherein the concomitant administration of the first composition and the second composition is non-inferior, or preferably enhanced, for the respective antigens compared to the effect(s) and/or immune response against the respective antigens when the compositions are not co-administered. For example, the first immunogenic composition may include a polypeptide, a toxoid, an antiviral heterocyclic compound, a polysaccharide, and/or a polysaccharide-conjugate. The compositions may be useful for generating an immune response, for example, to reduce the likelihood of infection, by an infectious agent, such as pneumococci.

Infections caused by pneumococci are a major cause of morbidity and mortality all over the world. Pneumonia, febrile bacteraemia and meningitis are the most common manifestations of invasive pneumococcal disease, whereas bacterial spread within the respiratory tract may result in middle-ear infection, sinusitis or recurrent bronchitis. Compared with invasive disease, the non-invasive manifestations are usually less severe, but considerably more common.

In Europe and the United States, pneumococcal pneumonia is the most common community-acquired bacterial pneumonia, estimated to affect approximately 100 per 100,000 adults each year. The corresponding figures for febrile bacteraemia and meningitis are 15-19 per 100 000 and 1-2 per 100,000, respectively. The risk for one or more of these manifestations is much higher in infants and elderly people, as well as immune compromised persons of any age. Even in economically developed regions, invasive pneumococcal disease carries high mortality; for adults with pneumococcal pneumonia the mortality rate averages 10%-20%, whilst it may exceed 50% in the high-risk groups. Pneumonia is by far the most common cause of pneumococcal death worldwide.

Pneumococcal conjugate vaccines (PCVs) are pneumococcal vaccines used to protect against disease caused by S. pneumoniae (pneumococcus). There are currently three PCV vaccines available on the global market: PREVNAR® (PREVENAR® in some countries) (heptavalent vaccine), SYNFLORIX® (a decavalent vaccine) and PREVNAR 13® (PREVENAR 13® in some countries) (tridecavalent vaccine).

Severe acute respiratory syndrome coronavirus 2 (SARS-COV-2) is the virus that causes COVID-19 (coronavirus disease 2019), the respiratory illness responsible for the COVID-19 pandemic. SARS-COV-2 is a positive-sense single-stranded RNA virus. The virus primarily spreads between people through close contact and via respiratory droplets produced from coughs or sneezes. It mainly enters human cells by binding to the angiotensin converting enzyme 2 (ACE2).

There is a need to effectively protect patients against both pneumococcal infections and/or pneumococcal disease as well as against coronavirus disease.

An object of the schedules of administration of the present invention is to provide for appropriate protection against S. pneumoniae and COVID-19.

### SUMMARY OF THE INVENTION

In one aspect the disclosure relates to a method for eliciting an immunoprotective response in a human subject against an infectious disease-causing bacterium (e.g., selected from any one of S. pneumoniae, N. meningitidis, C. difficile, and E. coli) and betacoronavirus (e.g., SARS-COV-2), the method includes co-administering to the human subject an effective dose of a first immunogenic composition including an antigen selected from any one of a polypeptide, toxoid, polysaccharide, and polysaccharide conjugate; and a second immunogenic composition including mRNA against a betacoronavirus. In an aspect, said first immunogenic composition against the bacterium and said second immunogenic composition mRNA vaccine against betacoronavirus are co-administered, e.g., concurrently or concomitantly. In a preferred embodiment, the antigen selected from any one of a polypeptide, toxoid, polysaccharide, and polysaccharide conjugate, is derived from the infectious disease-causing bacterium.

In an aspect the invention is directed to a method for eliciting an immunoprotective response in a human subject against S. pneumoniae and Severe Acute Respiratory Syndrome Coronavirus 2 (SARS-COV-2), the method comprising co-administering to the human subject an effective dose of a pneumococcal conjugate vaccine and of an mRNA vaccine against SARS-COV-2. In an aspect, said pneumococcal conjugate vaccine and said mRNA vaccine against SARS-COV-2 are administered concurrently or concomitantly.

The invention further relates to a pneumococcal conjugate vaccine and an mRNA vaccine against SARS-COV-2 for use in a method for eliciting an immunoprotective response in a human subject against S. pneumoniae and Severe Acute Respiratory Syndrome Coronavirus 2 (SARS-COV-2), said method comprising co-administering to the human subject said vaccines. Another aspect of the invention relates to the use of the co-administration of a pneumococcal conjugate vaccine and of an mRNA vaccine against SARS-COV-2 as a booster dose of an mRNA vaccine against SARS-COV-2.

The invention further relates to the co-administration of a pneumococcal conjugate vaccine and of an mRNA vaccine against SARS-COV-2 for use as a booster dose of an mRNA vaccine against SARS-COV-2 and to the co-administration of a pneumococcal conjugate vaccine and of an mRNA vaccine against SARS-COV-2 for use in a method of boostering an mRNA vaccine against SARS-COV-2.

In one aspect the disclosure relates to a method for eliciting an immunoprotective response in a human subject against an infectious disease-causing respiratory virus (e.g., RSV) and betacoronavirus (e.g., SARS-COV-2), the method includes co-administering to the human subject an effective dose of a first immunogenic composition including an antigen selected from any one of a polypeptide, toxoid, polysaccharide, and polysaccharide conjugate; and a second immunogenic composition including mRNA against a betacoronavirus. In an aspect, said first immunogenic composition against the bacterium and said second immunogenic composition mRNA vaccine against betacoronavirus are co-administered, e.g., concurrently or concomitantly. In a preferred embodiment, the antigen selected from any one of a polypeptide, toxoid, polysaccharide, and polysaccharide conjugate, is derived from the infectious disease-causing bacterium.

In one aspect, the present disclosure relates to concomitant uses of compounds of Formula (I),

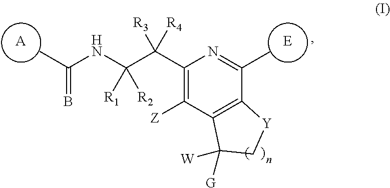

or pharmaceutically acceptable salts, esters, or prodrugs thereof, which inhibit Human Respiratory Syncytial Virus (HRSV or RSV) or Human Metapneumovirus (HMPV) inhibitors, with any of the compositions described herein. The present invention further relates to pharmaceutical compositions comprising the aforementioned compounds for administration to a subject suffering from HRSV or HMPV infection. The disclosure also relates to methods of treating an HRSV or HMPV infection in a subject by administering a pharmaceutical composition comprising the compounds of the present disclosure, which are described in U.S. Pat. No. 11,572,367, filed Jul. 16, 2020, and published on Feb. 7, 2023. For example, in some embodiments, the antiviral compound is a compound represented by Formula (I) shown above, or a pharmaceutically acceptable salt thereof, wherein: A is selected from the group consisting of: 1) optionally substituted aryl; and 2) optionally substituted heteroaryl; B is O or S; R1 and R2 are each independently selected from the group consisting of: 1) hydrogen; 2) fluorine; and 3) optionally substituted —C1-C6 alkyl; alternatively, R1 and R2 are taken together with the carbon atom to which they are attached to form an optionally substituted 3- to 6-membered ring; Z is selected from the group consisting of: 1) hydrogen; 2) halogen; 3) hydroxy; 4) cyano; 5) nitro; 6) optionally substituted —C1-C6 alkoxy; and 7) optionally substituted —C1-C6 alkyl; W is selected from the group consisting of: 1) hydrogen; 2) optionally substituted —C1-C6 alkoxy; 3) optionally substituted —C1-C6 alkyl; and 4) optionally substituted —C3-C6 cycloalkyl; G is selected from the group consisting of: 1) —C(O)OR12; 2) —C(O)NR11R12; 3) optionally substituted —C1-C6 alkyl-CN; 4) optionally substituted —C1-C6 alkyl-C(O)NR11R12; 5) optionally substituted —C1-C6 alkyl-C(O)NR11S(O)2R12; 6) optionally substituted —C1-C6 alkyl-OC(O)NR11R12; 7) optionally substituted —C1-C6 alkyl-NHR13; 8) optionally substituted —C1-C6 alkyl-NHC(O)R13; and 9) —C(O)NR11S(O)2R12; n is 1, 2 or 3; Y is O, S, S(O)2, or NR14; E is selected from the group consisting of: 1) optionally substituted aryl; 2) optionally substituted heteroaryl; 3) optionally substituted 3- to 8-membered heterocyclic, and 4) optionally substituted alkynyl; R3 is hydroxy or fluorine; R4 is selected from the group consisting of: 1) hydrogen; 2) optionally substituted —C1-C6 alkyl; 3) optionally substituted —C3-C8 cycloalkyl; and 4) optionally substituted 3- to 8-membered heterocyclic; R11 at each occurrence is independently selected from the group consisting of: 1) hydrogen; 2) optionally substituted —C1-C8-alkyl; 3) optionally substituted-C3-C8-cycloalkyl; 4) optionally substituted 3- to 8-membered heterocyclic; 5) optionally substituted aryl; 6) optionally substituted arylalkyl; 7) optionally substituted heteroaryl; and 8) optionally substituted heteroarylalkyl; R12 at each occurrence is independently selected from the group consisting of: 1) hydrogen; 2) optionally substituted-C1-C8-alkyl; 3) optionally substituted-C3-C8-cycloalkyl; 4) optionally substituted 3- to 8-membered heterocyclic; 5) optionally substituted aryl; 6) optionally substituted arylalkyl; 7) optionally substituted heteroaryl; and 8) optionally substituted heteroarylalkyl; alternatively, Rn and R12 are taken together with the nitrogen atom to which they are attached to form a 3- to 12-membered heterocyclic ring; R13 at each occurrence is independently selected from the group consisting of: 1) Optionally substituted-C1-C8 alkyl; 2) Optionally substituted-C3-C8 cycloalkyl; 3) Optionally substituted 3- to 8-membered heterocyclic; 4) Optionally substituted aryl; 5) Optionally substituted arylalkyl; 6) Optionally substituted heteroaryl; and 7) Optionally substituted heteroarylalkyl; and R14 is selected from: 1) hydrogen; 2) optionally substituted-C1-C8-alkyl; and 3) optionally substituted-C3-C5-cycloalkyl. In some embodiments, the first composition comprises the compound comprising Formula I as described in U.S. Pat. No. 11,572,367, or a pharmaceutically acceptable salt thereof, and a pharmaceutically acceptable carrier, diluent or excipient.

In one aspect, the method of treating and/or elicing an immune response against a viral respiratory infection comprises administering a compound comprising Formula I as described in U.S. Pat. No. 11,572,367 or a pharmaceutically acceptable salt thereof, and any one of the immunogenic compositions described herein, wherein the immunogenic composition comprises mRNA encapsulated in a lipid nanoparticle. In one aspect, the disclosure relates to a method of treating an RSV infection in a human in need thereof, comprising administering to the subject a therapeutically effective amount of a compound comprising Formula I as described in U.S. Pat. No. 11,572,367 or a pharmaceutically acceptable salt thereof, wherein the method further comprises co-administering any of the immunogenic compositions described herein, such as for example, an immunogenic composition comprising an mRNA encoding a betacoronavirus, encapsulated in a lipid nanoparticle. In another aspect, the disclosure relates to a method of treating an RSV infection in a human in need thereof, comprising administering to the subject a therapeutically effective amount of a compound comprising Formula I as described in U.S. Pat. No. 11,572,367 or a pharmaceutically acceptable salt thereof, wherein the method further comprises co-administering any of the immunogenic compositions described herein, such as for example, an immunogenic composition comprising an mRNA encoding a influenza, encapsulated in a lipid nanoparticle. In some embodiments, the method further includes the step of administering to the subject an anti-RSV agent. In some embodiments, the method further includes administering to the subject a steroid anti-inflammatory compound. In some embodiments, the method further includes administering to the subject a therapeutically effective amount of a compound comprising Formula I as described in U.S. Pat. No. 11,572,367 or a pharmaceutically acceptable salt thereof and a therapeutically effective amount of an anti-COVID agent. In some embodiments, the compound and the anti-RSV agent are co-formulated.

17. The method of claim 13, wherein the compound comprising Formula I as described in U.S. Pat. No. 11,572,367 or a pharmaceutically acceptable salt thereof and the anti-COVID agent, such as PAXLOVID® are co-administered. In an aspect the invention is directed to a method for eliciting an immunoprotective response in a human subject against Neisseria meningitidis and betacoronavirus (e.g., SARS-COV-2) and RSV, the method comprising co-administering to the human subject an effective dose of a first immunogenic composition including an antigen derived from Neisseria meningitidis and of an mRNA vaccine against betacoronavirus, further comprising co-administering a compound comprising Formula I as described in U.S. Pat. No. 11,572,367 or a pharmaceutically acceptable salt thereof. In some embodiments, said first immunogenic composition and said mRNA vaccine against betacoronavirus are administered concurrently or concomitantly. In some embodiments, the compound is co-administered, e.g., concurrently or concomitantly, to either the first or the second immunogenic composition. The invention further relates to a first immunogenic composition including an antigen derived from Neisseria meningitidis and an mRNA vaccine against SARS-COV-2 and RSV for use in a method for eliciting an immunoprotective response in a human subject against Neisseria meningitidis and betacoronavirus and RSV, said method comprising co-administering to the human subject said compositions and compounds. Another aspect of the invention relates to the use of the co-administration of a first immunogenic composition including an antigen derived from Neisseria meningitidis and of an mRNA vaccine against betacoronavirus as a booster dose of an mRNA vaccine against the betacoronavirus and further comprising co-administering a compound comprising Formula I as described in U.S. Pat. No. 11,572,367 or a pharmaceutically acceptable salt thereof.

The invention further relates to the co-administration of a first immunogenic composition including an antigen derived from Neisseria meningitidis and of an mRNA vaccine against betacoronavirus for use as a booster dose of an mRNA vaccine against betacoronavirus and to the co-administration of a first immunogenic composition including an antigen derived from Neisseria meningitidis and of an mRNA vaccine against betacoronavirus for use in a method of boostering an mRNA vaccine against betacoronavirus, wherein the method further comprises co-administrating a compound comprising Formula I as described in U.S. Pat. No. 11,572,367 or a pharmaceutically acceptable salt thereof with either the first or second immunogenic composition or both.

In an aspect the invention is directed to a method for eliciting an immunoprotective response in a human subject against Clostridium difficile, now Clostridioides difficile (C. difficile) and betacoronavirus (e.g., SARS-COV-2) and RSV, the method comprising co-administering to the human subject an effective dose of a first immunogenic composition including an antigen derived from C. difficile and of an mRNA vaccine against betacoronavirus. In an aspect, said first immunogenic composition and said mRNA vaccine against betacoronavirus are administered concurrently or concomitantly, wherein the method further comprises co-administrating a compound comprising Formula I as described in U.S. Pat. No. 11,572,367 or a pharmaceutically acceptable salt thereof with either the first or second immunogenic composition or both. The invention further relates to a first immunogenic composition including an antigen derived from C. difficile and an mRNA vaccine against SARS-COV-2 and a further compound comprising Formula I as described in U.S. Pat. No. 11,572,367 or a pharmaceutically acceptable salt thereof for use in a method for eliciting an immunoprotective response in a human subject against C. difficile and betacoronavirus and RSV, said method comprising co-administering to the human subject said compositions.

Another aspect of the invention relates to the use of the co-administration of a first immunogenic composition including an antigen derived from C. difficile and of an mRNA vaccine against betacoronavirus as a booster dose of an mRNA vaccine against the betacoronavirus and a further compound comprising Formula I as described in U.S. Pat. No. 11,572,367 or a pharmaceutically acceptable salt thereof for use in a method of treating RSV.

The invention further relates to the co-administration of a first immunogenic composition including an antigen derived from C. difficile and of an mRNA vaccine against betacoronavirus for use as a booster dose of an mRNA vaccine against betacoronavirus and to the co-administration of a compound comprising Formula I as described in U.S. Pat. No. 11,572,367 or a pharmaceutically acceptable salt thereof for use in a method of treating RSV. In an aspect the invention is directed to a method for eliciting an immunoprotective response in a human subject against Escherichia coli (E. coli) and betacoronavirus (e.g., SARS-COV-2) and RSV, the method comprising co-administering to the human subject an effective dose of a first immunogenic composition including an antigen derived from E. coli and of an mRNA vaccine against betacoronavirus and a compound comprising Formula I as described in U.S. Pat. No. 11,572,367 or a pharmaceutically acceptable salt thereof. In an aspect, said compound and first immunogenic composition and said mRNA vaccine against betacoronavirus are administered concurrently or concomitantly.

The invention further relates to a first immunogenic composition including an antigen derived from E. coli and an mRNA vaccine against SARS-COV-2 for use in a method for eliciting an immunoprotective response in a human subject against E. coli and betacoronavirus and a compound comprising Formula I as described in U.S. Pat. No. 11,572,367 or a pharmaceutically acceptable salt thereof for use in a method of treating RSV, said method comprising co-administering to the human subject said compositions.

Another aspect of the invention relates to the co-administration of a compound comprising Formula I as described in U.S. Pat. No. 11,572,367 or a pharmaceutically acceptable salt thereof and a first immunogenic composition including an antigen derived from E. coli and of an mRNA vaccine against betacoronavirus as a booster dose of an mRNA vaccine against the betacoronavirus.

The invention further relates to the co-administration of a compound comprising Formula I as described in U.S. Pat. No. 11,572,367 or a pharmaceutically acceptable salt thereof for use in a method of treating RSV and a first immunogenic composition including an antigen derived from E. coli and of an mRNA vaccine against betacoronavirus for use as a booster dose of an mRNA vaccine against betacoronavirus and to the co-administration of a first immunogenic composition including an antigen derived from E. coli and of an mRNA vaccine against betacoronavirus for use in a method of boostering an mRNA vaccine against betacoronavirus.Figures

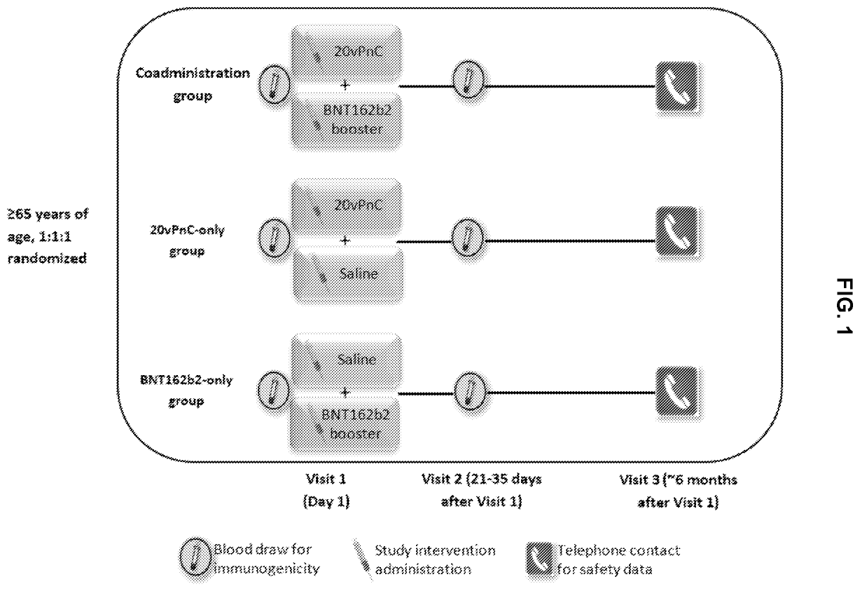

FIG. 1 Schematic representation of the design of a study to describe the safety and immunogenicity of co-administration of a 20-valent Pneumococcal Conjugate Vaccine (20vPnC) when Coadministered with an mRNA vaccine to prevent infection with SARS-COV-2 (BNT162b2), together at the same visit compared to each of the vaccines given alone in adults ≥65 years of age.

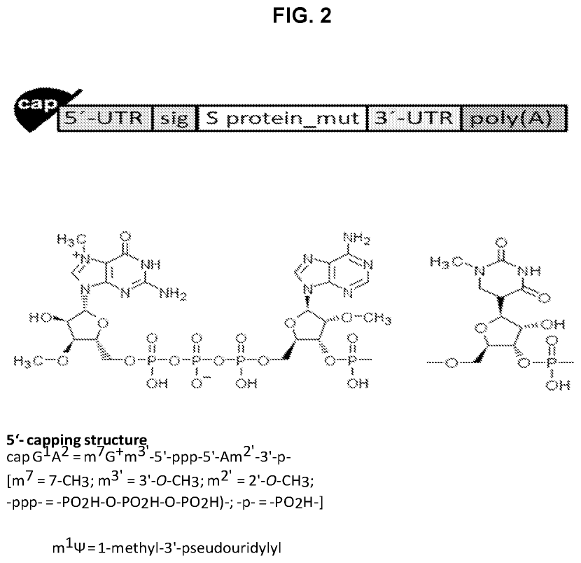

FIG. 2 Schematic and sequence relating to the nucleoside-modified mRNA (modRNA) sequence of the vaccine BNT162b2 (Comirnaty®; INN: tozinameran); Description: Messenger RNA encoding the full-length SARS-COV-2 spike glycoprotein. UTR=Untranslated region; sig=extended signal sequence of the S glycoprotein; S protein_mut=S glycoprotein sequence containing mutations K986P and V987P; poly(A)=polyadenylate signal tail.

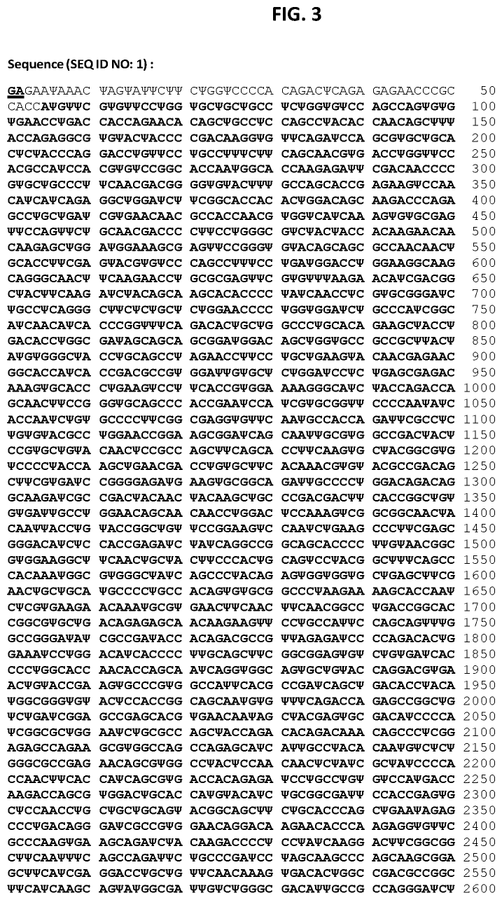

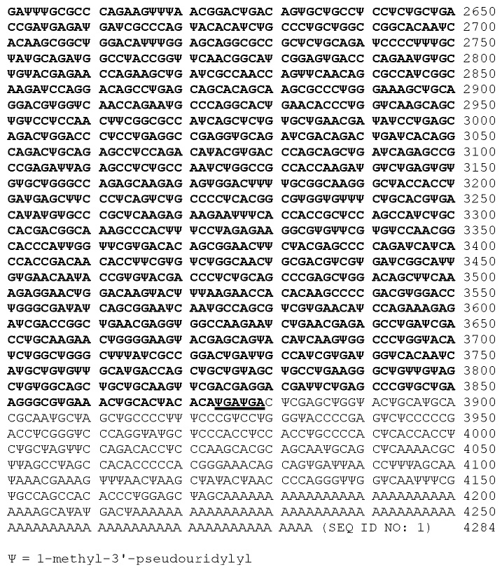

FIG. 3 The putative sequence of the vaccine mRNA-1273 (SEQ ID NO: 2)

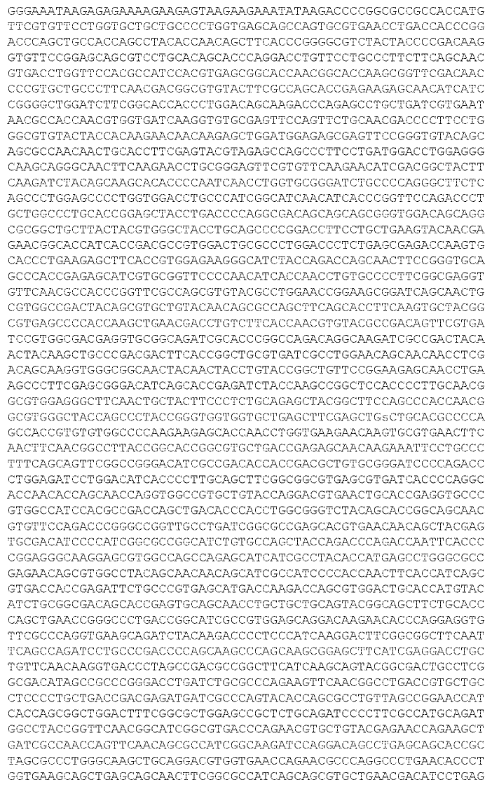

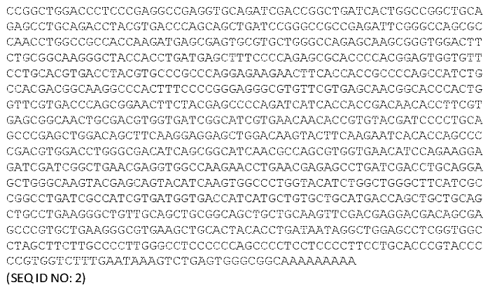

FIG. 4 OPA GMTs (With 2-Sided 95% Cls) 1 Month After Vaccination by Group and Vaccine Serotype; GMTs=geometric mean titres; OPA=opsonophagocytic activity; PCV20=20-valent pneumococcal conjugate vaccine.

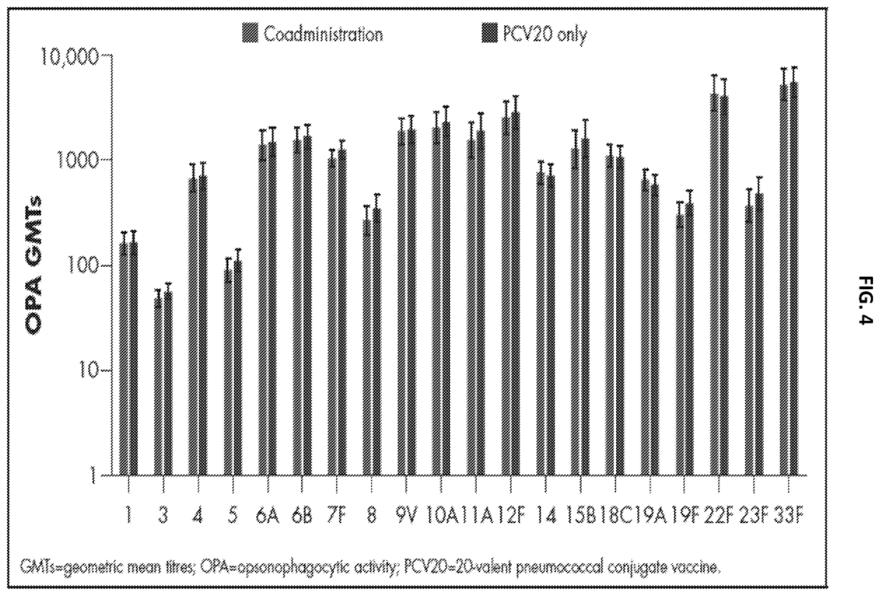

FIG. 5. SARS-COV-2 Full-length S-binding IgG GMCs (With 2-Sided 95% Cls) Before and 1 Month After BNT162b2 by Group; GMCs=geometric mean concentrations.

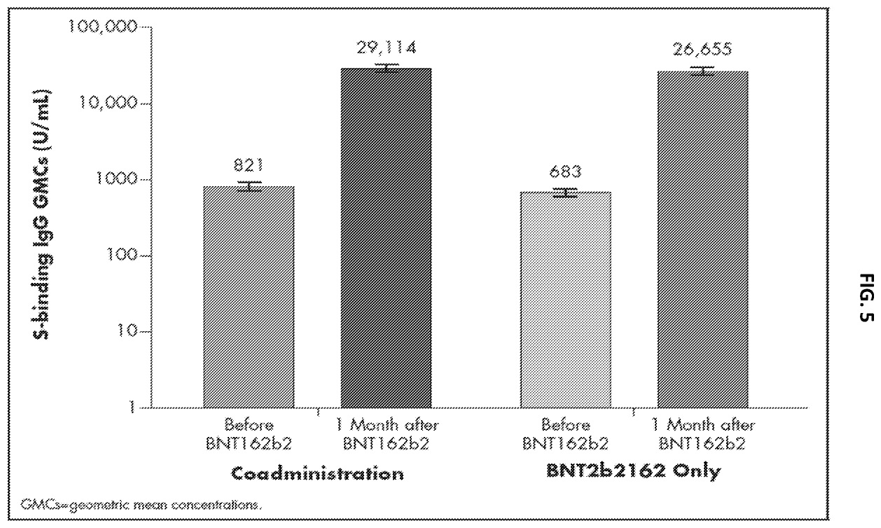

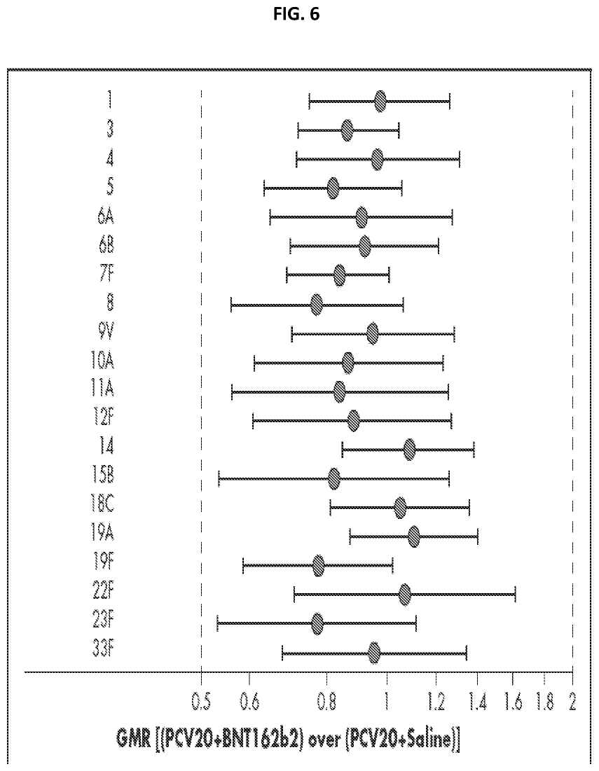

FIG. 6. Model-Based OPA GMRs (With 95% Cls) 1 Month After PCV20 (Evaluable Immunogenicity Population); BLQ=below limit of quantitation; BMI=body mass index; GMR=geometric mean ratio; LLOQ=lower limit of quantitation; LS=least squares; OPA=opsonophagocytic activity. Assay results below the LLOQ or denoted as BLQ were set to 0.5×LLOQ in the analysis. 1 Month after PCV20 refers to 1 month after vaccination with PCV20 for both groups. GMRs (PCV20+BNT162b2 to PCV20+Saline) and 2-sided Cls were calculated by exponentiating the difference of LS means for the OPA titres and the corresponding Cls based on the regression model adjusted with vaccine group, sex, smoking status, age at vaccination in years, baseline log-transformed OPA titres, prior pneumococcal vaccination status, and BMI group.

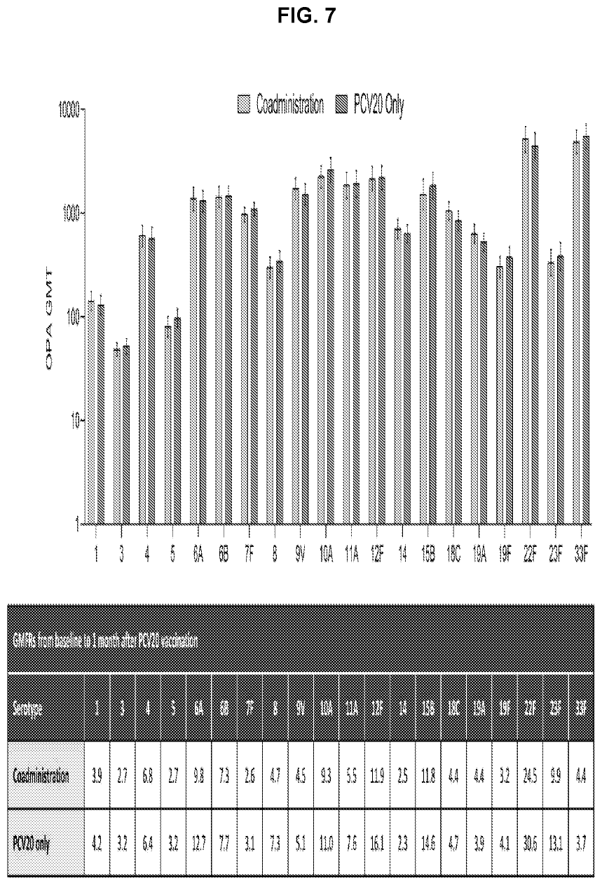

FIG. 7—Immune Responses to PCV20 When Coadministered with a BNT162b2 Booster Dose. When coadministered with BNT162b2, PCV20 elicited robust immune responses at 1 month after vaccination to all 20 serotypes that were similar to that achieved when PCV20 was given alone (FIG. 7). The observed GMFRs from baseline to 1 month after PCV20 were generally similar for most serotypes when PCV20 was coadministered with BNT162b2 (2.5-24.5) or given alone (2.3-30.6). Percentages of participants with a ≥4-fold rise in OPA titres from baseline to 1 month after PCV20 vaccination were generally similar in the Coadministration (24.6-67.9%) and PCV20-only (22.7-71.0%) groups for most serotypes. The proportions of participants with OPA titres ≥LLOQ 1 month after vaccination with PCV20 were also similar (71.5-98.3% and 76.0-99.5% in the Coadministration and PCV20-only groups, respectively). The observed GMFRs from baseline to 1 month after PCV20 were generally similar for most serotypes when PCV20 was coadministered with BNT162b2 (2.5-24.5) or given alone (2.3-30.6). Percentages of participants with a ≥4-fold rise in OPA titres from baseline to 1 month after PCV20 vaccination were generally similar in the Coadministration (24.6-67.9%) and PCV20-only (22.7-71.0%) groups for most serotypes. The proportions of participants with OPA titres ≥LLOQ 1 month after vaccination with PCV20 were also similar (71.5-98.3% and 76.0-99.5% in the Coadministration and PCV20-only groups, respectively).

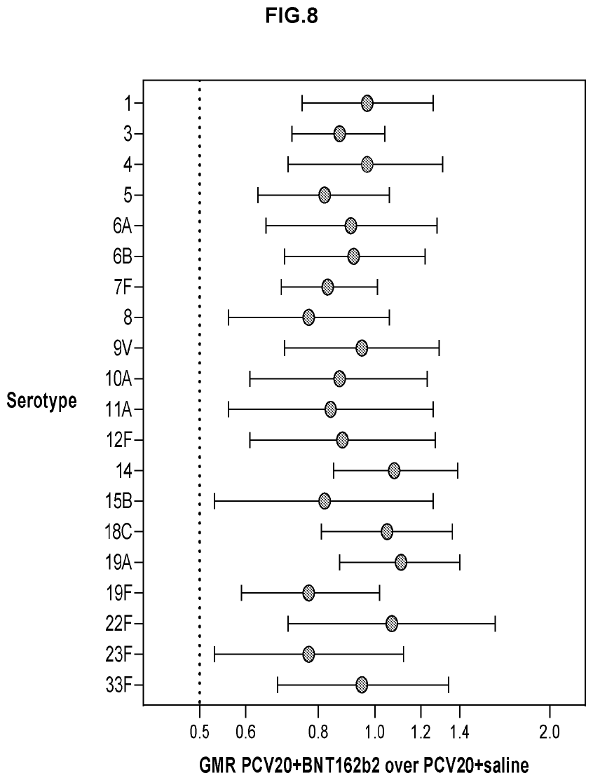

FIG. 8—The post hoc analyses found the model-based OPA geometric mean ratios (GMRs) of the Coadministration group to the PCV20-only group 1 month after PCV20 ranged from 0.77 (serotypes 8, 19F, and 23F) to 1.11 (serotype 19A), with the lower bound of the GMR >0.5 for all 20 serotypes.

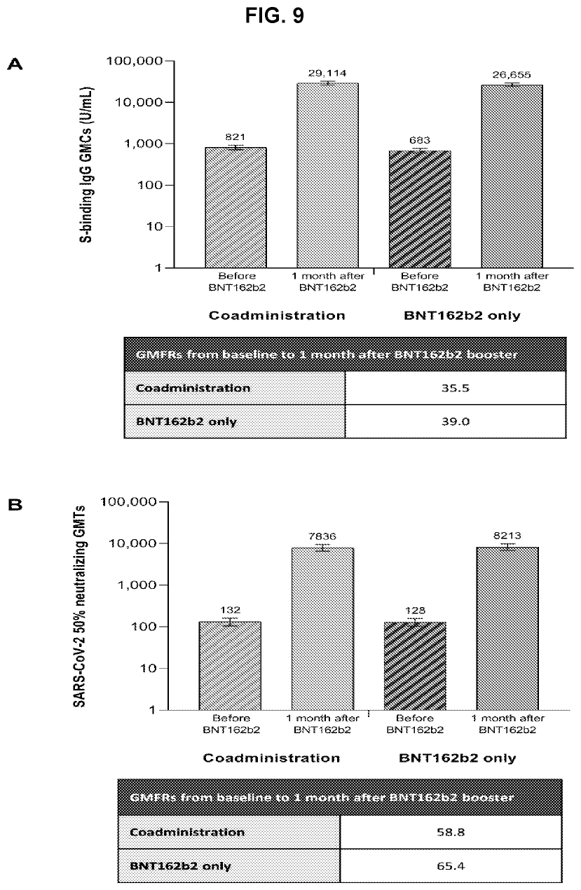

FIG. 9A;FIG. 9B-Immune Responses to BNT162b2 Booster Dose When Coadministered with PCV20. The BNT162b2 booster elicited robust immune IgG responses to the SARS-COV-2 full-length S-binding protein, which were similar whether BNT162b2 was coadministered with PCV20 or given alone (FIG. 9A). Observed GMFRs from before to 1 month after BNT162b2 booster were similar in the Coadministration and BNT162b2 only groups (35.5 and 39.0, respectively). Increases in reference strain neutralizing GMTs observed in the Coadministration and BNT162b2-only groups were also similar (FIG. 9B), with observed GMFRs from before to 1 month after the booster dose of BNT162b2 of 58.8-fold (Coadministration group) and 65.4-fold (BNT162b2 only group).

### DETAILED DESCRIPTION OF THE INVENTION

The present invention combines vaccinations with polysaccharide-protein conjugates, such as PCVs, and mRNA vaccines. The present disclosure further contemplates various combinations of polysaccharide-protein conjugates, such as PCVs, mRNA vaccines, and antiviral heterocyclic compounds.

1. mRNA Vaccines of the Invention

The present invention relates to mRNA vaccines in general. To our best knowledge, the present invention is the first to combine polysaccharide-protein conjugates, such as PCVs, and mRNA vaccines in humans. The present disclosure further contemplates combinations of polysaccharide-protein conjugates, such as PCVs, mRNA vaccines, and antiviral heterocyclic compounds in any combinations thereof.

A number of mRNA vaccine platforms are available in the prior art. The basic structure of in vitro transcribed (IVT) mRNA closely resembles “mature” eukaryotic mRNA, and consists of (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail. The non-coding structural features play important roles in the pharmacology of mRNA and can be individually optimized to modulate the mRNA stability, translation efficiency, and immunogenicity.

By incorporating modified nucleosides, mRNA transcripts referred to as “nucleoside-modified mRNA” can be produced with reduced immunostimulatory activitiy, and therefore an improved safety profile can be obtained. In addition, modified nucleosides allow the design of mRNA vaccines with strongly enhanced stability and translation capacity, as they cab avoid the direct antiviral pathways that are induced by type IFNs and are programmed to degrade and inhibit invading mRNA. For instance, the replacement of uridine with pseudouridine in IVT mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules. Another approach is the design of “self-amplifying mRNA” constructs. These are mostly derived from alphaviruses, and contain an ORF that is replaced by the antigen of interest together with an additional ORF encoding viral replicase. The latter drives the intracellular amplification of mRNA, and can therefore significantly increase the antigen expression capacity.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

Preferably, the mRNA vaccine of the present invention is a vaccine directed against infectious disease, preferably against viral infectious disease, preferably coronavirus disease, preferably against Covid-19 disease. In other embodiments, WO2022234405 (PCT/IB2022/053951), the entire contents of which are herein incorporated by reference in its entirety.

One particularly preferred embodiment of the invention combines a PCV of the invention with the mRNA vaccine BNT162b2 (Comirnaty®).

In another embodiment, the mRNA vaccine includes a sequence having residues 1-102 of SEQ ID NO: 1 (see FIG. 2) and residues 103-4284 of SEQ ID NO: 1, wherein the sequence for the SARS-COV-2 antigen of SEQ ID NO: 1 is replaced with SARS-COV-2 antigen of a variant strain.

Another particularly preferred embodiment of the invention combines a PCV of the invention with the mRNA vaccine “mRNA-1273”.

Further mRNA vaccines directed against Covid-19 disease currently undergoing clinical trials include:
1) MRT5500 Sanofi and Translate Bio Preclinical
2) HGC019 Gennova Biopharmaceuticals and HDT Bio Phase I/II
3) ARCOV Academy of Military Medical Sciences, Suzhou Abogen Biosciences, Walvax Biotechnology Phase I
4) ChulaCoV19 Chula Vaccine Research Centre Phase I
5) PTX-COVID19-B Providence Therapeutics Phase I
6) ARCT-021 (LUNAR-COV19) Duke-NUS/Arcturus Therapeutics Phase II
7) CVnCOV CureVac Phase III

The mRNA vaccines of the invention comprise mRNA and preferably nucleoside-modified mRNA.

mRNA useful in the disclosure typically include a first region of linked nucleosides encoding a polypeptide of interest (e.g., a coding region), a first flanking region located at the 5′-terminus of the first region (e.g., a 5-UTR), a second flanking region located at the 3′-terminus of the first region (e.g., a 3-UTR), at least one 5′-cap region, and a 3′-stabilizing region. In some embodiments, the mRNA of the invention further includes a poly-A region or a Kozak sequence (e.g., in the 5′-UTR). In some cases, mRNA of the invention may contain one or more intronic nucleotide sequences capable of being excised from the polynucleotide. In some embodiments, mRNA of the invention may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

mRNA of the invention may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the invention may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity. mRNA of the invention may include one or more modified (e.g., altered or alternative) nucleobases, nucleosides, nucleotides, or combinations thereof. The mRNA useful in a LNP can include any useful modification or alteration, such as to the nucleobase, the sugar, or the intemucleoside linkage (e.g., to a linking phosphate/to a phosphodiester linkage/to the phosphodiester backbone). In certain embodiments, alterations (e.g., one or more alterations) are present in each of the nucleobase, the sugar, and the intenucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

mRNA of the invention may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation. In some embodiments, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction. In some embodiments, the nucleobase is an alternative uracil. Exemplary nucleobases and nucleosides having an alternative uracil include pseudouridine (w), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uracil (xm5s2U), 1-taurinomethyl-4-thio-pseudouridine, 5-methyl-uracil (m5U, i.e., having the nucleobase deoxythymine), 1-methyl-pseudouridine (mV), 5-methyl-2-thio-uracil (m5s2U), I-methyl-4-thio-pseudouridine (m xj/), 4-thio-1-methyl-pseudouridine, 3-methyl-pseudouridine (m \|/), 2-thio-1-methyl-pseudouridine, 1-methyl-1-deaza-pseudouri dine, 2-thio-I-methyl-1-deaza-pseudouri dine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, NI-methyl-pseudouridine, 3-(3-amino-3-carboxypropyl) uracil (acp U), I-methyl-3-(3-amino-3-carboxypropyl) pseudouridine (acp ψ), 5-(isopentenylaminomethyl) uracil (inm5U), 5-(isopentenylaminomethyl)-2-thio-uracil (inm5s2U), 5,2′-0-dimethyl-uridine (m5Um), 2-thio-2′-0_methyl-uridine (s2Um), 5-methoxycarbonylmethyl-2′-0-methyl-uridine (mem Um), 5-carbamoylmethyl-2′-0-methyl-uridine (ncm5Um), 5-carboxymethylaminomethyl-2′-0-methyl-uridine (cmnm5Um), 3,2′-0-dimethyl-uridine (m Um), and 5-(isopentenylaminomethyl)-2′-0-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocy tidine, 4-thio-1-methy 1-pseudoisocy tidine, 4-thio-1-methyl-1-deaza-pseudoisocytidine, 1-methyl-1-deaza-pseudoisocyti dine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f5Cm), N4,N4,2′-0-trimethyl-cytidine (m42Cm), 1-thio-cytosine, 5-hydroxy-cytosine, 5-(3-azidopropyl)-cytosine, and 5-(2-azidoethyl)-cytosine. In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (m1 A), 2-methyl-adenine (m2A), N6-methyl-adenine (m6A), 2-methylthio-N6-methyl-adenine (ms2m6A), N6-isopentenyl-adenine (16A), 2-methylthio-N6-isopentenyl-adenine (ms2i6A), N6-(cis-hydroxyisopentenyl) adenine (io6A), 2-methylthio-N6-(cis-hydroxyisopentenyl) adenine (ms2i06A), N6-glycinylcarbamoyl-adenine (g6A), N6-threonylcarbamoyl-adenine (16A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-0-dimethyl-adenosine (m6Am), N6,N6,2′-0-trimethyl-adenosine (m62Am), 1,2′-0-dimethyl-adenosine (m1 Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2,N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-0-methyl-guanosine (m2Gm), N2,N2-dimethyl-2′-0-methyl-guanosine (m22Gm), 1-methyl-2′-0-methyl-guanosine (mlGm), N2,7-dimethyl-2′-0-methyl-guanosine (m2,7Gm), 2′-0-methyl-inosine (Im), 1,2′-0-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a] 1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

The mRNA may include a 5 ′-cap structure. The 5′-cap structure of a polynucleotide is involved in nuclear export and increasing polynucleotide stability and binds the mRNA Cap Binding Protein (CBP), which is responsible for

polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5 ′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5 ′end of the polynucleotide may optionally also be 2′-0-methylated. 5 ′-decapping through hydrolysis and cleavage of the guanylate cap structure may target a polynucleotide molecule, such as an mRNA molecule, for degradation. Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5 ′-ppp-5′ cap. Additional alternative guanosine nucleotides may be used such as α-methyl-phosphonate and seleno-phosphate nucleotides. Additional alterations include, but are not limited to, 2′-0-methylation of the ribose sugars of 5′-terminal and/or 5 ′-anteterminal nucleotides of the polynucleotide (as mentioned above) on the 2′-hydroxy group of the sugar. Multiple distinct 5 ′-cap structures can be used to generate the 5′-cap of an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5 ′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5 ′-triphosphate group, wherein one guanosine contains an N7-methyl group as well as a 3′-0-methyl group (i.e., N7, ′-0-dimethyl-guanosine-5′-triphosphate-5 ′-guanosine, m7G-3′mppp-G, which may equivalently be designated 3′ 0-Me-m7G (5′) ppp (5′) G). The 3′-0 atom of the other, unaltered, guanosine becomes linked to the 5 ′-terminal nucleotide of the capped polynucleotide (e.g., an mRNA). The N7- and 3′-0-methylated guanosine provides the terminal moiety of the capped polynucleotide (e.g., mRNA). Another exemplary cap is mCAP, which is similar to ARCA but has a 2′-O-methyl group on guanosine (i.e., N7,2′-O-dimethyl-guanosine-5 ′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G (5) ppp (5 ′) G and a N7-(4-chlorophenoxyethyl)-m3′-OG (5) ppp (5 ′) G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog. While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5 ′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5 ′-cap structures known in the art (or to a wild-type, natural or physiological 5′-cap structure). For example, recombinant Vaccinia Virus Capping Enzyme and recombinant 2′-0-methyltransferase enzyme can create a canonical 5 ′-5′-triphosphate linkage between the 5 ′-terminal nucleotide of a polynucleotide and a guanosine cap nucleotide wherein the cap guanosine contains an N7-methylation and the 5′-terminal nucleotide of the polynucleotide contains a 2′-0-methyl. Such a structure is termed the Capl structure. This cap results in a higher translational-competency, cellular stability, and a reduced activation of cellular pro-inflammatory cytokines, as compared, e.g., to other 5 ′cap analog structures known in the art. Other exemplary cap structures include 7 mG (5″)ppp(5′)N,pN2p (Cap 0), 7 mG (5 ′)ppp(5′)NImpNp (Cap 1), 7 mG (5 ′)-ppp(5′NImpN2mp (Cap 2), and m(7)Gpppm(3)(6,6,2′)Apm(2′)Apm(2′)Cpm(2)(3,2′)Up (Cap 4).

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction. 5 ′-terminal caps may include endogenous caps or cap analogs. A 5′-terminal cap may include a guanosine analog. Useful guanosine analogs include inosine, NI-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5 ′-cap. A modification on the 5′-cap may increase the stability of polynucleotide, increase the half-life of the polynucleotide, and could increase the polynucleotide translational efficiency. The modified 5 ′-cap may include, but is not limited to, one or more of the following modifications: modification at the 2′- and/or 3 ′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

A 5′-UTR may be provided as a flanking region to the mRNA. A 5′-UTR may be homologous or heterologous to the coding region found in a polynucleotide. Multiple 5 ′-UTRs may be included in the flanking region and may be the same or of different sequences. Any portion of the flanking regions, including none, may be codon optimized and any may independently contain one or more different structural or chemical alterations, before and/or after codon optimization. To alter one or more properties of an mRNA, 5′-UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5 ′-UTR may have on the mRNA. Variants of the 5′-UTRs may be utilized wherein one or more nucleotides are added or removed to the termini, including A, T, C or G. 5 ′-UTRs may also be codon-optimized, or altered in any manner described herein.

mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3 ′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3 ′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3 ′-region of the polynucleotide that can prevent and/or inhibit the addition of oligio(U). In yet other cases, a mRNA, which includes the histone stem loop may be stabilized by the addition of an oligonucleotide that terminates in a 3′-deoxynucleoside, 2′,3 ′-dideoxynucleoside 3′-0-methylnucleosides, 3-0-ethylnucleosides, 3 ′-arabinosides, and other alternative nucleosides known in the art and/or described herein. In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5 ′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence. mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region. In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3 ′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3 ′-stabilizing region. The mRNA with a poly-A region may further include a 5′-cap structure. In other cases, mRNA may include a poly-A-G Quartet. The mRNA with a poly-A-G Quartet may further include a 5 ′-cap structure. In some cases, the 3′-stabilizing region which may be used to stabilize mRNA includes a poly-A region or poly-A-G Quartet. In other cases, the 3 ′-stabilizing region which may be used with the present disclosure include a chain termination nucleoside such as 3′-deoxyadenosine (cordycepin), 3 ′-deoxyuridine, 3′-deoxycytosine, 3 ′-deoxyguanosine, 3′-deoxy thymine, 2′,3′-dideoxynucleosides, such as 2′,3 ′-dideoxyadenosine, 2′,3 ′-dideoxyuridine, 2′,3 ′-dideoxycytosine, 2′, 3 ′-dideoxyguanosine, 2′,3 ′-dideoxythymine, a 2′-deoxynucleoside, or an O-methylnucleoside. In other cases, mRNA which includes a polyA region or a poly-A-G Quartet may be stabilized by an alteration to the 3 ′-region of the polynucleotide that can prevent and/or inhibit the addition of oligio(U). In yet other instances, mRNA which includes a poly-A region or a poly-A-G Quartet may be stabilized by the addition of an oligonucleotide that terminates in a 3′-deoxynucleoside, 2′,3 ′-dideoxynucleoside 3-O-methylnucleosides, 3′-O-ethylnucleosides, 3 ′-arabinosides, and other alternative nucleosides known in the art and/or described herein.

In an embodiment, the mRNA vaccines of the invention comprise lipids. The lipids and modRNA can together form nanoparticles. The lipids can encapsulate the mRNA in the form of a lipid nanoparticle (LNP) to aid cell entry and stability of the RNA/lipid nanoparticles. Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body. Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

A LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl (meth) acrylate) (PMMA), poly(ethyl(meth) acrylate), poly(butyl(meth) acrylate), poly(isobutyl(meth) acrylate), poly(hexyl(meth) acrylate), poly(isodecyl(meth) acrylate), poly(lauryl(meth) acrylate), poly(phenyl(meth) acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%.

In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density. Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/V).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo% of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo% of the LNP, LNP suspension, lyophilized LNP composition, or LNP formulation of the present disclosure is longer than the Txo% of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol. The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1,8:1,9:1, 10:1, 11:1, 12:1, 13:1, 14:1, 15:1, 16:1, 17:1, 18:1, 19:1, 20:1, 25:1,30:1,35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

In some embodiments, the ionizable lipid is a compound of Formula (IL-I):
or their N-oxides, or salts or isomers thereof, wherein:

Ri is selected from the group consisting of C5-30 alkyl, C5-20 alkenyl, —R*YR″, —YR″, and —R″M′R′; R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle; R4 is selected from the group consisting of hydrogen, a C3-6 carbocycle, —(CH2)nQ, —(CH2)nCHQR, —CHQR, —CQ(R)2, and unsubstituted C1-6 alkyl, where Q is selected from a carbocycle, heterocycle, —OR, —O(CH2)nN(R)2, —C(O)OR, —OC(O)R, —CX3, —CX2H, —CXH2, —CN, —N(R)2, —C(O)N(R)2, —N(R)C(O)R, —N(R)S(O)2R, —N(R)C(O)N(R)2, —N(R)C(S)N(R)2, —N(R)Re, N(R)S(O)2R8, —O(CH2)nOR, —N(R)C(═NR9)N(R)2, —N(R)C(═CHR9)N(R)2, —OC(O)N(R)2J-N(R)C(O)OR, —N(OR)C(O)R, —N(OR)S(O)2R, —N(OR)C(O)OR, —N(OR)C(O)N(R)2, —N(OR)C(S)N(R)2, —N(OR)C(═NR9)N(R)2, —N(OR)C(=CHR9)N(R)2, —C(=NR9)N(R)2, —C(═NR9)R, —C(O)N(R) OR, and —C(R)N(R)2C(O)OR, and each n is independently selected from 1, 2, 3, 4, and 5; each R5 is independently selected from the group consisting of C1-3 alkyl, C2-3 alkenyl, and H; each Re is independently selected from the group consisting of C1-3 alkyl, C2-3 alkenyl, and H; M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)2-, —S—S—, an aryl group, and a heteroaryl group, in which M″ is a bond, C1-13 alkyl or C2-13 alkenyl; R7 is selected from the group consisting of C1-3 alkyl, C2-3 alkenyl, and H; Re is selected from the group consisting of C3-6 carbocycle and heterocycle; R9 is selected from the group consisting of H, CN, NO₂, Ci-6 alkyl, —OR, —S(O)2R, —S(O)2N(R)2, C2-6 alkenyl, C3-6 carbocycle and heterocycle; each R is independently selected from the group consisting of C1-3 alkyl, C2-3 alkenyl, and H; each R′ is independently selected from the group consisting of Ci-is alkyl, C2-is alkenyl, —R*YR″, —YR″, and H; each R″ is independently selected from the group consisting of C3-15 alkyl and C3-15 alkenyl; each R* is independently selected from the group consisting of Ci-i2 alkyl and C2-12 alkenyl; each Y is independently a C3-6 carbocycle; each X is independently selected from the group consisting of F, Cl, Br, and I; and m is selected from 5, 6, 7, 8, 9, 10, 11, 12, and 13; and wherein when R4 is —(CH2)nQ, —(CH2)nCHQR, —CHQR, or —CQ(R)2, then (i) Q is not —N(R)2 when n is 1, 2, 3, 4 or 5, or (ii) Q is not 5, 6, or 7-membered heterocycloalkyl when n is 1 or 2.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid. As used herein, the term “PEG lipid” refers to polyethylene glycol (PEG)-modified lipids. Non-limiting examples of PEG lipids include PEG-modified phosphatidylethanolamine and phosphatidic acid, PEG-ceramide conjugates (e.g., PEG-CerCl4 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid. In some embodiments, the PEG-modified lipids are a modified form of PEG DMG. In some embodiments, the PEG-modified lipid is PEG lipid with the formula (IV):

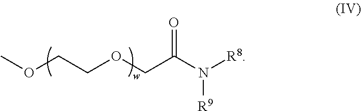

wherein R8 and R9 are each independently a straight or branched, saturated or unsaturated alkyl chain containing from 10 to 30 carbon atoms, wherein the alkyl chain is optionally interrupted by one or more ester bonds; and w has a mean value ranging from 30 to 60.

### The Vaccine BNT162b2

The BioNTech technology for the BNT162b2 (Comirnaty®; INN: tozinameran) vaccine is based on use of nucleoside-modified mRNA (modRNA) which encodes the full-length spike protein found on the surface of the SARS-COV-2 virus, triggering an immune response against infection by the virus protein (Vogel A B et al. (April 2021). Nature. 592 (7853): 283-289). See description at FIG. 2.

Table of features of sequence shown in FIG. 2

| Element | Description | Position |
|---|---|---|
| cap | A modified 5′-cap1 structure (m⁷G⁺m^{3′}-5′-ppp-5′-Am) | 1-2 |
| 5′-UTR | 5′-untranslated region derived from human alpha-globin | 3-54 |
| | RNA with an optimized Kozak sequence | |
| sig | S glycoprotein signal peptide (extended leader sequence), | 55-102 |
| | which guides translocation of the nascent polypeptide chain | |
| | into the endoplasmic reticulum. | |
| S protein_mut | Codon-optimized sequence encoding full-length SARS-CoV- | 103-3879 |
| | 2 spike (S) glycoprotein containing mutations K986P and | |
| | V987P to ensure the S glycoprotein remains in an | |
| | antigenically optimal pre-fusion conformation; stop codons: | |
| | 3874-3879 (underlined) | |
| 3′-UTR | The 3′ untranslated region comprises two sequence elements | 3880-4174 |
| | derived from the amino-terminal enhancer of split (AES) | |
| | mRNA and the mitochondrial encoded 12S ribosomal RNA to | |
| | confer RNA stability and high total protein expression. | |
| poly(A) | A 110-nucleotide poly(A)-tail consisting of a stretch of 30 | 4175-4284 |
| | adenosine residues, followed by a 10-nucleotide linker | |
| | sequence and another 70 adenosine residues. | |

The vaccine candidate BNT162b2 was chosen as the most promising among three others with similar technology developed by BioNTech (Mulligan M J, et al. (October 2020). Nature. 586 (7830): 589-593, Vogel A B et al. (April 2021). Nature. 592 (7853): 283-289). Before choosing BNT162b2, BioNTech and Pfizer had conducted Phase I trials on BNT162b1 in Germany and the United States, while Fosun performed a Phase I trial in China. In these Phase I studies, BNT162b2 was shown to have a better safety profile than the other three BioNTech candidates (Gaebler C, Nussenzweig M C (October 2020). Nature. 586 (7830): 501-2).

### Sequence of BNT162b2

The modRNA sequence of the vaccine is 4,284 nucleotides long (see FIG. 2). It consists of a five-prime cap; a five prime untranslated region derived from the sequence of human alpha globin; a signal peptide (bases 55-102) and two proline substitutions (K986P and V987P, designated “2P”) that cause the spike to adopt a prefusion-stabilized conformation reducing the membrane fusion ability, increasing expression and stimulating neutralizing antibodies (Walsh E E et al. (October 2020). The New England Journal of Medicine. 383 (25): 2439; Pallesen J, et al. (August 2017). PNAS. 114 (35): E7348-E7357); a codon-optimized gene of the full-length spike protein of SARS-COV-2 (bases 103-3879); followed by a three prime untranslated region (bases 3880-4174) combined from AES and mtRNR1 selected for increased protein expression and mRNA stability and a poly(A) tail comprising 30 adenosine residues, a 10-nucleotide linker sequence, and 70 other adenosine residues (bases 4175-4284). The sequence contains no uridine residues; they are replaced by 1-methyl-3′-pseudouridylyl. The 2P proline substitutions in the spike proteins were originally developed for a MERS vaccine by researchers at the National Institute of Allergy and Infectious Diseases' Vaccine Research Center, Scripps Research, and Jason Mclellan's team (at the University of Texas at Austin, previously at Dartmouth College).

### Composition of BNT162b2

In addition to the mRNA molecule, the vaccine contains the following inactive ingredients (excipients):
ALC-0315, ((4-hydroxybutyl) azanediyl)bis(hexane-6,1-diyl)bis(2-hexyldecanoate)
ALC-0159, 2-[(polyethylene glycol)-2000]—N,N-ditetradecylacetamide
1,2-distearoyl-sn-glycero-3-phosphocholine (DSPC)
cholesterol
dibasic sodium phosphate dihydrate
monobasic potassium phosphate
potassium chloride
sodium chloride
sucrose
water for injection

The first four of these are lipids. The lipids are intended to encapsulate the mRNA in the form of a lipid nanoparticle to aid cell entry and stability of the RNA/lipid nanoparticles. ALC-0315 is the functional cationic lipid component of the drug product. When incorporated in lipid nanoparticles, it helps regulate the endosomal release of the RNA. During drug product manufacturing, introduction of an aqueous RNA solution to an ethanolic lipid mixture containing ALC-0315 at a specific pH leads to an electrostatic interaction between the negatively charged RNA backbone and the positively charged cationic lipid. This electrostatic interaction leads to encapsulation of RNA drug substance resulting with particle formation.

The primary function of the PEGylated lipid ALC-0159 is to form a protective hydrophilic layer that sterically stabilises the lipid nanoparticle which contributes to storage stability and reduces non-specific binding to proteins.

Cholesterol is included in the formulation to support bilayer structures in the lipid nanoparticle and to provide mobility of the lipid components within the lipid nanoparticle structure.

DSPC is a phospholipid component intended to provide a stable bilayer-forming structure to balance the non-bilayer propensity of the cationic lipid. DSPC is a non-pharmacopeial excipient and an adequate specification has been provided

The lipids and modRNA together form nanoparticles.

In some embodiments, the BNT162b2 composition includes 30 mcg of the nucleoside-modified messenger RNA encoding a mutated viral spike(S) glycoprotein of SARS-COV-2. The BNT162b2 composition for each dose includes the modRNA and the following: lipids (0.43 mg (4-hydroxybutyl) azanediyl)bis(hexane-6,1-diyl)bis(2-hexyldecanoate), 0.05 mg 2 [(polyethylene glycol)-2000]-N,N-ditetradecylacetamide, 0.09 mg 1,2-distearoyl-sn-glycero-3-phosphocholine, and 0.2 mg cholesterol), 0.01 mg potassium chloride, 0.01 mg monobasic potassium phosphate, 0.36 mg sodium chloride, 0.07 mg dibasic sodium phosphate dihydrate, and 6 mg sucrose. The diluent (0.9% Sodium Chloride Injection) contributes an additional 2.16 mg sodium chloride per dose. The BNT162b2 Vaccine can have a dosing regimen that includes two doses of 0.3 mL each, 3 weeks apart. The BNT162b2 vaccine includes mRNA having the sequence as set forth in SEQ ID NO: 1 (see FIG. 2).

The vaccine is supplied in a multidose vial as “a white to off-white, sterile, preservative-free, frozen suspension for intramuscular injection”. It must be thawed to room temperature and diluted with normal saline before administration.

The Vaccine mRNA-1273

The mRNA-1273 vaccine composition includes 100 mcg of the nucleoside-modified messenger RNA encoding a mutated viral spike(S) glycoprotein of SARS-COV-2 (see FIG. 3). The vaccine composition includes the following: lipids (SM-102; 1,2-dimyristoyl-rac-glycero-3-methoxypolyethylene glycol-2000 [PEG2000-DMG]; cholesterol; and 1,2-distearoyl-sn-glycero-3-phosphocholine [DSPC]), tromethamine, tromethamine hydrochloride, acetic acid, sodium acetate, and sucrose. The mRNA-1273 vaccine may have a dosing regimen that is two doses of 0.5 mL each, one month apart. The mRNA-1273 vaccine includes mRNA having the sequence shown in FIG. 3 (SEQ ID NO: 2). In another embodiment, the mRNA vaccine includes a sequence as shown in FIG. 3 (SEQ ID NO: 2), wherein the “spike encoding region” is replaced with a SARS-COV-2 S-antigen of a variant strain.

### 2. Pneumococcal Conjugate Vaccines (PCVs) of the Invention

Pneumococcal conjugate vaccines of the present invention will typically comprise conjugated capsular saccharide antigens (also named herein conjugates or glycoconjugates), wherein the saccharides are derived from serotypes of S. pneumoniae.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein.

Preferably, the number of S. pneumoniae capsular saccharides can range from 13 serotypes (or “v”, valences) to 20 different serotypes (25v). In one embodiment there are 12 different serotypes. In one embodiment there are 13 different serotypes. In one embodiment there are 14 different serotypes. In one embodiment there are 15 different serotypes. In one embodiment there are 16 different serotypes. In an embodiment there are 17 different serotypes. In an embodiment there are 18 different serotypes. In an embodiment there are 19 different serotypes. In an embodiment there are 20 different serotypes. The capsular saccharides are conjugated to a carrier protein to form glycoconjugates as described here below.

If the protein carrier is the same for 2 or more saccharides in the composition, the saccharides could be conjugated to the same molecule of the protein carrier (carrier molecules having 2 or more different saccharides conjugated to it) [see for instance WO 2004/083251].

In a preferred embodiment though, the saccharides are each individually conjugated to different molecules of the protein carrier (each molecule of protein carrier only having one type of saccharide conjugated to it). In said embodiment, the capsular saccharides are said to be individually conjugated to the carrier protein.

For the purposes of the invention the term ‘glycoconjugate’ or ‘conjugate’ indicates a capsular saccharide linked covalently to a carrier protein. In one embodiment a capsular saccharide is linked directly to a carrier protein. In a second embodiment a bacterial saccharide is linked to a protein through a spacer/linker.

### 2.1 Capsular Saccharide of the Invention

The term “saccharide” throughout this specification may indicate polysaccharide or oligosaccharide and includes both. In frequent embodiments, the saccharide is a polysaccharide, in particular a S. pneumoniae capsular polysaccharide.

Capsular polysaccharides are prepared by standard techniques known to those of ordinary skill in the art.

Typically, capsular polysaccharides are produced by growing each S. pneumoniae serotype in a medium (e.g., in a soy-based medium), the polysaccharides are then prepared from the bacteria culture. Bacterial strains of S. pneumoniae used to make the respective polysaccharides that are used in the glycoconjugates of the invention may be obtained from established culture collections (such as for example the Streptococcal Reference Laboratory (Centers for Disease Control and Prevention, Atlanta, GA)) or clinical specimens.

The population of the organism (each S. pneumoniae serotype) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see for example WO 2006/110381, WO 2008/118752, and U.S. Patent App. Pub. Nos. 2006/0228380, 2006/0228381, 2008/0102498 and 2008/0286838).

The individual polysaccharides are typically purified through centrifugation, precipitation, ultra-filtration, and/or column chromatography (see for example WO 2006/110352 and WO 2008/118752).

Purified polysaccharides may be activated (e.g., chemically activated) to make them capable of reacting (e.g., either directly to the carrier protein of via a linker such as an eTEC spacer) and then incorporated into glycoconjugates of the invention, as further described herein. S. pneumoniae capsular polysaccharides comprise repeating oligosaccharide units which may contain up to 8 sugar residues.

In an embodiment, capsular saccharide of the invention may be one oligosaccharide unit, or a shorter than native length saccharide chain of repeating oligosaccharide units. In an embodiment, capsular saccharide of the invention is one repeating oligosaccharide unit of the relevant serotype.

In an embodiment, capsular saccharide of the invention may be oligosaccharides. Oligosaccharides have a low number of repeat units (typically 5-15 repeat units) and are typically derived synthetically or by hydrolysis of polysaccharides.

In an embodiment, all of the capsular saccharides of the present invention and in the immunogenic compositions of the present invention are polysaccharides. High molecular weight capsular polysaccharides are able to induce certain antibody immune responses due to the epitopes present on the antigenic surface. The isolation and purification of high molecular weight capsular polysaccharides is preferably contemplated for use in the conjugates, compositions and methods of the present invention.

In some embodiments, the purified polysaccharides before conjugation have a molecular weight of between 5 kDa and 4,000 kDa. In other such embodiments, the polysaccharide has a molecular weight of between 10 kDa and 4,000 kDa; between 50 kDa and 4,000 kDa; between 50 kDa and 3,000 kDa; between 50 kDa and 2,000 kDa; between 50 kDa and 1,500 kDa; between 50 kDa and 1,000 kDa; between 50 kDa and 750 kDa; between 50 kDa and 500 kDa; between 100 kDa and 4,000 kDa; between 100 kDa and 3,000 kDa; 100 kDa and 2,000 kDa; between 100 kDa and 1,500 kDa; between 100 kDa and 1,000 kDa; between 100 kDa and 750 kDa; between 100 kDa and 500 kDa; between 100 and 400 kDa; between 200 kDa and 4,000 kDa; between 200 kDa and 3,000 kDa; between 200 kDa and 2,000 kDa; between 200 kDa and 1,500 kDa; between 200 kDa and 1,000 kDa. In an embodiment, the capsular polysaccharide has a molecular weight of between or between 200 kDa and 500 kDa. In another embodiment, the capsular polysaccharide has a molecular weight of between 100 kDa to 500 kDa.

In further embodiments, the capsular polysaccharide has a molecular weight of between 5 kDa to 100 kDa; 7 kDa to 100 kDa; 10 kDa to 100 kDa; 20 kDa to 100 kDa; 30 kDa to 100 kDa; 40 kDa to 100 kDa; 50 kDa to 100 kDa; 60 kDa to 100 kDa; 70 kDa to 100 kDa; 80 kDa to 100 kDa; 90 kDa to 100 kDa; 5 kDa to 90 KDa; 5 kDa to 80 kDa; 5 kDa to 70 kDa; 5 kDa to 60 kDa; 5 kDa to 50 kDa; 5 kDa to 40 kDa; 5 kDa to 30 kDa; 5 kDa to 20 kDa or 5 kDa to 10 kDa. Any whole number integer within any of the above ranges is contemplated as an embodiment of the disclosure.

A polysaccharide can become slightly reduced in size during normal purification procedures. Additionally, polysaccharide can be subjected to sizing techniques before conjugation. Mechanical or chemical sizing maybe employed.

In a preferred embodiment the purified polysaccharides, are capsular polysaccharide from serotypes 11, 3, 4, 5, 6A, 6B, 7F, 8, 9V, 10A, 11A, 12F, 14, 15B, 18C, 19A, 19F, 22F, 23F or 33F of S. pneumoniae, wherein the capsular polysaccharide has a molecular weight falling within one of the molecular weight ranges as described here above.

As used herein, the term “molecular weight” of polysaccharide or of carrier protein-polysaccharide conjugate refers to the weight average molecular weight (Mw) which can be measured by size exclusion chromatography (SEC) combined with multiangle laser light scattering detector (MALLS).

In some embodiments, the pneumococcal saccharides from serotypes 9V, 18C, 11A, 15B, 22F and/or 33F of the invention are O-acetylated. In some embodiments, the pneumococcal saccharides from serotypes 9V, 11A, 15B, 22F and/or 33F of the invention are O-acetylated. In a preferred embodiment, the pneumococcal saccharide from serotype 18C of the invention is de-O-acetylated. For example, saccharides of serotype 18C can be de-O-acetylated by acidic treatment (see e.g. WO2006/110381, page 37 lines 1-4).

The degree of O-acetylation of the polysaccharide can be determined by any method known in the art, for example, by proton NMR (see for example Lemercinier et al. (1996) Carbohydrate Research 296:83-96, Jones et al. (2002) J. Pharmaceutical and Biomedical Analysis 30:1233-1247, WO 2005/033148 and WO 00/56357). Another commonly used method is described in Hestrin (1949) J. Biol. Chem. 180:249-261. Preferably, the presence of O-acetyl groups is determined by ion-HPLC analysis.

The purified polysaccharides described herein are chemically activated to make the saccharides capable of reacting with the carrier protein. These pneumococcal conjugates are prepared by separate processes and formulated into a single dosage formulation as described below.

### 2.2 Glycoconjugates of the Invention

The purified saccharides are chemically activated to make the saccharides capable of reacting with the carrier protein (i.e., activated saccharides), either directly or via a linker. Once activated, each capsular saccharide is separately conjugated to a carrier protein to form a glycoconjugate. In one embodiment, each capsular saccharide is conjugated to the same carrier protein. The chemical activation of the saccharides and subsequent conjugation to the carrier protein can be achieved by the activation and conjugation methods.

Capsular polysaccharides from S. pneumoniae can be prepared as disclosed above.

In a preferred embodiment, at least one of capsular polysaccharides from serotypes 1, 3, 4, 5, 6A, 6B, 7F, 8, 9V, 10A, 11A, 12F, 14, 15B, 18C, 19A, 19F, 22F, 23F and 33F of S. pneumoniae is conjugated to the carrier protein by reductive amination (such as described in U.S. Patent Appl. Pub. Nos. 2006/0228380, 2007/184072, 2007/0231340 and 2007/0184071, WO 2006/110381, WO 2008/079653, and WO 2008/143709).

In a preferred embodiment of the present invention, the glycoconjugate from S. pneumoniae serotype 15B is prepared by reductive amination.

In a preferred embodiment of the present invention, the glycoconjugate from S. pneumoniae serotype 18C is prepared by reductive amination. In a preferred embodiment of the present invention, the glycoconjugate from S. pneumoniae serotype 6A is prepared by reductive amination. In a preferred embodiment of the present invention, the glycoconjugate from S. pneumoniae serotype 19A is prepared by reductive amination. In a preferred embodiment of the present invention, the glycoconjugate from S. pneumoniae serotype 3 is prepared by reductive amination. In a preferred embodiment of the present invention, the glycoconjugates from S. pneumoniae serotypes 6A and 19A are prepared by reductive amination. In a preferred embodiment of the present invention, the glycoconjugates from S. pneumoniae serotypes 3, 6A and 19A are prepared by reductive amination. In a preferred embodiment of the present invention, the glycoconjugate from S. pneumoniae serotype 33F is prepared using the eTEC conjugation. In a preferred embodiment of the present invention, the glycoconjugate from S. pneumoniae serotype 12F is prepared using TEMPO/NCS-reductive amination.

In an embodiment of the present invention, the glycoconjugates from S. pneumoniae serotypes 4, 6B, 9V, 14, 18C, 19F and 23F are prepared by reductive amination.

In an embodiment of the present invention, the glycoconjugates from S. pneumoniae serotypes 1, 4, 6B, 9V, 14, 18C, 19F and 23F are prepared by reductive amination. In a preferred embodiment of the present invention, the glycoconjugates from S. pneumoniae serotypes 1, 4, 5, 6B, 9V, 14, 18C, 19F and 23F are prepared by reductive amination. In an embodiment of the present invention, the glycoconjugates from S. pneumoniae serotypes 1, 4, 5, 6B, 7F, 9V, 14, 18C, 19F and 23F are prepared by reductive amination. In an embodiment of the present invention, the glycoconjugates from S. pneumoniae serotypes 1, 4, 5, 6A, 6B, 7F, 9V, 14, 18C, 19F and 23F are prepared by reductive amination. In an embodiment of the present invention, the glycoconjugates from S. pneumoniae serotypes 1, 4, 5, 6A, 6B, 7F, 9V, 14, 18C, 19A, 19F and 23F are prepared by reductive amination. In a preferred embodiment of the present invention, the glycoconjugates from S. pneumoniae serotypes 1, 3, 4, 5, 6A, 6B, 7F, 9V, 14, 18C, 19A, 19F and 23F are all prepared by reductive amination. In a preferred embodiment of the present invention, the glycoconjugates from S. pneumoniae serotypes 1, 3, 4, 5, 9V, 14 and 18C are prepared by reductive amination in aqueous solvent, the glycoconjugates from S. pneumoniae serotypes 6A, 6B, 7F, 19A, 19F and 23F are prepared by reductive amination in aprotic solvent. In a preferred embodiment of the present invention, the glycoconjugates from S. pneumoniae serotypes 1, 3, 4, 5, 9V, 14 and 18C are prepared by reductive amination in aqueous solvent, the glycoconjugates from S. pneumoniae serotypes 6A, 6B, 7F, 19A, 19F and 23F are prepared by reductive amination in DMSO.

In another preferred embodiment, the glycoconjugates from S. pneumoniae serotypes 1, 3, 4, 5, 6A, 6B, 7F, 9V, 14, 18C, 19A, 19F, 22F and 23F are all prepared by reductive amination.

In another embodiment, the glycoconjugates from S. pneumoniae serotypes 1, 3, 4, 5, 6A, 6B, 7F, 9V, 14, 18C, 19A, 19F, 23F and 33F are all prepared by reductive amination.

In another embodiment, the glycoconjugates from S. pneumoniae serotypes 1, 3, 4, 5, 6A, 6B, 7F, 9V, 14, 18C, 19A, 19F, 22F, 23F and 33F are all prepared by reductive amination. In a preferred embodiment when the vaccine is a 15-valent vaccine, the glycoconjugates from S. pneumoniae serotypes 1, 3, 4, 5, 9V, 14, 22F and 33F are prepared by reductive amination in aqueous solvent and the glycoconjugates from S. pneumoniae serotypes 6A, 6B, 7F, 18C, 19A, 19F and 23F are prepared by reductive amination in aprotic solvent. In a preferred embodiment when the vaccine is a 15-valent vaccine, the glycoconjugates from S. pneumoniae serotypes 1, 3, 4, 5, 9V, 14, 22F and 33F are prepared by reductive amination in aqueous solvent and the glycoconjugates from S. pneumoniae serotypes 6A, 6B, 7F, 18C, 19A, 19F and 23F are prepared by reductive amination in DMSO.

In another embodiment, the glycoconjugates from S. pneumoniae serotypes 1, 3, 4, 5, 6A, 6B, 7F, 9V, 14, 15B, 18C, 19A, 19F, 22F and 23F are all prepared by reductive amination.

In another preferred embodiment, the glycoconjugates from S. pneumoniae serotypes 1, 3, 4, 5, 6A, 6B, 7F, 8, 9V, 10A, 11A, 12F, 14, 15B, 18C, 19A, 19F, 22F and 23F are all prepared by reductive amination.

In another embodiment, the glycoconjugates from S. pneumoniae serotypes 1, 4, 5, 6A, 6B, 7F, 9V, 12F, 14, 18C, 19A, 19F, 22F, 23F and 33F are all prepared by reductive amination.

In another preferred embodiment, the glycoconjugates from S. pneumoniae serotypes 1, 3, 4, 5, 6A, 6B, 7F, 8, 9V, 10A, 11A, 14, 15B, 18C, 19A, 19F, 22F and 23F are all prepared by reductive amination.

In another embodiment, the glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F are all prepared by reductive amination.

In a preferred embodiment of the present invention, the glycoconjugates from S. pneumoniae serotypes 1, 3, 4, 5, 6A, 6B, 7F, 8, 9V, 10A, 11A, 14, 15B, 18C, 19A, 19F, 22F and 23F are prepared by reductive amination, the glycoconjugate from S. pneumoniae serotype 33F is prepared using the eTEC conjugation and the glycoconjugate from S. pneumoniae serotype 12F is prepared using TEMPO/NCS-reductive amination.

In a preferred embodiment of the present invention, the glycoconjugates from S. pneumoniae serotypes 1, 3, 4, 5, 9V, 11A, 14 and 18C are prepared by reductive amination in aqueous solvent, the glycoconjugates from S. pneumoniae serotypes 6A, 6B, 7F, 8, 10A, 15B, 19A, 19F, 22F and 23F are prepared by reductive amination in aprotic solvent, the glycoconjugate from S. pneumoniae serotype 33F is prepared using the eTEC conjugation and the glycoconjugate from S. pneumoniae serotype 12F is prepared using TEMPO/NCS-reductive amination in aqueous solvent.

In a preferred embodiment of the present invention, the glycoconjugates from S. pneumoniae serotypes 1, 3, 4, 5, 9V, 11A, 14 and 18C are prepared by reductive amination in aqueous solvent, the glycoconjugates from S. pneumoniae serotypes 6A, 6B, 7F, 8, 10A, 15B, 19A, 19F, 22F and 23F are prepared by reductive amination in DMSO, the glycoconjugate from S. pneumoniae serotype 33F is prepared using the eTEC conjugation and the glycoconjugate from S. pneumoniae serotype 12F is prepared using TEMPO/NCS-reductive amination in aqueous solvent.

Reductive amination involves two steps, (1) oxidation of the polysaccharide, (2) reduction of the activated polysaccharide and a carrier protein to form a conjugate. Before oxidation, the polysaccharide is optionally hydrolyzed. Mechanical or chemical hydrolysis maybe employed. Chemical hydrolysis maybe conducted using acetic acid.

The oxidation step may involve reaction with periodate. For the purpose of the present invention, the term “periodate” includes both periodate and periodic acid; the term also includes both metaperiodate (IO₄⁻) and orthoperiodate (IO₆⁵⁻) and includes the various salts of periodate (e.g., sodium periodate and potassium periodate). In an embodiment the capsular polysaccharide is oxidized in the presence of metaperiodate, preferably in the presence of sodium periodate (NaIO₄). In another embodiment the capsular polysaccharide is oxydized in the presence of orthoperiodate, preferably in the presence of periodic acid.

In an embodiment, the oxidizing agent is a stable nitroxyl or nitroxide radical compound, such as piperidine-N-oxy or pyrrolidine-N-oxy compounds, in the presence of an oxidant to selectively oxidize primary hydroxyls (as described in WO 2014/097099). In said reaction, the actual oxidant is the N-oxoammonium salt, in a catalytic cycle. In an aspect, said stable nitroxyl or nitroxide radical compound are piperidine-N-oxy or pyrrolidine-N-oxy compounds. In an aspect, said stable nitroxyl or nitroxide radical compound bears a TEMPO (2,2,6,6-tetramethyl-1-piperidinyloxy) or a PROXYL (2,2,5,5-tetramethyl-1-pyrrolidinyloxy) moiety. In an aspect, said stable nitroxyl radical compound is TEMPO or a derivative thereof. In an aspect, said oxidant is a molecule bearing a N-halo moiety. In an aspect, said oxidant is selected from the group consisting of N-ChloroSuccinimide, N-Bromosuccinimide, N-lodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In a preferred embodiment, capsular polysaccharides from serotypes 12F S. pneumoniae are conjugated to the carrier protein by reductive amination, wherein the oxidizing agent is 2,2,6,6-Tetramethyl-1-piperidinyloxy (TEMPO) free radical and N-Chlorosuccinimide (NCS) as the cooxidant (as described in WO 2014/097099). Therefore in one aspect, the glycoconjugates from S. pneumoniae serotype 12F are obtainable by a method comprising the steps of: a) reacting a 12F saccharide with 2,2,6,6-tetramethyl-1-piperidinyloxy (TEMPO) and N-chlorosuccinimide (NCS) in an aqueous solvent to produce an activated saccharide; and b) reacting the activated saccharide with a carrier protein comprising one or more amine groups (said method is designated “TEMPO/NCS-reductive amination”). 20)

Optionally the oxidation reaction is quenched by addition of a quenching agent. The quenching agent maybe selected from vicinal diols, 1,2-aminoalcohols, amino acids, glutathione, sulfite, bisulfate, dithionite, metabisulfite, thiosulfate, phosphites, hypophosphites or phosphorous acid (such as glycerol, ethylene glycol, propan-1,2-diol, butan-1,2-diol or butan-2,3-diol, ascorbic acid). Following the oxidation step of the polysaccharide, the polysaccharide is said to be activated and is referred to an “activated polysaccharide” here below. The activated polysaccharide and the carrier protein may be lyophilised (freeze-dried), either independently (discrete lyophilization) or together (co-lyophilized). In one embodiment the activated polysaccharide and the carrier protein are co-lyophilized. In another embodiment the activated polysaccharide and the carrier protein are lyophilized independently. In one embodiment the lyophilization takes place in the presence of a non-reducing sugar, possible non-reducing sugars include sucrose, trehalose, raffinose, stachyose, melezitose, dextran, mannitol, lactitol and palatinit.

The second step of the conjugation process is the reduction of the activated polysaccharide and a carrier protein to form a conjugate (so-called reductive amination), using a reducing agent. Reducing agents which are suitable include the cyanoborohydrides (such as sodium cyanoborohydride, sodium triacetoxyborohydride or sodium or zinc borohydride in the presence of Bronsted or Lewis acids), amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB) or borohydride exchange resin. In one embodiment the reducing agent is sodium cyanoborohydride.

In an embodiment, the reduction reaction is carried out in aqueous solvent (e.g., selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, 7.0 and 8.0, or 7.0 and 7.5), in another embodiment the reaction is carried out in aprotic solvent. In an embodiment, the reduction reaction is carried out in DMSO (dimethylsulfoxide) or in DMF (dimethylformamide) solvent. The DMSO or DMF solvent may be used to reconstitute the activated polysaccharide and carrier protein which has been lyophilized.

At the end of the reduction reaction, there may be unreacted aldehyde groups remaining in the conjugates, these may be capped using a suitable capping agent. In one embodiment this capping agent is sodium borohydride (NaBH₄). Following the conjugation (the reduction reaction and optionally the capping), the glycoconjugates may be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. In an embodiment, the glycoconjugates are purified by diafilitration or ion exchange chromatography or size exclusion chromatography.

In one embodiment the glycoconjugates are sterile filtered.

In an embodiment, the glycoconjugates of the invention are prepared using the eTEC conjugation, such as described in WO 2014/027302. Said glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC linked glycoconjugates of the invention may be represented by the general formula (I):

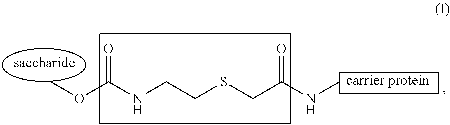

where the atoms that comprise the eTEC spacer are contained in the central box.

The eTEC spacer includes seven linear atoms (i.e., —C(O)NH(CH₂)₂SCH₂C(O)—) and provides stable thioether and amide bonds between the saccharide and carrier protein. Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond. In said glycoconjugates of the invention, the saccharide may be a polysaccharide or an oligosaccharide. The carrier protein may be selected from any suitable carrier as described herein or known to those of skill in the art. In frequent embodiments, the saccharide is a polysaccharide. In some such embodiments, the carrier protein is CRM₁₉₇. In some such embodiments, the eTEC linked glycoconjugate comprises a S. pneumoniae serotype 33F capsular polysaccharide.

In particularly preferred embodiments, the eTEC linked glycoconjugate comprises a pneumococcal serotype 33F (Pn33F) capsular polysaccharide, which is covalently conjugated to CRM₁₉₇ through an eTEC spacer (serotype 33F eTEC linked glycoconjugates).

In some embodiments, the glycoconjugate from S. pneumoniae serotypes 1, 7F, 9V and/or 18C of the invention are O-acetylated. In some embodiments, the glycoconjugate from S. pneumoniae serotypes 1, 7F and 9V is O-acetylated and the glycoconjugate from S. pneumoniae serotype 18C is de-O-acetylated.

In some embodiments, the glycoconjugates of the present invention comprise a saccharide having a molecular weight of between 5 kDa and 2,000 kDa. In other such embodiments, the saccharide has a molecular weight of between 50 kDa and 1,000 kDa. In other such embodiments, the saccharide has a molecular weight of between 70 kDa and 900 kDa. In other such embodiments, the saccharide has a molecular weight of between 100 kDa and 800 kDa. In other such embodiments, the saccharide has a molecular weight of between 200 kDa and 600 kDa. In other such embodiments, the saccharide has a molecular weight of between 100 kDa and 500 kDa. In other such embodiments, the saccharide has a molecular weight of between 100 kDa and 400 kDa. In other such embodiments, the saccharide has a molecular weight of between 150 kDa and 300 kDa. In further embodiments, the saccharide has a molecular weight of between 5 kDa to 100 kDa; 10 kDa to 100 kDa; 20 kDa to 100 kDa; 30 kDa to 100 kDa; 40 kDa to 100 kDa; 50 kDa to 100 kDa; 60 kDa to 100 kDa; 70 kDa to 100 kDa; 80 kDa to 100 kDa; 90 kDa to 100 kDa; 5 kDa to 90 KDa; 5 kDa to 80 kDa; 5 kDa to 70 kDa; 5 kDa to 60 kDa; 5 kDa to 50 kDa; 5 kDa to 40 kDa; 5 kDa to 30 kDa; 5 kDa to 20 kDa or 5 kDa to 10 kDa. Any whole number integer within any of the above ranges is contemplated as an embodiment of the disclosure. In some such embodiments, the glycoconjugate is prepared using reductive amination.

In some embodiments, the glycoconjugate of the invention has a molecular weight of between 100 kDa and 15,000 kDa. In some embodiments, the glycoconjugate of the invention has a molecular weight of between 500 kDa and 10,000 kDa. In some embodiments, the glycoconjugate of the invention has a molecular weight of between 2,000 kDa and 10,000 kDa. In some embodiments, the glycoconjugate of the invention has a molecular weight of between 3,000 kDa and 8,000 kDa kDa. In some embodiments, the glycoconjugate of the invention has a molecular weight of between 3,000 kDa and 5,000 kDa. In other embodiments, the glycoconjugate has a molecular weight of between 500 kDa and 10,000 kDa. In other embodiments, glycoconjugate has a molecular weight of between 1,000 kDa and 8,000 kDa. In still other embodiments, the glycoconjugate has a molecular weight of between 2,000 kDa and 8,000 kDa or between 3,000 kDa and 7,000 kDa.

The molecular weight of the glycoconjugate is measured by SEC-MALLS. Any whole number integer within any of the above ranges is contemplated as an embodiment of the disclosure. Another way to characterize the glycoconjugates of the invention is by the number of lysine residues in the carrier protein (e.g., CRM₁₉₇) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered, compared to the carrier protein starting material used to generate the conjugate materials. In a preferred embodiment, the degree of conjugation of the glycoconjugates of the invention is between 2 and 15. In an embodiment, the degree of conjugation of the glycoconjugates of the invention is between 2 and 13. In an embodiment, the degree of conjugation of the glycoconjugates of the invention is between 2 and 10. In an embodiment, the degree of conjugation of the glycoconjugates of the invention is between 2 and 8. In an embodiment, the degree of conjugation of the glycoconjugates of the invention is between 2 and 6. In an embodiment, the degree of conjugation of the glycoconjugates of the invention is between 3 and 10. In an embodiment, the degree of conjugation of the glycoconjugates of the invention is between 3 and 6. In an embodiment, the degree of conjugation of the glycoconjugates of the invention is between 5 and 10. In an embodiment, the degree of conjugation of the glycoconjugates of the invention is between 8 and 12. In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5. In an embodiment, the degree of conjugation of the glycoconjugate of the invention is about 6. In an embodiment, the degree of conjugation of the glycoconjugate of the invention is about 8. In an embodiment, the degree of conjugation of the glycoconjugate of the invention is about 10. In an embodiment, the degree of conjugation of the glycoconjugate of the invention is about 12. In an embodiment, the degree of conjugation of the glycoconjugate of the invention is about 15. In a preferred embodiment, the degree of conjugation of the glycoconjugate of the invention is between 4 and 7. In some such embodiments, the carrier protein is CRM₁₉₇. The glycoconjugates of the invention may also be characterized by the ratio (weight/weight) of saccharide to carrier protein. In some embodiments, the ratio of polysaccharide to carrier protein in the glycoconjugate (w/w) is between 0.5 and 3. In some embodiments, the ratio of polysaccharide to carrier protein in the glycoconjugate (w/w) is about 0.8. In some embodiments, the ratio of polysaccharide to carrier protein in the glycoconjugate (w/w) is about 0.9. In some embodiments, the ratio of polysaccharide to carrier protein in the glycoconjugate (w/w) is about 1.0. In some embodiments, the ratio of polysaccharide to carrier protein in the glycoconjugate (w/w) is about 1.2. In some embodiments, the ratio of polysaccharide to carrier protein in the glycoconjugate (w/w) is about 1.5. In some embodiments, the ratio of polysaccharide to carrier protein in the glycoconjugate (w/w) is about 1.8. In some embodiments, the ratio of polysaccharide to carrier protein in the glycoconjugate (w/w) is about 2.0. In some embodiments, the ratio of polysaccharide to carrier protein in the glycoconjugate (w/w) is about 2.5. In some embodiments, the ratio of polysaccharide to carrier protein in the glycoconjugate (w/w) is about 3.0. In other embodiments, the saccharide to carrier protein ratio (w/w) is between 0.5 and 2.0. In other embodiments, the saccharide to carrier protein ratio (w/w) is between 0.5 and 1.5. In further embodiments, the saccharide to carrier protein ratio (w/w) is between 0.8 and 1.2. In a preferred embodiment, the ratio of capsular polysaccharide to carrier protein in the conjugate is between 0.9 and 1.1. In some such embodiments, the carrier protein is CRM₁₉₇.

The glycoconjugates and immunogenic compositions of the invention may contain free saccharide that is not covalently conjugated to the carrier protein, but is nevertheless present in the glycoconjugate composition. The free saccharide may be non-covalently associated with (i.e., non-covalently bound to, adsorbed to, or entrapped in or with) the glycoconjugate. In a preferred embodiment, the glycoconjugate comprises less than about 50%, 45%, 40%, 35%, 30%, 25%, 20% or 15% of free polysaccharide compared to the total amount of polysaccharide. In a preferred embodiment the glycoconjugate comprises less than about 25% of free polysaccharide compared to the total amount of polysaccharide. In a preferred embodiment the glycoconjugate comprises less than about 20% of free polysaccharide compared to the total amount of polysaccharide. In a preferred embodiment the glycoconjugate comprises less than about 15% of free polysaccharide compared to the total amount of polysaccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression,

$${K}_{d} = ( {V}_{e} - {V}_{0} ) / ( {V}_{i} - {V}_{0} ) .$$

In a preferred embodiment, at least 30% of the glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 45%, 50%, 55%, 60%, 65%, 70%, 75%, 80%, or 85% of the glycoconjugate has a K_{d} below or equal to 0.3 in a CL-4B column. In a preferred embodiment, at least 60% of the glycoconjugate has a K_{d} below or equal to 0.3 in a CL-4B column. In a preferred embodiment, between 50% and 80% of the glycoconjugate has a K_{d} below or equal to 0.3 in a CL-4B column. In a preferred embodiment, between 65% and 80% of the glycoconjugate has a K_{d} below or equal to 0.3 in a CL-4B column. The frequency of attachment of the saccharide chain to a lysine on the carrier protein is another parameter for characterizing the glycoconjugates of the invention. For example, in some embodiments, at least one covalent linkage between the carrier protein and the polysaccharide occurs for every 4 saccharide repeat units of the polysaccharide. In another embodiment, the covalent linkage between the carrier protein and the polysaccharide occurs at least once in every 10 saccharide repeat units of the polysaccharide. In another embodiment, the covalent linkage between the carrier protein and the polysaccharide occurs at least once in every 15 saccharide repeat units of the polysaccharide. In a further embodiment, the covalent linkage between the carrier protein and the polysaccharide occurs at least once in every 25 saccharide repeat units of the polysaccharide.

In frequent embodiments, the carrier protein is CRM₁₉₇ and the covalent linkage via an eTEC spacer between the CRM₁₉₇ and the polysaccharide occurs at least once in every 4, 10, 15 or 25 saccharide repeat units of the polysaccharide.

In other embodiments, the conjugate comprises at least one covalent linkage between the carrier protein and saccharide for every 5 to 10 saccharide repeat units. In other embodiments, the conjugate comprises at least one covalent linkage between the carrier protein and saccharide every 2 to 7 saccharide repeat units. In other embodiments, the conjugate comprises at least one covalent linkage between the carrier protein and saccharide for every 7 to 12 saccharide repeat units. In other embodiments, the conjugate comprises at least one covalent linkage between the carrier protein and saccharide for every 10 to 15 saccharide repeat units. In other embodiments, the conjugate comprises at least one covalent linkage between the carrier protein and saccharide for every 4 to 8 saccharide repeat units. In other embodiments, the conjugate comprises at least one covalent linkage between the carrier protein and saccharide for every 10 to 20 saccharide repeat units In other embodiments, the conjugate comprises at least one covalent linkage between the carrier protein and saccharide for every 2 to 25 saccharide repeat units. In frequent embodiments, the carrier protein is CRM₁₉₇.

In another embodiment, at least one linkage between carrier protein and saccharide occurs for every 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In an embodiment, the carrier protein is CRM₁₉₇. Any whole number integer within any of the above ranges is contemplated as an embodiment of the disclosure.

### 2.3 Carrier Protein of the Invention

A component of the conjugate of the invention is a carrier protein to which the pneumococcal saccharide is conjugated. The terms “protein carrier” or “carrier protein” or “carrier” may be used interchangeably herein. Carrier proteins should be amenable to standard conjugation procedures.

In a preferred embodiment, the carrier protein of the conjugates is selected in the group consisiting of: DT (Diphtheria toxin), TT (tetanus toxid) or fragment C of TT, CRM₁₉₇ (a nontoxic but antigenically identical variant of diphtheria toxin), other DT mutants (such as CRM176, CRM228, CRM45 (Uchida et al. (1973) J. Biol. Chem. 218:3838-3844), CRM9, CRM102, CRM103 or CRM107; and other mutations described by Nicholls and Youle in Genetically Engineered Toxins, Ed: Frankel, Maecel Dekker Inc. (1992); deletion or mutation of Glu-148 to Asp, GIn or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169 (11): 4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa.

In a preferred embodiment, the carrier protein of the conjugates is independently selected from the group consisting of TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile and PsaA. In an embodiment, the carrier protein of the conjugates of the invention is DT (Diphtheria toxoid). In another embodiment, the carrier protein of the conjugates of the invention is TT (tetanus toxid). In another embodiment, the carrier protein of the conjugates of the invention is PD (H. influenzae protein D; see, e.g., EP0594610 B).

In a preferred embodiment, the pneumococcla capsular saccharides of the invention are conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382. In a preferred embodiment, all the pneumococcal capsular saccharides of the invention are individually conjugated to CRM₁₉₇ protein.

In an embodiment, the pneumococcal capsular saccharides of the invention are conjugated to CRM₁₉₇ protein or the A chain of CRM₁₉₇ (see CN103495161). In an embodiment, the pneumococcal capsular saccharides of the invention are conjugated the A chain of CRM₁₉₇ obtained via expression by genetically recombinant E. coli (see CN103495161). In an embodiment, the capsular saccharides of the invention are all conjugated to CRM₁₉₇. In an embodiment, the capsular saccharides of the invention are all conjugated to the A chain of CRM₁₉₇.

Accordingly, in frequent embodiments, the glycoconjugates of the invention comprise CRM₁₉₇ as the carrier protein, wherein the pneumococcal capsular polysaccharide is covalently linked to CRM₁₉₇.

### 2.4 Pneumococcal Conjugate Vaccines (PCV) of the Invention

In an embodiment, the number of different S. pneumoniae capsular saccharide serotypes of the pneumococcal conjugate vaccines can range from 13 serotypes (or “v”, valence) to 20 different serotypes (from 13v to 20v). In one embodiment the pneumococcal conjugate vaccine of the invention is a 13-valent pneumococcal vaccine. In one embodiment the pneumococcal conjugate vaccine of the invention is a 14-valent pneumococcal vaccine. In one embodiment the pneumococcal conjugate vaccine of the invention is a 15-valent pneumococcal vaccine. In one embodiment the pneumococcal conjugate vaccine of the invention is a 16-valent pneumococcal vaccine. In one embodiment the pneumococcal conjugate vaccine of the invention is a 17-valent pneumococcal vaccine. In one embodiment the pneumococcal conjugate vaccine of the invention is a 18-valent pneumococcal vaccine. In one embodiment the pneumococcal conjugate vaccine of the invention is a 19-valent pneumococcal vaccine. In one embodiment the pneumococcal conjugate vaccine of the invention is a 20-valent pneumococcal vaccine.

The capsular saccharides are conjugated to a carrier protein to form glycoconjugates as described here above.

Preferably, all the glycoconjugates of the above pneumococcal conjugate vaccines are individually conjugated to the carrier protein.

In an embodiment, the glycoconjugates from S. pneumoniae are all individually conjugated to CRM₁₉₇.

In one embodiment the pneumococcal conjugate vaccine of the invention comprises 13 glycoconjugates from a Streptococcus pneumoniae serotype selected from the group consisting of serotypes 1, 3, 4, 5, 6A, 6B, 7F, 8, 9V, 10A, 11A, 12F, 14, 15B, 18C, 19A, 19F, 23F, 22F and 33F. Preferably, said glycoconjugates are all individually conjugated to CRM₁₉₇.

In an embodiment, the pneumococcal conjugate vaccine of the invention is a 13-valent pneumococcal conjugate vaccine wherein said 13 conjugates consists of glycoconjugates from S. pneumoniae serotypes 1, 3, 4, 5, 6A, 6B, 7F, 9V, 14, 18C, 19A, 19F and 23F. Preferably, said glycoconjugates are all individually conjugated to CRM₁₉₇.

In an embodiment, the pneumococcal conjugate vaccine of the invention is a 14-valent pneumococcal conjugate vaccine wherein said 14 conjugates consists of glycoconjugates from S. pneumoniae serotypes 1, 3, 4, 5, 6A, 6B, 7F, 9V, 14, 18C, 19A, 19F, 23F. Preferably, said glycoconjugates are all individually conjugated to CRM₁₉₇.

In an embodiment, the pneumococcal conjugate vaccine of the invention is a 14-valent pneumococcal conjugate vaccine wherein said 14 conjugates consists of glycoconjugates from S. pneumoniae serotypes 1, 3, 4, 5, 6A, 6B, 7F, 9V, 14, 18C, 19A, 19F, 23F and 33F. Preferably, said glycoconjugates are all individually conjugated to CRM₁₉₇.

In an embodiment, the pneumococcal conjugate vaccine of the invention is a 15-valent pneumococcal conjugate vaccine wherein said 15 conjugates consists of glycoconjugates from S. pneumoniae serotypes 1, 3, 4, 5, 6A, 6B, 7F, 9V, 14, 18C, 19A, 19F, 23F, 22F and 33F. Preferably, said glycoconjugates are all individually conjugated to CRM₁₉₇.

In an embodiment, the pneumococcal conjugate vaccine of the invention is a 16-valent pneumococcal conjugate vaccine wherein said 16 conjugates consists of glycoconjugates from S. pneumoniae serotypes 1, 3, 4, 5, 6A, 6B, 7F, 9V, 14, 15B, 18C, 19A, 19F, 23F, 22F and 33F. Preferably, said glycoconjugates are all individually conjugated to CRM₁₉₇.

In an embodiment, the pneumococcal conjugate vaccine of the invention is a 17-valent pneumococcal conjugate vaccine wherein said 17 conjugates consists of glycoconjugates from S. pneumoniae serotypes 1, 3, 4, 5, 6A, 6B, 7F, 9V, 11A, 14, 15B, 18C, 19A, 19F, 23F, 22F and 33F. Preferably, said glycoconjugates are all individually conjugated to CRM₁₉₇.

In an embodiment, the pneumococcal conjugate vaccine of the invention is a 18-valent pneumococcal conjugate vaccine wherein said 18 conjugates consists of glycoconjugates from S. pneumoniae serotypes 1, 3, 4, 5, 6A, 6B, 7F, 9V, 11A, 12F, 14, 15B, 18C, 19A, 19F, 23F, 22F and 33F. Preferably, said glycoconjugates are all individually conjugated to CRM₁₉₇.

In an embodiment, the pneumococcal conjugate vaccine of the invention is a 19-valent pneumococcal conjugate vaccine wherein said 19 conjugates consists of glycoconjugates from S. pneumoniae serotypes 1, 3, 4, 5, 6A, 6B, 7F, 8, 9V, 11A, 12F, 14, 15B, 18C, 19A, 19F, 23F, 22F and 33F. Preferably, said glycoconjugates are all individually conjugated to CRM₁₉₇.

In a preferred embodiment, the pneumococcal conjugate vaccine of the invention is a 20-valent pneumococcal conjugate vaccine wherein said 20 conjugates consists of glycoconjugates from S. pneumoniae serotypes 1, 3, 4, 5, 6A, 6B, 7F, 8, 9V, 10A, 11A, 12F, 14, 15B, 18C, 19A, 19F, 23F, 22F and 33F. Preferably, said glycoconjugates are all individually conjugated to CRM₁₉₇.

In an embodiment, the pneumococcal conjugate vaccine of the invention is PREVNAR 13® (PREVENAR 13® in some countries). PREVNAR 13® is a 13-valent PCV where the 13 conjugates consist of glycoconjugates from S. pneumoniae serotypes 1, 3, 4, 5, 6A, 6B, 7F, 9V, 14, 18C, 19A, 19F and 23F all individually conjugated to CRM₁₉₇. The glycoconjugates are prepared by reductive amination.

In an embodiment, the pneumococcal conjugate vaccine of the invention is V114 developped by Merck. V114 is a 15-valent PCV where the 15 conjugates consist of glycoconjugates from S. pneumoniae serotypes 1, 3, 4, 5, 6A, 6B, 7F, 9V, 14, 18C, 19A, 19F, 22F, 23F and 33F all individually conjugated to CRM₁₉₇. The glycoconjugates from S. pneumoniae serotypes 1, 3, 4, 5, 9V, 14, 22F and 33F are prepared by reductive amination in aqueous solvent and the glycoconjugates from S. pneumoniae serotypes 6A, 6B, 7F, 18C, 19A, 19F and 23F are prepared by reductive amination in DMSO.

In an embodiment, the pneumococcal conjugate vaccine of the invention is 20vPnC. 20vPnC is a 20-valent PCV where the 20 conjugates consist of glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F all individually conjugated to CRM₁₉₇.

### 3. Dosage of the Pneumococcal Conjugate Vaccine

### 3.1 Polysaccharide Amount

The amount of glycoconjugate(s) in each dose is selected as an amount which induces an immunoprotective response without significant, adverse side effects in typical vaccinees. Such amount will vary depending upon which specific immunogen is employed and how it is presented.

The amount of a particular glycoconjugate in an immunogenic composition can be calculated based on total polysaccharide for that conjugate (conjugated and non-conjugated). For example, a glycoconjugate with 20% free polysaccharide will have about 80 μg of conjugated polysaccharide and about 20 μg of non-conjugated polysaccharide in a 100 μg polysaccharide dose. The amount of glycoconjugate can vary depending upon the streptococcal serotype. The saccharide concentration can be determined by the uronic acid assay.

The “immunogenic amount” of the different polysaccharide components in the immunogenic composition, may diverge and each may comprise about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg, about 30.0 μg, about 40.0 μg, about 50.0 μg, about 60.0 μg, about 70.0 μg, about 80.0 μg, about 90.0 μg, or about 100.0 μg of any particular polysaccharide antigen. Generally, each dose will comprise 0.1 μg to 100 μg of polysaccharide for a given serotype, particularly 0.5 μg to 20 μg, more particularly 1 μg to 10 μg, and even more particularly 2 μg to 5 μg. Any whole number integer within any of the above ranges is contemplated as an embodiment of the disclosure.

In an embodiment, each dose will comprise 1 μg, 2 μg, 3 μg, 4 μg, 5 μg, 6 μg, 7 μg, 8 μg, 9 μg, 10 μg, 15 μg or 20 μg of polysaccharide for a given serotype.

### 3.2 Carrier Amount

Generally, each dose will comprise 5 μg to 150 μg of carrier protein, particularly 10 μg to 100 μg of carrier protein, more particularly 15 μg to 100 μg of carrier protein, more particularly 25 to 75 μg of carrier protein, more particularly 30 μg to 70 μg of carrier protein, more particularly 30 to 60 μg of carrier protein, more particularly 30 μg to 50 μg of carrier protein and even more particularly 40 to 60 μg of carrier protein. In an embodiment, said carrier protein is CRM₁₉₇.

In an embodiment, each dose will comprise about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, 68 μg, about 69 μg, about 70 μg, about 71 μg, about 72 μg, about 73 μg, about 74 μg or about 75 μg of carrier protein. In an embodiment, said carrier protein is CRM₁₉₇.

### 4. Adjuvant(s) of the Pneumococcal Conjugate Vaccine

In some embodiments, the pneumococcal conjugate vaccines disclosed herein may further comprise at least one, two or three adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (TWEEN® 80), 0.5% w/v sorbitan trioleate (Span 85)), water-in-oil emulsions such as MONTANIDE™, and poly(D,L-lactide-co-glycolide) (PLG) microparticles or nanoparticles.

In an embodiment, the pneumococcal conjugate vaccines disclosed herein comprise aluminum salts (alum) as adjuvant (e.g., aluminum phosphate, aluminum sulfate or aluminum hydroxide). In a preferred embodiment, the pneumococcal conjugate vaccines disclosed herein comprise aluminum phosphate or aluminum hydroxide as adjuvant. In an embodiment, the pneumococcal conjugate vaccines disclosed herein comprise from 0.1 mg/mL to 1 mg/ml or from 0.2 mg/mL to 0.3 mg/mL of elemental aluminum in the form of aluminum phosphate. In an embodiment, the pneumococcal conjugate vaccines disclosed herein comprise about 0.25 mg/ml of elemental aluminum in the form of aluminum phosphate.

In an embodiment the pneumococcal conjugate vaccines of the invention comprises aluminum salt (alum) (e.g., aluminum phosphate, aluminum sulfate or aluminum hydroxide). In an embodiment, the pneumococcal conjugate vaccines of the invention comprise aluminum phosphate or aluminum hydroxide as adjuvant.

### 5. Formulation of the Pneumococcal Conjugate Vaccine

The pneumococcal conjugate vaccines of the invention may be formulated in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions of the invention.

In an embodiment, the pneumococcal conjugate vaccines of the invention is in liquid form, preferably in aqueous liquid form.

In an embodiment the pneumococcal conjugate vaccines of the invention comprises a buffer. In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM.

In an embodiment, the pneumococcal conjugate vaccines of the invention comprises a salt. In some embodiments, the salt is selected from the groups consisting of magnesium chloride, potassium chloride, sodium chloride and a combination thereof. In one particular embodiment, the salt is sodium chloride. In one particular embodiment, the pneumococcal conjugate vaccine of the invention comprises sodium chloride at 150 mM.

In an embodiment, the pneumococcal conjugate vaccines of the invention comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-1 01, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w). In certain embodiments, the pneumococcal conjugate vaccine of the invention has a pH of 5.5 to 7.5, more preferably a pH of 5.6 to 7.0, even more preferably a pH of 5.8 to 6.0.

A typical dose of the pneumococcal conjugate vaccines of the invention for injection has a volume of 0.1 mL to 2 mL, more preferably 0.2 mL to 1 mL, even more preferably a volume of about 0.5 mL.

### 6. Method for Eliciting an Immunoprotective Response

In an embodiment the invention relates to a method for eliciting an immunoprotective response in a human against S. pneumoniae and Severe Acute Respiratory Syndrome Coronavirus 2 (SARS-COV-2), the method comprising co-administering (e.g. concomitantly or concurrently) to the human an effective dose of a pneumococcal conjugate vaccine (PCV) and an mRNA vaccine against SARS-COV-2. In an embodiment said pneumococcal conjugate vaccine and said mRNA vaccine against SARS-COV-2 are any of the vaccine disclosed herein.

In a preferred embodiment an immunoprotective response against S. pneumoniae can be measured by any method known in the art, such as IgG level, fold rise in IgG level from before to after vaccination, OPA titers and/or fold rise in OPA titers from before to after vaccination (e.g. at least a 4-fold rise in OPA titers).

For each serotype of S. pneumoniae the level of IgG antibodies which are capable of binding S. pneumoniae polysaccharide can be determined by ELISA assay.

In the ELISA (Enzyme-linked Immunosorbent Assay) method, antibodies from the sera of vaccinated subjects are incubated with polysaccharides which have been adsorbed to a solid support. The bound antibodies are detected using enzyme-conjugated secondary detection antibodies. In an embodiment said ELISA assay is the standardized ELISA assay as defined by the WHO in the “Training Manual For Enzyme Linked Immunosorbent Assay For The Quantitation Of Streptococcus Pneumoniae Serotype Specific IgG (Pn PS ELISA). (007sp Version)” (available for example at https://www.vaccine.uab.edu/uploads/mdocs/ELISAProtocol(007sp).pdf, accessed on May 3, 2021).

The ELISA measures type specific IgG anti-S. pneumoniae capsular polysaccharide (PS) antibodies present in human serum. When dilutions of human sera are added to type-specific capsular PS-coated microtiter plates, antibodies specific for that capsular PS bind to the microtiter plates. The antibodies bound to the plates are detected using a goat anti-human IgG alkaline phosphatase-labeled antibody followed by a p-nitrophenyl phosphate substrate. The optical density of the colored end product is proportional to the amount of anticapsular PS antibody present in the serum.

In an embodiment an immunoprotective response against S. pneumoniae can be measured by IgG level as determined by ELISA assay (such as the standardized ELISA assay as defined by the WHO), where the subject achieves a pre-specified level of pneumococcal IgG concentrations after vaccination for a given serotype. In an embodiment, said level is measured about 1 month after vaccination. In an embodiment the pre-specified levels of IgG concentrations after vaccination are as follows: for serotype 1, 3, 4, 6A, 7F, 9V, 14, 18C, 19F, 23F, 8, 10A, 11A, 12F, 15B, 22F, 33F: at least 0.35 microgram per milliliter, for serotype 5: at least 0.23 microgram per milliliter, for serotype 6B: at least 0.10 microgram per milliliter and for serotype 19A: at least 0.12 microgram per milliliter.

In a preferred embodiment an immunoprotective response against S. pneumoniae can be measured by pneumococcal OPA titers or fold rise in OPA titers from before to after vaccination (e.g. 1 month after vaccination). In such an embodiment, an immunoprotective response against S. pneumoniae can be measured by a at least 4-fold rise in OPA titers from before to after vaccination (e.g. 1 month after vaccination).

The pneumococcal opsonophagocytic assay (OPA), which measures killing of S. pneumoniae cells by phagocytic effector cells in the presence of functional antibody and complement, is considered to be an important surrogate for evaluating the effectiveness of pneumococcal vaccines.

In vitro opsonophagocytic assay (OPA) can be conducted by incubating together a mixture of Streptococcus pneumoniae cells, a heat inactivated human serum to be tested, differentiated HL-60 cells (phagocytes) and an exogenous complement source (e.g., baby rabbit complement). Opsonophagocytosis proceeds during incubation and bacterial cells that are coated with antibody and complement are killed upon opsonophagocytosis. Colony forming units (cfu) of surviving bacteria that escape from opsonophagocytosis are determined by plating the assay mixture. The OPA titer is defined as the reciprocal dilution that results in a 50% reduction in bacterial count over control wells without test serum. The OPA titer is interpolated from the two dilutions that encompass this 50% killing cut-off.

An endpoint titer of 1:8 or greater is considered a positive result in these killing type OPA. Therefore, in an embodiment an immunoprotective response against a S. pneumoniae serotype can be measured by pneumococcal OPA titer where a result is considered positive when an endpoint titer of 1:8 or greater is measured.

In some embodiment, the human subjects may have serotype specific OPA titers prior to pneumococcal vaccination due for example to natural exposures to S. pneumoniae (e.g., in case of adult subjects). Therefore, comparaison of OPA activity of pre- and post-immunization serum with the pneumococcal conjugate vaccine of the invention can be conducted by comparing the potential increase in OPA titers.

In an embodiment an immunoprotective response against a S. pneumoniae serotype can be measured by fold rise in OPA titers from before to after vaccination (e.g. 1 month after vaccination) where a at least 4-fold rise in OPA titers from before to after vaccination is considered positive.

In a preferred embodiment an immunoprotective response against SARS-COV-2 can be measured by any method known in the art, such as vaccine-induced antibody response concentrations of S-binding IgG and/or SARS-COV-2-neutralizing titres.

In a preferred embodiment an immunoprotective response against SARS-COV-2 can be measured by full-length S-binding IgG levels (antigen-specific antibodies) and/or by the neutralizing antibody titer produced. In a preferred embodiment an immunoprotective response against SARS-COV-2 can be measured by full-length S-binding IgG levels. In another preferred embodiment an immunoprotective response against SARS-COV-2 can be measured by full by the neutralizing antibody titer produced.

In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques. For example, in a clinical trial conducted in 2020, described in N Engl J Med. 2020 Dec. 31; 383 (27): 2603-2615, the 50% neutralizing geometric mean titers elicited by 30 μg of BNT162b2 in older and younger adults exceeded the geometric mean titer measured in a human convalescent serum panel.

In exemplary embodiments, the antibody titer (i.e., the amount of antigen-specific antibody (S-binding) produces in a subject) is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody liter measurement is expressed as a ratio, such as 1:40. 1:100, etc. In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000.

In an embodiment of the invention, an efficacious vaccine produces an antibody titer of greater than 1:40. In an embodiment of the invention, an efficacious vaccine produces an antibody titer of greater that 1:100. In an embodiment of the invention, an efficacious vaccine produces an antibody titer of greater than 1:400. In an embodiment of the invention, an efficacious vaccine produces an antibody titer of greater than 1:1000. In an embodiment of the invention, an efficacious vaccine produces an antibody titer of greater than 1:2000. In an embodiment of the invention, an efficacious vaccine produces an antibody titer of greater than 1:3000. In an embodiment of the invention, an efficacious vaccine produces an antibody titer of greater than 1:4000. In an embodiment of the invention, an efficacious vaccine produces an antibody titer of greater than 1:500. In an embodiment of the invention, an efficacious vaccine produces an antibody titer of greater than 1:6000. In an embodiment of the invention, an efficacious vaccine produces an antibody titer of greater than 1:7500. In an embodiment of the invention, an efficacious vaccine produces an antibody titer of greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In an embodiment of the invention, the antibody titer is produced or reached by 10 days following vaccination. In an embodiment of the invention, the antibody titer is produced or reached by 20 days following vaccination. In an embodiment of the invention, the antibody titer is produced or reached by 30 days following vaccination. In an embodiment of the invention, the antibody titer is produced or reached by 40 days following vaccination. In an embodiment of the invention, the antibody titer is produced or reached by 50 or more days following vaccination. In an embodiment of the invention, the antibody titer is produced or reached by 21 to 35 days following vaccination. In exemplary embodiments, the titer is produced or reached following a single dose of vaccine administered to the subject. In other embodiments, the titer is produced or reached following multiple doses, e.g., following a first and a second dose (e.g., a booster dose.). In exemplary embodiments, the titer is produced or reached following three doses of vaccine administered to the subject. In exemplary aspects of the invention, antigen-specific antibodies are measured in units of μg/ml or are measured in units of IU/L (International Units per liter) or mIU/ml (milli International Units per ml). In exemplary embodiments of the invention, an efficacious vaccine produces >0.05 μg/ml, >0.1 μg/ml, >0.2 μg/ml, >0.35 μg/ml, >0.5 μg/ml, >1 μg/ml, >2 μg/ml, >5 μg/ml or >10 μg/ml. In an embodiment of the invention, an efficacious vaccine produces >0.05 μg/ml of antigen-specific antibodies. In an embodiment of the invention, an efficacious vaccine produces >0.1 μg/ml of antigen-specific antibodies. In an embodiment of the invention, an efficacious vaccine produces >0.2 μg/ml of antigen-specific antibodies. In an embodiment of the invention, an efficacious vaccine produces >0.35 μg/ml of antigen-specific antibodies. In an embodiment of the invention, an efficacious vaccine produces >0.5 μg/ml of antigen-specific antibodies. In an embodiment of the invention, an efficacious vaccine produces >1 μg/ml of antigen-specific antibodies. In an embodiment of the invention, an efficacious vaccine produces >2 μg/ml of antigen-specific antibodies. In an embodiment of the invention, an efficacious vaccine produces >5 μg/ml of antigen-specific antibodies. In an embodiment of the invention, an efficacious vaccine produces >10 μg/ml of antigen-specific antibodies.

In exemplary embodiments of the invention, an efficacious vaccine produces >10 mlU/ml, >20 mlU/ml, >50 mlU/ml, >100 mlU/ml, >200 mlU/ml, >500 mlU/ml or >1000 mlU/ml. In an embodiment of the invention, an efficacious vaccine produces >10 mlU/ml. In an embodiment of the invention, an efficacious vaccine produces >20 mlU/ml. In an embodiment of the invention, an efficacious vaccine produces >50 mlU/ml. In an embodiment of the invention, an efficacious vaccine produces >100 mlU/ml. In an embodiment of the invention, an efficacious vaccine produces >200 mlU/ml. In an embodiment of the invention, an efficacious vaccine produces >500 mlU/ml or >1000 mlU/ml. In exemplary embodiments, the antibody level or concentration is produced or reached by 10 days following vaccination, by 20 days following vaccination, by 30 days following vaccination, by 40 days following vaccination, or by 50 or more days following vaccination. In an embodiment of the invention, the antibody level or concentration is produced or reached by 10 days following vaccination. In an embodiment of the invention, the antibody level or concentration is produced or reached by 20 days following vaccination. In an embodiment of the invention, the antibody level or concentration is produced or reached by 30 days following vaccination. In an embodiment of the invention, the antibody level or concentration is produced or reached by 40 days following vaccination. In an embodiment of the invention, the antibody level or concentration is produced or reached by 50 days following vaccination. In an embodiment of the invention, the antibody level or concentration is produced or reached by by 21 to 35 days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, the titer is produced or reached following three doses of vaccine administered to the subject. In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

In another embodiment, the immunoprotective response against SARS-COV-2 may be measured by CD4+ and CD8+ T-cell responses against SARS-COV-2 S protein and epitopes thereof. Functionality and polarization of S-specific SARS-COV-2 T cells induced by the mRNA composition may be assessed by intracellular accumulation of cytokines IFNγ, IL-2, and IL-4 measured after stimulation with overlapping peptide pools representing the full-length sequence of the whole SARS-COV-2 S protein.

For example, in a clinical trial conducted in 2020, described in the FDA Briefing Document for the Vaccines and Related Biological Products Advisory Committee Meeting, dated Dec. 10, 2020, most participants who received both doses of BNT162b2 had evidence of SARS-COV-2 S protein-specific CD4+(39/39, 100%) and CD8+ (35/39, 89.7%) T cell responses. These T cell responses were directed against different parts of the antigen, including epitopes in the RBD, indicating the induction of multi-epitope responses by BNT162b2. Functionality and polarization of S-specific BNT162b2-induced SARS-COV-2 T cells were assessed by intracellular accumulation of cytokines IFNγ, IL-2, and IL-4 measured after stimulation with overlapping peptide pools representing the full-length sequence of the whole SARS-COV-2 S protein. For benchmarking, PBMC fractions from 15 convalescent patients with virologically confirmed COVID-19 were used. The Th1 polarization of the T helper response was characterized by the IFNγ and IL-2 production, and only minor IL-4, production upon antigen-specific (SARS-COV-2 S protein peptide pools) re-stimulation. The SARS-COV-2 neutralizing geometric mean titer (GMTs) increased over baseline after Dose 1, with a boost effect after Dose 2 that was most pronounced at the 30 μg dose level. Thus, the immunogenicity results from Study BNT162-01 showed evidence of antibody-mediated SARS-COV-2 neutralization and a Th1 polarization in the cell-mediated cellular immune responses in healthy adults 18 to 55 years of age, which supports the final dose selection and prospect of benefit for the enrollment of larger numbers of participants in Study C4591001.

In a preferred embodiment the immunoprotective response elicited by the PCV of the invention against S. pneumoniae is not decreased by co-administering (e.g. concomitantly or concurrently) a mRNA vaccine of the invention as compared to the administration of the PCV of the invention alone.

Thus, surprisingly, in these embodiments it has been found that the mRNA vaccine does not immunologically interfere with the patient's response to the PCV, preferably the Prevnar13®, the V114 or the 20vPnC (Prevnar20®) vaccine.

In a preferred embodiment the immunoprotective response elicited by the PCV of the invention against S. pneumoniae is increased by co-administering (e.g. concomitantly or concurrently) a mRNA vaccine of the invention as compared to the administration of the PCV of the invention alone.

Thus, surprisingly, in these embodiments it has been found that the mRNA vaccine immunologically enhances the patient's response to the PCV, preferably the Prevnar13®, the V114 or the 20vPnC (Prevnar20®) vaccine.

Preferably such increase is observed for at least one conjugate in a multivalent PCV of the invention. Preferably such increase is observed for at least 2, 3, 4, 5, 6, 7, 8, 9, 10, 11, 12 or 13 conjugates in a multivalent PCV of the invention. In some such embodiments, the immunoprotective response is increased for at least one conjugate of the PCV of the invention. In some embodiments, the immunoprotective response is increased for at least two conjugates of the PCV of the invention. In some embodiments, the immunoprotective response is increased for at least three conjugates of the PCV of the invention. In some embodiments, the immunoprotective response is increased for at least four conjugates of the PCV of the invention. In some embodiments, the immunoprotective response is increased for at least five conjugates of the PCV of the invention. In some embodiments, the immunoprotective response is increased for at least six conjugates of the PCV of the invention. In some embodiments, the immunoprotective response is increased for at least seven conjugates of the PCV of the invention. In some embodiments, the immunoprotective response is increased for at least eight conjugates of the PCV of the invention. In some embodiments, the immunoprotective response is increased for at least nine conjugates of the PCV of the invention. In some embodiments, the immunoprotective response is increased for at least ten conjugates of the PCV of the invention. In some embodiments, the immunoprotective response is increased for at least eleven conjugates of the PCV of the invention. In some embodiments, the immunoprotective response is increased for at least three conjugates of the PCV of the invention. In some embodiments, the immunoprotective response is increased for at least twelve conjugates of the PCV of the invention. In some embodiments, the immunoprotective response is increased for at least thirteen conjugates of the PCV of the invention. In some embodiments, the immunoprotective response is increased for at least fourteen conjugates of the PCV of the invention. In some embodiments, the immunoprotective response is increased for at least fifteen conjugates of the PCV of the invention. In some embodiments, the immunoprotective response is increased for at least sixteen conjugates of the PCV of the invention. In some embodiments, the immunoprotective response is increased for at least seventeen conjugates of the PCV of the invention. In some embodiments, the immunoprotective response is increased for at least eighteen conjugates of the PCV of the invention. In some embodiments, the immunoprotective response is increased for at least nineteen conjugates of the PCV of the invention. In some embodiments, the immunoprotective response is increased for twenty conjugates of the PCV of the invention.

Preferably such increase is observed for all conjugates of a respective multivalent PCV. For example, in case of a 13-valent PCV (such as Prevnar13®), the immunoprotective response is increased for the thirteen conjugates of the PCV.

For example, in case of a 15-valent PCV (such as V114), the immunoprotective response is increased for the fifteen conjugates of the PCV.

For example, in case of a 20-valent PCV (such as 20vPnC (Prevnar20®)), the immunoprotective response is increased for the twenty conjugates of the PCV.

Preferably, such increase is at least 1.2-fold. In an embodiment, such increase is at least 1.3-fold. In an embodiment, such increase is at least 1.4-fold. In an embodiment, such increase is at least 1.5-fold. In an embodiment, such increase is at least 1.6-fold. In an embodiment, such increase is at least 1.7-fold. In an embodiment, such increase is at least 1.8-fold. In an embodiment, such increase is at least 1.9-fold. In an embodiment, such increase is at least 2-fold.

In an embodiment said increase is an increase of the IgG level.

In an embodiment said increase is an increase of the fold rise in IgG level from before to after vaccination.

In an embodiment said increase is an increase of the OPA titer.

In an embodiment said increase is an increase of the fold rise in OPA titers from before to after vaccination (1 month after vaccination).

Preferably such increase of the invention is statistically significant at a p-value less than 0.05.

In a preferred embodiment the immunoprotective response elicited by a mRNA vaccine of the invention against SARS-COV-2 is not decreased by co-administering (e.g. concomitantly or concurrently) a PCV vaccine of the invention as compared to the administration of the mRNA vaccine of the invention alone.

Thus, surprisingly, in these embodiments it has been found that the PCV does not immunologically interfere with the patient's response to the mRNA vaccine, preferably the BNT162b2 vaccine.

In a preferred embodiment the immunoprotective response elicited by a mRNA vaccine of the invention against SARS-COV-2 is increased by co-administering (e.g. concomitantly or concurrently) a PCV vaccine of the invention as compared to the administration of the mRNA vaccine of the invention alone.

Thus, surprisingly, in these embodiments it has been found that the PCV immunologically enhances the patient's response to the mRNA vaccine, preferably the BNT162b2 vaccine.

Preferably, such increase is at least 1.2-fold, 1.3-fold, 1.4-fold, 1.5-fold, 1.6-fold, 1.7-fold, 1.8-fold, 1.9-fold or 2-fold of the neutralizing antibody titer. In an embodiment, such increase is at least 1.2-fold, of the neutralizing antibody titer. In an embodiment, such increase is at least 1.3-fold, of the neutralizing antibody titer. In an embodiment, such increase is at least 1.4-fold, of the neutralizing antibody titer. In an embodiment, such increase is at least 1.5-fold, of the neutralizing antibody titer. In an embodiment, such increase is at least 1.6-fold, of the neutralizing antibody titer. In an embodiment, such increase is at least 1.7-fold, of the neutralizing antibody titer. In an embodiment, such increase is at least 1.8-fold, of the neutralizing antibody titer. In an embodiment, such increase is at least 1.9-fold, of the neutralizing antibody titer. In an embodiment, such increase is at least 2-fold, of the neutralizing antibody titer.

Preferably, such increase is at least 1.2-fold, 1.3-fold, 1.4-fold, 1.5-fold, 1.6-fold, 1.7-fold, 1.8-fold, 1.9-fold or 2-fold of antigen-specific (S-binding) antibody titer. In an embodiment, such increase is at least 1.2-fold of the neutralizing antibody titer. In an embodiment, such increase is at least 1.3-fold, of antigen-specific (S-binding) antibody titer. In an embodiment, such increase is at least 1.4-fold of antigen-specific (S-binding) antibody titer. In an embodiment, such increase is at least 1.5-fold of antigen-specific (S-binding) antibody titer. In an embodiment, such increase is at least 1.6-fold of antigen-specific (S-binding) antibody titer. In an embodiment, such increase is at least 1.7-fold of antigen-specific (S-binding) antibody titer. In an embodiment, such increase is at least 1.8-fold of antigen-specific (S-binding) antibody titer. In an embodiment, such increase is at least 1.9-fold of antigen-specific (S-binding) antibody titer. In an embodiment, such increase is at least 2-fold of antigen-specific (S-binding) antibody titer.

Preferably such increase of the invention is statistically significant a p-value less than 0.05.

In an embodiment, said pneumococcal conjugate vaccine and said mRNA vaccine against SARS-CoV-2 are administered concurrently.

In an embodiment, said pneumococcal conjugate vaccine and said mRNA vaccine against SARS-CoV-2 are administered concomitantly.

By “concurrent administration” is meant the administration of therapeutically effective doses of a first and a second immunogenic compositions through the same access site, but in separate unit dosage forms, within a short period of one another. Concurrent administration is essentially administering the two immunogenic compositions at about the same time but in separate dosage forms, through the same access site. The concurrent administration of the first and the second immunogenic compositions often occurs during the same physician office visit.

By “concomitant administration” is meant the administration of therapeutically effective doses of a first and a second immunogenic compositions, in separate unit dosage forms within a short period of one another at different anatomic sites. Concomitant administration is essentially administering the two immunogenic compositions at about the same time but in separate dosage forms and at different anatomic sites. The concomitant administration of the first and second immunogenic compositions often occurs during the same physician office visit.

In some cases, as little as one dose of each of the vaccines according to the invention is administered. In some circumstances however, a second, third or fourth dose may be given. Following an initial vaccination, subjects can receive one or several booster immunizations adequately spaced.

In an embodiment of the method of the invention, at least 2 doses of mRNA vaccine against SARS-COV-2 is administered. In an embodiment of the method of the invention, at least 3 doses of mRNA vaccine against SARS-COV-2 is administered. In an embodiment of the method of the invention, at least 4 doses of mRNA vaccine against SARS-COV-2 is administered.

In an embodiment of the method of the invention, 2 doses of mRNA vaccine against SARS-COV-2 is administered. In an embodiment of the method of the invention, 3 doses of mRNA vaccine against SARS-COV-2 is administered. In an embodiment of the method of the invention, 4 doses of mRNA vaccine against SARS-COV-2 is administered. In said embodiments, the doses of mRNA vaccine against SARS-COV-2 can be separated by an interval of about 2 weeks to about 12 months.

In an embodiment of the method of the invention, one dose of pneumococcal conjugate vaccine is administered.

In an embodiment of the method of the invention, at least 2 doses of pneumococcal conjugate vaccine is administered. In an embodiment of the method of the invention, at least 3 doses of pneumococcal conjugate vaccine is administered. In an embodiment of the method of the invention, at least 4 doses of pneumococcal conjugate vaccine is administered.

In an embodiment of the method of the invention, 2 doses of pneumococcal conjugate vaccine is administered. In an embodiment of the method of the invention, 3 doses of pneumococcal conjugate vaccine is administered. In an embodiment of the method of the invention, 4 doses of pneumococcal conjugate vaccine is administered. In said embodiments, said doses of pneumococcal conjugate vaccine can be separated by an interval of about 2 weeks to about 12 months.

In an embodiment of the method of the invention, 2 doses of mRNA vaccine against SARS-COV-2 and one dose of pneumococcal conjugate vaccine are administered. In said embodiments, said pneumococcal conjugate vaccine can be co-administered with the first dose of mRNA vaccine against SARS-COV-2. In another embodiment, said pneumococcal conjugate vaccine can be co-administered with the second dose of mRNA vaccine against SARS-COV-2. In another embodiment, said pneumococcal conjugate vaccine can be concomitantly administered with the first dose of mRNA vaccine against SARS-COV-2. In yet other embodiment, said pneumococcal conjugate vaccine can be concomitantly administered with the second dose of mRNA vaccine against SARS-COV-2. In other embodiments, said pneumococcal conjugate vaccine can be concurrently administered with the first dose of mRNA vaccine against SARS-COV-2. In yet further embodiments, said pneumococcal conjugate vaccine can be concurrently administered with the second dose of mRNA vaccine against SARS-COV-2.

In an embodiment of the method of the invention, 3 doses of mRNA vaccine against SARS-CoV-2 and one dose of pneumococcal conjugate vaccine are administered. In said embodiments, said pneumococcal conjugate vaccine can be co-administered with the first dose of mRNA vaccine against SARS-COV-2. In other embodiments, said pneumococcal conjugate vaccine can be co-administered with the second dose of mRNA vaccine against SARS-COV-2. In yet further embodiments, said pneumococcal conjugate vaccine is co-administered with the third dose of mRNA vaccine against SARS-COV-2. In other embodiments, said pneumococcal conjugate vaccine can be concurrently administered with the first dose of mRNA vaccine against SARS-CoV-2. In other embodiments, said pneumococcal conjugate vaccine can be concurrently administered with the second dose of mRNA vaccine against SARS-COV-2. In other embodiments, said pneumococcal conjugate vaccine can be concurrently administered with the third dose of mRNA vaccine against SARS-COV-2. In other embodiments, said pneumococcal conjugate vaccine can be concomitantly administered with the first dose of mRNA vaccine against SARS-COV-2. In other embodiments, said pneumococcal conjugate vaccine can be concomitantly administered with the second dose of mRNA vaccine against SARS-COV-2. In other embodiments, said pneumococcal conjugate vaccine can be concomitantly administered with the third dose of mRNA vaccine against SARS-COV-2.

In an embodiment of the method of the invention, 4 doses of mRNA vaccine against SARS-COV-2 and one dose of pneumococcal conjugate vaccine are administered. In said embodiments, said pneumococcal conjugate vaccine can be co-administered with the first dose of mRNA vaccine against SARS-COV-2. In other embodiments, said pneumococcal conjugate vaccine can be co-administered with the second dose of mRNA vaccine against SARS-COV-2. In other embodiments, said pneumococcal conjugate vaccine can be co-administered with the third dose of mRNA vaccine against SARS-COV-2. In other embodiments, said pneumococcal conjugate vaccine can be co-administered with the fourth dose of mRNA vaccine against SARS-COV-2. In other embodiments, said pneumococcal conjugate vaccine can be concurrently administered with the first dose of mRNA vaccine against SARS-COV-2. In other embodiments, said pneumococcal conjugate vaccine can be concurrently administered with the second dose of mRNA vaccine against SARS-COV-2. In other embodiments, said pneumococcal conjugate vaccine can be concurrently administered with the third dose of mRNA vaccine against SARS-COV-2. In other embodiments, said pneumococcal conjugate vaccine can be concurrently administered with the fourth dose of mRNA vaccine against SARS-COV-2. In other embodiments, said pneumococcal conjugate vaccine can be concomitantly administered with the first dose of mRNA vaccine against SARS-COV-2. In other embodiments, said pneumococcal conjugate vaccine can be concomitantly administered with the second dose of mRNA vaccine against SARS-COV-2. In other embodiments, said pneumococcal conjugate vaccine can be concomitantly administered with the third dose of mRNA vaccine against SARS-COV-2. In other embodiments, said pneumococcal conjugate vaccine can be concomitantly administered with the fourth dose of mRNA vaccine against SARS-COV-2.

In an embodiment of the method of the invention, 2 doses of mRNA vaccine against SARS-COV-2 and 2 doses of pneumococcal conjugate vaccine are administered.

In an embodiment of the method of the invention, 3 doses of mRNA vaccine against SARS-COV-2 and 2 doses of pneumococcal conjugate vaccine are administered.

In an embodiment of the method of the invention, 4 doses of mRNA vaccine against SARS-COV-2 and 2 doses of pneumococcal conjugate vaccine are administered.

In an embodiment of the method of the invention, if more than one dose of mRNA vaccine against SARS-COV-2 is administered, the first 2 doses of mRNA vaccine against SARS-COV-2 can be separated by an interval of about 2 weeks to about 6 months.

In an embodiment of the method of the invention, if more than one dose of mRNA vaccine against SARS-COV-2 is administered, the first 2 doses of mRNA vaccine against SARS-COV-2 can be separated by an interval of about 2 weeks to about 2 months.

In an embodiment of the method of the invention, if more than one dose of mRNA vaccine against SARS-COV-2 is administered, the first 2 doses of mRNA vaccine against SARS-COV-2 can be separated by an interval of about 2 weeks to about 6 weeks.

In an embodiment of the method of the invention, if more than one dose of mRNA vaccine against SARS-COV-2 is administered, the first 2 doses of mRNA vaccine against SARS-COV-2 can be separated by an interval of about 2 weeks to about 4 months.

In an embodiment of the method of the invention, if more than one dose of mRNA vaccine against SARS-COV-2 is administered, the first 2 doses of mRNA vaccine against SARS-COV-2 can be separated by an interval of about 3 weeks.

In an embodiment of the method of the invention, if three doses of mRNA vaccine against SARS-CoV-2 are administered, the first 2 doses of mRNA vaccine against SARS-COV-2 can be separated by an interval of about 2 weeks to about 6 months and the third dose can be separated from the second dose by an interval of at least about 6 months.

In an embodiment of the method of the invention, if three doses of mRNA vaccine against SARS-CoV-2 are administered, the first 2 doses of mRNA vaccine against SARS-COV-2 can be separated by an interval of about 2 weeks to about 2 months and the third dose can be separated from the second dose by an interval of at least about 6 months.

In an embodiment of the method of the invention, if three doses of mRNA vaccine against SARS-CoV-2 are administered, the first 2 doses of mRNA vaccine against SARS-COV-2 can be separated by an interval of about 2 weeks to about 6 weeks and the third dose can be separated from the second dose by an interval of at least about 6 months.

In an embodiment of the method of the invention, if three doses of mRNA vaccine against SARS-CoV-2 are administered, the first 2 doses of mRNA vaccine against SARS-COV-2 can be separated by an interval of about 2 weeks to about 4 months and the third dose can be separated from the second dose by an interval of at least about 6 months.

In an embodiment of the method of the invention, if three doses of mRNA vaccine against SARS-CoV-2 are administered, the first 2 doses of mRNA vaccine against SARS-COV-2 can be separated by an interval of about 3 weeks and the third dose can be separated from the second dose by an interval of at least about 6 months.

In an embodiment of the method of the invention, if three doses of mRNA vaccine against SARS-CoV-2 are administered, the first 2 doses of mRNA vaccine against SARS-COV-2 can be separated by an interval of about 2 weeks to about 6 months and the third dose can be separated from the second dose by an interval of at least about a year. 30)

In an embodiment of the method of the invention, if three doses of mRNA vaccine against SARS-CoV-2 are administered, the first 2 doses of mRNA vaccine against SARS-COV-2 can be separated by an interval of about 2 weeks to about 2 months and the third dose can be separated from the second dose by an interval of at least about a year.

In an embodiment of the method of the invention, if three doses of mRNA vaccine against SARS-CoV-2 are administered, the first 2 doses of mRNA vaccine against SARS-COV-2 can be separated by an interval of about 2 weeks to about 6 weeks and the third dose can be separated from the second dose by an interval of at least about a year.

In an embodiment of the method of the invention, if three doses of mRNA vaccine against SARS-CoV-2 are administered, the first 2 doses of mRNA vaccine against SARS-COV-2 can be separated by an interval of about 2 weeks to about 4 months and the third dose can be separated from the second dose by an interval of at least about a year.

In an embodiment of the method of the invention, if three doses of mRNA vaccine against SARS-CoV-2 are administered, the first 2 doses of mRNA vaccine against SARS-COV-2 can be separated by an interval of about 3 weeks and the third dose can be separated from the second dose by an interval of at least about a year.

In an embodiment of the method of the invention, the human subject has already received at least one mRNA vaccine dose against SARS-COV-2 prior to said co-administration. In an embodiment, said at least one mRNA vaccine dose against SARS-COV-2 has been administered at least about 3 weeks prior to said co-administration. In an embodiment, said at least one mRNA vaccine dose against SARS-COV-2 has been administered at least about 2 months prior to said co-administration. In an embodiment, said at least one mRNA vaccine dose against SARS-COV-2 has been administered at least about 6 months prior to said co-administration. In an embodiment, said at least one mRNA vaccine dose against SARS-COV-2 has been administered at least about one year prior to said co-administration. In an embodiment, said at least one mRNA vaccine dose against SARS-COV-2 has been administered at least about two years prior to said co-administration.

In an embodiment of the method of the invention, the human subject has already received one mRNA vaccine dose against SARS-COV-2 prior to said co-administration. In an embodiment, said one mRNA vaccine dose against SARS-COV-2 has been administered at least about 3 weeks prior to said co-administration. In an embodiment, said one mRNA vaccine dose against SARS-CoV-2 has been administered at least about 2 months prior to said co-administration. In an embodiment, said one mRNA vaccine dose against SARS-COV-2 has been administered at least about 6 months prior to said co-administration. In an embodiment, said one mRNA vaccine dose against SARS-COV-2 has been administered at least about one year prior to said co-administration. In an embodiment, said one mRNA vaccine dose against SARS-COV-2 has been administered at least about two years prior to said co-administration. In an embodiment, said human subject has already received at least two mRNA vaccine doses against SARS-COV-2 prior to said co-administration. In said embodiment, the last of said at least two mRNA vaccine doses against SARS-COV-2 has been administered at least about 3 weeks prior to said co-administration. In an embodiment, said human subject has already received at least two mRNA vaccine doses against SARS-COV-2 prior to said co-administration wherein the last of said at least two mRNA vaccine doses against SARS-COV-2 has been administered at least about 2 months prior to said co-administration. In an embodiment, said human subject has already received at least two mRNA vaccine doses against SARS-COV-2 prior to said co-administration wherein the last of said at least two mRNA vaccine doses against SARS-COV-2 has been administered at least about 6 months prior to said co-administration. In an embodiment, said human subject has already received at least two mRNA vaccine doses against SARS-COV-2 prior to said co-administration wherein the last of said at least two mRNA vaccine doses against SARS-CoV-2 has been administered at least about one year prior to said co-administration. In an embodiment, said human subject has already received at least two mRNA vaccine doses against SARS-COV-2 prior to said co-administration wherein the last of said at least two mRNA vaccine doses against SARS-COV-2 has been administered at least about two years prior to said co-administration.

In an embodiment of the method of the invention, the human subject has already received two mRNA vaccine doses against SARS-COV-2 prior to said co-administration. In an embodiment, the last of said two mRNA vaccine doses against SARS-COV-2 has been administered at least about 3 weeks prior to said co-administration. In an embodiment of the method of the invention, the human subject has already received two mRNA vaccine doses against SARS-COV-2 prior to said co-administration wherein the last of said two mRNA vaccine doses against SARS-COV-2 has been administered at least about 2 months prior to said co-administration. In an embodiment of the method of the invention, the human subject has already received two mRNA vaccine doses against SARS-COV-2 prior to said co-administration wherein the last of said two mRNA vaccine doses against SARS-COV-2 has been administered at least about 6 months prior to said co-administration. In an embodiment of the method of the invention, the human subject has already received two mRNA vaccine doses against SARS-COV-2 prior to said co-administration wherein the last of said two mRNA vaccine doses against SARS-COV-2 has been administered at least about one year prior to said co-administration. In an embodiment of the method of the invention, the human subject has already received two mRNA vaccine doses against SARS-COV-2 prior to said co-administration wherein the last of said two mRNA vaccine doses against SARS-COV-2 has been administered at least about two years prior to said co-administration.

In an embodiment of the method of the invention, said co-administration is a booster dose of said mRNA vaccine against SARS-COV-2.

In an embodiment the invention relates to a pneumococcal conjugate vaccine and an mRNA vaccine against SARS-COV-2 for use in a method for eliciting an immunoprotective response in a human subject against S. pneumoniae and Severe Acute Respiratory Syndrome Coronavirus 2 (SARS-COV-2), said method comprising co-administering to the human subject said vaccines. In an embodiment the invention relates to a pneumococcal conjugate vaccine and an mRNA vaccine against SARS-COV-2 for use in a method for eliciting an immunoprotective response in a human subject against S. pneumoniae and Severe Acute Respiratory Syndrome Coronavirus 2 (SARS-COV-2), said method comprising co-administering to the human subject said vaccines wherein said pneumococcal conjugate vaccine and said mRNA vaccine against SARS-COV-2 are administered concomitantly.

In an embodiment the invention relates to the use of the co-administration of a pneumococcal conjugate vaccine and of an mRNA vaccine against SARS-COV-2 as a booster dose of an mRNA vaccine against SARS-COV-2.

In an embodiment the invention relates to the use of the co-administration of a pneumococcal conjugate vaccine and of an mRNA vaccine against SARS-COV-2 as a booster dose of said mRNA vaccine against SARS-COV-2.

In an embodiment the invention relates to the use of the co-administration of a pneumococcal conjugate vaccine and of an mRNA vaccine against SARS-COV-2 for boosting an mRNA vaccine against SARS-COV-2.

In an embodiment the invention relates to the use of the co-administration of a pneumococcal conjugate vaccine and of an mRNA vaccine against SARS-COV-2 for boosting said mRNA vaccine against SARS-COV-2.

In an embodiment the invention relates to the co-administration of a pneumococcal conjugate vaccine and of an mRNA vaccine against SARS-COV-2 for use as a booster dose of an mRNA vaccine against SARS-COV-2.

In an embodiment the invention relates to the co-administration of a pneumococcal conjugate vaccine and of an mRNA vaccine against SARS-COV-2 for use as a booster dose of said mRNA vaccine against SARS-COV-2.

In an embodiment the invention relates to the co-administration of a pneumococcal conjugate vaccine and of an mRNA vaccine against SARS-COV-2 for use in a method of boostering an mRNA vaccine against SARS-COV-2.

In an embodiment the invention relates to the co-administration of a pneumococcal conjugate vaccine and of an mRNA vaccine against SARS-COV-2 for use in a method of boostering said mRNA vaccine against SARS-COV-2.

Said pneumococcal conjugate vaccine and said mRNA vaccine against SARS-COV-2 can be as disclosed herein.

In an embodiment, the vaccines disclosed herein are administered by intramuscular or subcutaneous injection. In an embodiment, the vaccines disclosed herein are administered by intramuscular injection. In an embodiment, the vaccines disclosed herein are administered by subcutaneous injection.

In an embodiment, the vaccines are administered by intramuscular injection in a thigh or arm. In an embodiment, the injection site is the anterolateral thigh muscle or the deltoid muscle. In an embodiment, the vaccines are administered via intramuscular injection to the deltoid muscle of an arm.

In an embodiment, the vaccines are administered by subcutaneous injection in a thigh or an arm. In an embodiment, the injection site is the fatty tissue over the anterolateral thigh muscle or the fatty tissue over triceps.

In case of concomitant administration, the first injection can be made in one thigh and the second in the other thigh (preferably in the anterolateral thigh muscles). Alternatively, the first injection can be made in one arm and the second in the other arm (preferably in the deltoid muscles). The first injection can also be made in a thigh and the second in an arm or the first injection in an arm and the second in a thigh. In case of concomitant administration, the vaccines are preferably administered via intramuscular injection to the deltoid muscle of each arm.

7. Subject to be Treated with the Method of the Invention

As disclosed herein, the vaccines described herein may be used for eliciting an immunoprotective response in a human against S. pneumoniae and Severe Acute Respiratory Syndrome Coronavirus 2 (SARS-COV-2).

In an embodiment of the present invention, the human subject to be co-administered a pneumococcal conjugate vaccine and a mRNA vaccine against SARS-COV-2 is a human adult 50 years of age or older. More preferably the human subject is a human adult 60 years of age or older. Even more preferably, the human subject is a human adult 65 years of age or older. In an embodiment, the human subject is 70 years of age or older, 75 years of age or older or 80 years of age or older.

In an embodiment the human subject to be co-administered a pneumococcal conjugate vaccine and a mRNA vaccine against SARS-COV-2 is an immunocompromised individual. An immunocompromised individual is generally defined as a person who exhibits an attenuated or reduced ability to mount a normal humoral or cellular defense to challenge by infectious agents. In an embodiment of the present invention, the immunocompromised human to be co-administered a pneumococcal conjugate vaccine and a mRNA vaccine against SARS-COV-2 suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised human subject to be co-administered a pneumococcal conjugate vaccine and a mRNA vaccine against SARS-COV-2 suffers from a disease selected from the group consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised human subject to be co-administered a pneumococcal conjugate vaccine and a mRNA vaccine against SARS-COV-2 suffers from malnutrition.

In a particular embodiment of the present invention, the immunocompromised human subject to be co-administered a pneumococcal conjugate vaccine and a mRNA vaccine against SARS-COV-2 is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised human subject to be co-administered a pneumococcal conjugate vaccine and a mRNA vaccine against SARS-COV-2 is a smoker.

In a particular embodiment of the present invention, the immunocompromised human subject to be co-administered a pneumococcal conjugate vaccine and a mRNA vaccine against SARS-COV-2 has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised human subject to be co-administered a pneumococcal conjugate vaccine and a mRNA vaccine against SARS-COV-2 suffers from neutropenia. In a particular embodiment of the present invention, the immunocompromised human subject to be co-administered a pneumococcal conjugate vaccine and a mRNA vaccine against SARS-COV-2 has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy. Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised human subjects disclosed herein is a human male or a human female.

In an embodiment of the present invention, the human subject to be co-administered a pneumococcal conjugate vaccine and a mRNA vaccine against SARS-COV-2 has already received at least one mRNA vaccine dose against SARS-COV-2 prior to said co-administration. Preferably, said at least one mRNA vaccine dose against SARS-COV-2 is a dose of BNT162b2.

In an embodiment of the present invention, the human subject to be co-administered a pneumococcal conjugate vaccine (PCV) and a mRNA vaccine against SARS-COV-2 has already received at least two mRNA vaccine doses against SARS-COV-2 prior to said co-administration. Preferably, said at least two mRNA vaccine doses against SARS-COV-2 each are a dose of BNT162b2. In a preferred embodiment said PCV co-administered with said mRNA vaccine against SARS-COV-2 is Prevnar13®, V114 or the 20vPnC (Prevnar20®) vaccine.

Accordingly, it is a preferred embodiment of the present invention to co-administer a dose of a PCV with a booster dose of a mRNA vaccine against SARS-COV-2. In a preferred embodiment said PCV co-administered with said booster dose is Prevnar13®, V114 or the 20vPnC (Prevnar20®) vaccine.

### EXAMPLE

### Example 1. Safety and Immunogenicity Study of a 20-Valent Pneumococcal Conjugate Vaccine (20vPnC) when Coadministered with an mRNA Vaccine to Prevent Infection with SARS-COV-2 (BNT162b2)

Because of the current lack of safety and immunogenicity data on COVID-19 vaccines coadministered with other vaccines, current US Advisory Committee on immunization Practices (ACIP) guidance is that COVID-19 vaccines should be administered alone, with a minimum interval of 14 days before or after administration of any other vaccine.

A clinical study has been designed to describe the safety and immunogenicity of a 20-valent Pneumococcal Conjugate Vaccine (20vPnC) and a booster dose of BNT162b2 (an mRNA vaccine to prevent infection with SARS-COV-2) when administered together at the same visit compared to each of the vaccines given alone in adults ≥65 years of age, as shown in FIG. 1.

### Objectives:

### Primary Objective:

To describe the safety profile of 20vPnC and a booster dose of BNT162b2 when coadministered or administered alone.

### Secondary Objectives:

To describe the immune response elicited by 20vPnC when coadministered with a booster dose of BNT162b2 or when administered alone.
To describe the immune response elicited by a booster dose of BNT162b2 when coadministered with 20vPnC or when administered alone.

### Endpoints:

### Primary (Safety):

Prompted local reactions at each injection site (redness, swelling, and pain at the injection site)
Prompted systemic events (fever, headache, chills, fatigue, muscle pain, and joint pain)
Adverse Events
Serious Adverse Events

### Secondary:

Pneumococcal Immunogenicity: Pneumococcal OPA titers OPA GMTs approximately 1 month after vaccination
BNT162b2 Immunogenicity: Full-length S-binding IgG levels

### Estimands:

### Primary (Safety):

In participants receiving at least 1 dose of study intervention and having safety follow-up after vaccination, the percentage of participants reporting:
Prompted local reactions at each injection site for up to 10 days following vaccination
Prompted systemic events for up to 7 days following vaccination
AEs from vaccination at Visit 1 through approximately 1 month after vaccination
SAEs from vaccination at Visit 1 through 6 months after vaccination

### Secondary:

Pneumococcal Immunogenicity: In participants in compliance with the key protocol criteria (evaluable participants): OPA GMTs (Geometric Mean Titers) approximately 1 month after vaccination
BNT162b2 Immunogenicity: In evaluable participants: GMCs (Geometric Mean Concentration) of full-length S-binding IgG levels approximately 1 month after vaccination, GMFR (Geometric Mean Fold Rise) in full-length S-binding IgG levels from before to approximately 1 month after vaccination

### Tertiary/Exploratory:

Pneumococcal Immunogenicity: In evaluable participants: GMFR in OPA titers from before to approximately 1 month after vaccination, The percentage of participants with a ≥4-fold rise in OPA titers from before to approximately 1 month after vaccination, The percentage of participants with OPA titers ≥LLOQ before vaccination and approximately 1 month after vaccination.
BNT162b2 Immunogenicity: In evaluable participants: GMTs of SARS-COV-2 reference-strain neutralizing titers approximately 1 month after vaccination, GMFRs in SARS-COV-2 reference-strain neutralizing titers from before to approximately 1 month after vaccination

### Overall Design

This is a Phase 3, multicenter, randomized, double-blind study conducted at investigator sites in the US. The purpose of this study is to describe the safety and immunogenicity of 20vPnC and a booster dose of BNT162b2 when administered together at the same visit compared to each of the vaccines given alone in adults ≥65 years of age, as shown in FIG. 1.

Approximately 600 participants from the US, 65 years of age and older who received 2 doses of 30 μg BNT162b2, are stratified by prior pneumococcal vaccine status (no previous pneumococcal vaccine [naïve] or receipt of at least 1 dose of a pneumococcal vaccine [experienced]) and randomized at a 1:1:1 ratio to 1 of 3 vaccine groups.

At Visit 1 (Day 1), the Coadministration group (20vPnC+BNT162b2) receives 20vPnC and a booster dose of BNT162b2, the 20vPnC-only group (20vPnC+saline) receives 20vPnC and saline, and the BNT162b2-only group (BNT162b2+saline) receives a booster dose of BNT162b2 and saline.

Participants from all groups have blood drawn at Visit 1 prior to vaccination, and at Visit 2, approximately 1 month after vaccination, for immunogenicity assessments and serological testing for prior COVID-19 infection.

### Number of Participants

Approximately 600 participants (200 per group) are randomly assigned to study intervention.

### Intervention Groups and Duration

Participants are randomized at a 1:1:1 ratio to 1 of 3 vaccine groups. At Visit 1 (Day 1), the Coadministration group receives 20vPnC and a booster dose of BNT162b2, the 20vPnC-only group receives 20vPnC and saline, and the BNT162b2-only group receives a booster dose of BNT162b2 and saline. Study intervention will be administered by an unblinded administrator via intramuscular injection to the upper deltoid muscle of each arm.

The duration of the study for each participant is approximately 6 months.

### Statistical Methods

Safety is evaluated by descriptive summary statistics (including counts and percentages of participants and the associated 2-sided 95% Cls) for local reactions at each injection site, systemic events, AEs, and SAEs for each vaccine group.

Pneumococcal immunogenicity is evaluated descriptively by OPA GMTs approximately 1 month after 20vPnC. Other assessments of immune response including OPA GMFRs from before to approximately 1 month after 20vPnC, percentages of participants with >4-fold rises in OPA titers from before to approximately 1 month after 20vPnC, and percentages of participants with OPA titers ≥LLOQ approximately 1 month after 20vPnC is also described, each with corresponding 95% Cls in evaluable participants from the Coadministration and 20vPnC-only groups.

BNT162b2 immunogenicity is evaluated descriptively using GMCs of full-length S-binding IgG levels approximately 1 month after BNT162b2, and GMFR from before to approximately 1 month after BNT162b2, each with corresponding 2-sided 95% Cls in evaluable participants from the Coadministration and BNT162b2-only groups.

### Dose

The 20vPnC candidate contains capsular polysaccharides from pneumococcal serotypes 1, 3, 4, 5, 6A, 6B, 7F, 8, 9V, 10A, 11A, 12F, 14, 15B, 18C, 19A, 19F, 22F, 23F, and 33F individually conjugated to CRM₁₉₇. The vaccine formulation contains 2.2 μg of each saccharide, except for 4.4 μg of 6B, per 0.5-mL dose (Intramuscular injection). In adults, administration of 1 dose of pneumococcal conjugate vaccine induces immune responses.

The modRNA BNT162b2 vaccine candidate is administered at a dose of 30 μg per 0.3-mL dose (Intramuscular injection). This is the dose that has shown to be efficacious and has been authorized for conditional or emergency use and is anticipated to be licensed in the future in the US.

For these products the term “dose” refers to an injection of a vaccine.

### Study Population

### Inclusion Criteria

Participants are eligible to be included in the study only if all of the following criteria apply:

### Age and Sex:

1. Male or female participants ≥65 years of age at the time of consent.

### Type of Participant and Disease Characteristics:

2. Participating or participated in Study C4591001, received 2 doses of 30 μg BNT162b2 with the second dose given ≥6 months prior to the first vaccination in this study, and have not received a third dose of BNT162b2.
3. Participants who are willing and able to comply with all scheduled visits, treatment plan, laboratory tests, lifestyle considerations, and other study procedures.
4. Participants who are determined by medical history, physical examination (if required), and clinical judgment of the investigator to be eligible for inclusion in the study.
5. Expected to be available for the duration of the study and can be contacted by telephone during study participation.
6. Male participant who is able to father children and willing to use an acceptable method of contraception; or female participant not of childbearing potential; or male participant not able to father children.
7. Adults who have no history of ever receiving a pneumococcal vaccine (ie, pneumococcal vaccine-naïve), or received a licensed pneumococcal vaccination ≥12 months prior to the first vaccination in this study.

### Informed Consent:

8. Capable of giving signed informed consent.

### Exclusion Criteria

Participants are excluded from the study if any of the following criteria apply:

### Medical Conditions:

1. History of severe adverse reaction associated with a vaccine and/or severe allergic reaction (eg, anaphylaxis) to any component of the study intervention(s) or any other diphtheria toxoid-containing vaccine.
2. Serious chronic disorder, including metastatic malignancy, severe COPD requiring supplemental oxygen, end-stage renal disease with or without dialysis, cirrhosis of the liver, clinically unstable cardiac disease, or any other disorder that in the investigator's opinion would make the participant inappropriate for entry into the study.
3. History of microbiologically proven invasive disease caused by S pneumoniae.
4. Previous clinical or microbiological diagnosis of COVID-19.
5. Known or suspected immunodeficiency (aside from stable HIV) or other conditions associated with immunosuppression, including, but not limited to, immunoglobulin class/subclass deficiencies, generalized malignancy, leukemia, lymphoma, or organ or bone marrow transplant.
6. Bleeding diathesis or condition associated with prolonged bleeding that would, in the opinion of the investigator, contraindicate intramuscular injection.
7. Congenital, functional, or surgical asplenia.
8. Current febrile illness (body temperature ≥100.4° F. [>38.0° C.]) or other acute illness within 48 hours before study intervention administration.
9. Other medical or psychiatric condition including recent (within the past year) or active suicidal ideation/behavior or laboratory abnormality that may increase the risk of study participation or, in the investigator's judgment, make the participant inappropriate for the study.

### Prior/Concomitant Therapy:

10. Previous vaccination with any investigational pneumococcal vaccine, or planned receipt of any licensed or investigational pneumococcal vaccine through study participation.
11. Previous vaccination with any coronavirus vaccine, other than those received in Study C4591001.
12. Currently receives treatment with immunosuppressive therapy, including cytotoxic agents or systemic corticosteroids, receipt of short-term (<14 days) systemic corticosteroids for treatment of an acute illness in the 28 days before administration of study intervention, or planned receipt through the last blood draw (Visit 2). Inhaled/nebulized, intra-articular, intrabursal, or topical (skin, eyes, or ears) corticosteroids are permitted.
13. Receipt of blood/plasma products, immunoglobulin, or monoclonal antibodies from 60 days before administration of study intervention, or receipt of any passive antibody therapy specific to COVID-19 from 90 days before administration of study intervention, or planned receipt through Visit 2.
14. Receipt of any inactivated or otherwise nonlive vaccine within 14 days or any live vaccine within 28 days before administration of study intervention.

### Prior/Concurrent Clinical Study Experience:

15. Participation in other studies involving investigational drugs, investigational vaccines, or investigational devices within 28 days prior to study entry and/or during study participation other than Study C4591001. Participation in purely observational studies is acceptable.
16. Previous participation in studies other than C4591001 involving study intervention containing LNPs.

### Administration

### Participants Receives Study Intervention at Visit 1.

At Visit 1, participants receives a single 0.5-mL dose of either 20vPnC or saline injected intramuscularly into the right deltoid, and a single 0.3-mL dose of either BNT162b2 or saline injected intramuscularly into the left deltoid.

### Immunogenicity Assessments

Blood samples are collected from all participants at Visits 1 and 2.

### Pneumococcal Responses

OPA titers for the 20vPnC serotypes are measured in sera collected at Visits 1 and 2 from the Coadministration and 20vPnC-only groups.

### BNT162b2 Responses

IgG levels are measured in the SARS-COV-2 full-length S-binding assay in sera collected at Visits 1 and 2 from the Coadministration and BNT162b2-only groups.

SARS-COV-2 reference-strain neutralizing titers may be measured in a subset of sera collected at Visits 1 and 2 from the Coadministration and BNT162b2-only groups.

Blood samples taken at Visits 1 and 2 are also measured for the N-binding antibody.

### Example 2: Safety, Tolerability, and Immunogenicity of a Booster Dose of BNT162b2 COVID-19 Vaccine Coadministered with 20-Valent Pneumococcal Conjugate Vaccine (PCV20) in Adults 65 Years of Age and Above

Introduction. Adults ≥65 years of age are at increased risk of morbidity and mortality from COVID-19 and from pneumococcal disease. The efficacy and safety of 2 doses (30 μg/dose administered 21 days apart) of the BNT162b2 mRNA COVID-19 vaccine (Comirnaty) in preventing Covid-19 were established in a global, randomised, placebo-controlled phase ⅔ trial (Study C4591001, NCT04368728) of individuals ≥16 years of age, with 94.7% efficacy in those ≥65 years of age. The 20-valent pneumococcal conjugate vaccine (PCV20), recently approved in the United States and Europe for the prevention of invasive pneumococcal disease and pneumonia due to vaccine serotypes in adults, contains the components of the 13-valent pneumococcal conjugate vaccine (PCV13) plus the conjugated polysaccharides of 7 additional serotypes; PCV13 has demonstrated efficacy and safety against pneumococcal pneumonia, including nonbacteremic pneumonia, in randomised controlled trials in adults ≥65 years of age. A booster dose of COVID-19 vaccine is now recommended for adults in many countries; thus, the BNT162b2 vaccine may be used in the same population as PCV20, as their target populations overlap.

### Study Design and Participants.

This phase 3, multicentre, randomised, double-blind study (ClinicalTrials.gov NCT04887948) was conducted in the United States from 20 May 2021 to 8 Dec. 2021.
Participants were adults ≥65 years of age who had received 2 doses of 30 μg BNT162b2 in the pivotal efficacy study (C4591001), with the second dose given ≥6 months before vaccination in this study, and who had not received a booster dose of any COVID-19 vaccine.
Participants were randomised 1:1:1 into 1 of 3 groups, stratified by prior pneumococcal vaccine status (naive or experienced), as follows: Coadministration group (both vaccines administered in opposite participant arms at the same visit), PCV20-only group, and BNT162b2-only group. In the control groups, saline was administered in the opposite participant arm to maintain blinding.

Three visits were performed in the study.
At Visit 1 (Day 1), participants were screened and enrolled, blood was drawn for immunogenicity testing, and vaccine was administered.
At Visit 2 (21-35 days after Visit 1), blood was drawn for immunogenicity testing, and safety data were collected.
At Visit 3 (approximately 6 months after Visit 1), participants were contacted by telephone to collect safety data.

### Safety:

Prompted local reactions (redness, swelling, pain at injection site) at each injection site and systemic events (fever, fatigue, headache, chills, muscle pain, joint pain) were assessed using an e-diary for 10 and 7 days after vaccination, respectively.
Adverse events (AEs) and serious AEs (SAEs) were collected for 1 month and 6 months after vaccination, respectively.

### Immunogenicity:

Serotype-specific opsonophagocytic activity (OPA) titres for the PCV20 serotypes were measured in the Pfizer OPA assay before and 1 month after vaccination in the 2 groups that received PCV20.
SARS-COV-2 full-length S-binding IgG concentrations were measured before and 1 month after vaccination in the 2 groups that received BNT162b2.

### Statistical Analyses:

The statistical analysis was descriptive, with no hypothesis testing.
Safety results were descriptively summarised for the safety population, which included all participants who received any study vaccination and had safety follow-up.
Immunogenicity results were descriptively summarised for the evaluable immunogenicity population, which included participants who were vaccinated as randomised, had at least one OPA titre or SARS-COV-2 full-length S-binding IgG concentration from a blood sample collected 1 month after vaccination, and had no major protocol deviations as determined by the clinician. Additionally, participants with clinically documented SARS-COV-2 infection occurring between vaccination and 1 month after BNT162b2 vaccination were excluded from the SARS-COV2 IgG analysis.
Serotype-specific OPA geometric mean titres (GMTs) and SARS-COV-2 full-length S-binding IgG geometric mean concentrations (GMCs) and the corresponding 2-sided 95% Cls were calculated by exponentiating the mean logarithm of the titres or concentrations and the corresponding confidence intervals (Cls; based on the Student's t distribution).
Geometric mean fold rises (GMFRs) in full-length S-binding IgG levels from before to approximately 1 month after vaccination (secondary endpoint) were calculated as the mean of the difference of logarithmically transformed assay results (later minus earlier) and exponentiated back to the original units. The associated 2-sided 95% Cls were computed by exponentiating the CIs using Student's t distribution for the mean difference on the natural log scale.
A post hoc analysis using a linear regression model was performed to compare serotype-specific OPA titres 1 month after vaccination in the Coadministration and PCV20-only groups. A similar post hoc analysis evaluated full-length S-binding concentrations 1 month after vaccination in the Coadministration group compared to the BNT162b2-only group.

### Study Population:

A total of 570 participants were randomised.
   559 participants were vaccinated and comprise the safety population (Coadministration, n=187; PCV20-only, n=187; BNT162b2-only, n=185).
Demographic characteristics were similar among the vaccine groups (Table 1).
The average time elapsed since the second dose of BNT162b2 was 8.1 months, and the average elapsed time since the most recent pneumococcal vaccination was 3.5 years (Table 1).

TABLE 1
Demographic and Baseline Characteristics (Safety Population)

| | Coadministration | PCV20 Only | BNT162b2 Only | |
|---|---|---|---|---|
| | (PCV20 + BNT162b2) | (PCV20 + Saline) | (BNT162b2 + Saline) | Total |
| | (N = 187) | (N = 187) | (N = 185) | (N = 559) |
| Sex, n (%) | | | | |
| Male | 98 (52.4) | 112 (59.9) | 108 (58.4) | 318 (56.8) |
| Female | 89 (47.6) | 75 (40.1) | 77 (41.6) | 241 (43.1) |
| Race, n (%) | | | | |
| White | 164 (87.7) | 172 (92.0) | 165 (89.2) | 501 (89.6) |
| Black or African American | 8 (4.3) | 7 (3.7) | 10 (5.4) | 25 (4.5) |
| Asian | 10 (5.3) | 5 (2.7) | 9 (4.9) | 24 (4.3) |
| Other | 5 (2.7) | 3 (1.6) | 1 (0.5) | 9 (1.6) |
| Ethnicity, n (%) | | | | |
| Hispanic/Latino | 28 (15.0) | 26 (13.9) | 25 (13.5) | 79 (14.1) |
| Non-Hispanic/non-Latino | 159 (85.0) | 161 (86.1) | 160 (86.5) | 480 (85.9) |
| Age group, n (%), y | | | | |
| 65 to 69 | 76 (40.6) | 69 (36.9) | 72 (38.9) | 217 (38.9) |
| 70 to 74 | 73 (39.0) | 69 (36.9) | 59 (31.9) | 201 (36.0) |
| 75 to 79 | 29 (15.5) | 34 (18.2) | 41 (22.2) | 104 (18.6) |
| ≥80 | 9 (4.8) | 15 (8.0) | 13 (7.0) | 37 (6.6) |
| Age at vaccination, y | | | | |
| Mean (SD) | 71.0 (4.22) | 71.8 (4.94) | 71.8 (4.95) | 71.5 (4.72) |
| Elapsed time since second dose of | | | | |
| BNT162b2, months | | | | |
| Mean (SD) | 8.2 (0.76) | 8.2 (0.66) | 8.0 (0.75) | 8.1 (0.73) |
| Elapsed time since prior | | | | |
| pneumococcal vaccination, years | | | | |
| Mean (SD) | 3.3 (1.44) | 3.7 (1.70) | 3.6 (1.55) | 3.5 (1.57) |

PCV20 = 20-valent pneumococcal conjugate vaccine; SD = standard deviation.
*Includes American Indian or Alaska Native, multiracial or not reported.

Safety. The percentages of participants who reported local reactions within 10 days at each of the PCV20 and BNT162b2 injection sites were similar regardless of whether the vaccines were given together or alone, and reactions were generally mild or moderate in severity. The rates of systemic events were similar in the Coadministration group and the BNT162b2-only group and were generally higher than those in the PCV20-only group; events were generally mild to moderate in severity.
Fatigue was the most frequently reported systemic event for all vaccine groups.

The rates of AEs within 1 month after vaccination and serious AEs (SAEs) through 6 months after vaccination were low and similar across all groups.

No SAEs were considered related to vaccine, and there was one death during the study (duodenal perforation, not considered related to vaccine).

PCV20 elicited robust immune responses to all 20 serotypes that were similar when

PCV20 was coadministered with BNT162b2 or given alone (FIG. 4).

The third BNT162b2 dose also elicited robust immune IgG responses to the SARS-COV-2 full-length S-binding protein, that were similar whether BNT162b2 was coadministered with PCV20 or given alone (FIG. 5).
The observed GMFR in full-length S-binding IgG levels from before to 1 month after the booster dose of BNT162b2 was similar in the coadministration and BNT162b2-only groups (35.5 and 39.0, respectively).

Based on post hoc analyses, the OPA GMRs of the Coadministration group to the PCV20-only group 1 month after PCV20 ranged from 0.77 (serotypes 8, 19F, and 23F) to 1.11 (serotype 19A; FIG. 6)

Full-length S-binding IgG GMR of the Coadministration group to the BNT162b2-only group 1 month after the booster dose of BNT162b2 was 1.06 (95% CI, 0.91, 1.23).

The responses would be statistically noninferior, with the lower bound of the 95% CI of the pneumococcal OPA GMR of coadministration to PCV20-only >0.5 for each serotype and >0.67 for the IgG GMR of coadministration to BNT162b2-only to the SARS-COV-2 full-length S-binding protein; 0.5 and 0.67 correspond to standard 2-fold and 1.5-fold noninferiority criteria, respectively, for these endpoints.

### Conclusion:

Coadministration of PCV20 and BNT162b2 was well tolerated, with an overall safety profile that was similar to BNT162b2 administered alone.
Robust immune responses were observed regardless of concomitant or separate administration of PCV20 and BNT162b2.

### Example 3: Safety, Tolerability, and Immunogenicity of a Booster (Third Dose) of BNT162b2 COVID-19 Vaccine Coadministered With 20-Valent Pneumococcal Conjugate Vaccine in Adults ≥65 Years Old

Background: Older adults are at increased risk of adverse outcomes from pneumococcal disease and COVID-19. Vaccination is an established strategy for preventing both illnesses. This study evaluated the safety and immunogenicity of coadministration of the 20-valent pneumococcal conjugate vaccine (PCV20) and a booster (third dose) of BNT162b2 COVID-19 vaccine.

Methods: This phase 3, randomized, double-blind, multicenter study included 570 participants aged ≥65 years randomized 1:1:1 to PCV20 and BNT162b2 coadministered, or PCV20 or BNT162b2 only (administered with saline for blinding). Primary safety endpoints included local reactions, systemic events, adverse events (AEs) and serious AEs (SAEs). Secondary objectives were immunogenicity of PCV20 and BNT162b2 when administered together or separately.

Results: Coadministration of PCV20 and BNT162b2 was well tolerated. Local reactions and systemic events were generally mild-moderate; injection-site pain and fatigue were the most frequent local and systemic events, respectively. AE and SAE rates were low and similar across groups. No AEs led to discontinuation; no SAEs were considered vaccination-related. Robust immune responses were observed, with opsonophagocytic activity geometric mean fold rises (GMFRs; from baseline to 1 month) of 2.5-24.5 and 2.3-30.6 across PCV20 serotypes in Coadministration and PCV20-only groups, respectively. GMFRs for full-length S-binding IgG of 35.5 and 39.0, and for neutralizing titers against SARS-COV-2-wild type virus of 58.8 and 65.4, were observed in the Coadministration and BNT162b2-only groups, respectively.

Conclusions: Safety and immunogenicity of coadministered PCV20 and BNT162b2 were similar to those of PCV20 or BNT162b2 administered alone, suggesting that the 2 vaccines may be coadministered.

### Methods

### Study Design and Participants

This phase 3, randomized, double-blind, multicenter study (ClinicalTrials.gov, NCT04887948), conducted in the United States between May 20-Dec. 8, 2021, evaluated the safety, tolerability, and immunogenicity of PCV20 when coadministered with a booster of BNT162b2 compared with individual administration. Eligible participants were >65 years old, healthy, or with stable preexisting disease (not requiring significant change in therapy or hospitalization for worsening disease <6 weeks before enrollment). Participants had received 2 doses of 30 μg BNT162b2 in the pivotal efficacy study (C4591001), with the second dose given >6 months before vaccination in this study, and had not received a booster dose of any COVID-19 vaccine.

Individuals who had received pneumococcal vaccination <12 months prior were excluded, as were those with a history of microbiologically proven invasive disease caused by S pneumoniae or a previous clinical or microbiological diagnosis of COVID-19. Additional exclusion criteria were a history of severe adverse reaction associated with any component of the study vaccines or diphtheria toxoid-containing vaccine, a serious, chronic disorder, known or suspected immunodeficiency, and acute illness.

Participants were randomized 1:1:1 to 1 of 3 vaccine groups. The Coadministration group received PCV20 in the right arm and BNT162b2 in the left arm at the same visit. The PCV20-only group received PCV20 in the right arm and saline in the left arm, and the BNT162b2-only group received BNT162b2 in the left arm and saline in the right arm. Randomization was stratified by prior pneumococcal vaccine status (naive or experienced) to ensure balanced assignments to the 3 groups within each stratum with no prespecified target numbers for any stratum. Blood samples for immunogenicity assessments were collected from all participants before vaccination (Visit 1) and approximately 1 month (21-35 days) later (Visit 2). Safety data were collected at Visit 2 and approximately 6 months after vaccination (Visit 3 telephone follow-up). Study staff preparing and administering PCV20, BNT162b2, and saline were unblinded, but all other study personnel and participants were blinded.

The study was conducted in accordance with all requirements and ethical principles derived in international guidelines including the Declaration of Helsinki, Council for International Organizations of Medical Sciences international ethical guidelines, and the International Council for Harmonisation Good Clinical Practice guidelines. The protocol and any amendments were approved by relevant institutional review boards and/or independent ethics committees before initiation of the study. Written informed consent was obtained from each participant (or their legally authorized representative) before any study-specific activity was conducted.

### Interventions

PCV20 0.5 mL containing capsular saccharides from pneumococcal serotypes 1, 3, 4, 5, 6A, 6B, 7F, 8, 9V, 10A, 11A, 12F, 14, 15B, 18C, 19A, 19F, 22F, 23F, and 33F, was injected intramuscularly into the right deltoid. BNT162b2 0.3 mL was injected intramuscularly into the left deltoid. Saline placebo was administered as a 0.3-mL dose injected intramuscularly into the left deltoid (in the PCV20-only group) or a 0.5-mL dose injected intramuscularly into the right deltoid (in the BNT162b2-only group).

### Objectives and Endpoints

The primary objective was to describe the safety of PCV20 and a booster dose of BNT162b2 when coadministered or administered alone. Endpoints evaluated included prompted local reactions (ie, redness, swelling, injection-site pain) at each injection site and prompted systemic events (ie, fever, fatigue, headache, chills, muscle pain, joint pain) recorded by participants each evening in an electronic diary for 10 and 7 days, respectively, after vaccination. Adverse events (AEs) and serious AEs (SAEs) were collected from before vaccination (ie, from signing the informed consent form) through 1 and 6 months, respectively, after vaccination.

Secondary objectives were to describe immune response to PCV20 using opsonophagocytic activity (OPA) geometric mean titers (GMTs) for the 20 serotypes 1 month after vaccination when PCV20 was administered alone or coadministered with BNT162b2, and to describe BNT162b2 responses to the SARS-COV-2 full length S-binding protein assessed as IgG geometric mean concentrations (GMCs) and GMFRs 1 month after vaccination.

Exploratory objectives for PCV20 responses included descriptive summaries of the percentage of participants with ≥4-fold rises in OPA titer and the OPA GMFRs for the 20 serotypes from before to 1 month after vaccination with PCV20, and the percentage of participants with pneumococcal titers greater than or equal to the lower limit of quantitation (≥LLOQ). Other exploratory objectives included descriptive summaries for BNT162b2 responses measured by SARS-COV-2 reference-strain neutralizing GMTs (50% virus neutralization) 1 month after BNT162b2 and GMFRs from before to 1 month after BNT162b2.

To support the secondary and exploratory objectives, serotype-specific OPA titers for the PCV20 serotypes were measured before and 1 month (21-35 days) after vaccination in the 2 groups that received PCV20. SARS-COV-2 full-length S-binding IgG concentrations and neutralizing titers against SARS-COV-2 wild type virus were measured before and 1 month after BNT162b2, the latter in a random subset of approximately 50% of participants in the groups that received BNT162b2. Additionally, N-binding antibodies were measured from blood samples collected at Visits 1 and 2 for serologic evidence of prior SARS-COV-2 infection in the Coadministration and BNT162b2-only groups.

### Statistical Analysis

Statistical analysis was descriptive, with no hypothesis testing. The target sample size was approximately 200 participants per group. Safety results were descriptively summarized in the safety population, which included all participants who received any study vaccination and had safety follow-up. Immunogenicity results were descriptively summarized for the evaluable immunogenicity population, which included participants who received the randomized vaccine(s), had ≥1 valid OPA titer or SARS-COV-2 full-length S-binding IgG concentration from a blood sample collected within a specified window 1 month after vaccination, and had no major protocol deviations. Participants with clinically documented SARS-COV-2 infection occurring between vaccination and 1 month after BNT162b2 vaccination were excluded from the analyses of SARS-CoV-2 full-length S-binding IgG concentrations or neutralizing titers.

Serotype-specific OPA GMTs, SARS-COV-2 full-length S-binding IgG GMCs, and SARS-COV-2 wild type neutralizing GMTs were calculated by exponentiating the mean logarithm of the corresponding assay results. GMFRs from before to approximately 1 month after vaccination were calculated as the mean of the difference of logarithmically transformed assay results (later minus earlier) and exponentiated back to the original units. Associated 2-sided 95% confidence intervals (Cls) were computed by exponentiating the confidence intervals using Student's t distribution for the means on the natural log scale.

A post hoc analysis using a linear regression model with terms for prior pneumococcal vaccine status, age, corresponding baseline OPA titers, sex, smoking status, body mass index group (<30 kg/m2 or ≥30 kg/m2), and vaccine group, was performed to compare serotype-specific OPA titers 1 month after vaccination in the Coadministration and PCV20-only groups. Similar post hoc analyses evaluated full-length S-binding IgG concentrations and SARS-COV-2 neutralizing antibody titers 1 month after vaccination in the Coadministration group compared with the BNT162b2-only group.

### Results

### Participants

A total of 570 participants were randomized, 559 (98.1%) of whom were vaccinated and comprised the safety population. The evaluable immunogenicity population included 549 participants (Coadministration group, n=184; PCV20-only group, n=182; BNT162b2-only group, n=183). Overall, 543 participants (95.3%) completed the study.

Demographic characteristics were generally similar between vaccine groups in the safety population (Table 1). More than half (56.9%) of participants were male, most were white and non Hispanic/Latino, and median age was 71 years. One-quarter of participants were pneumococcal vaccine-naive, 38.8% were previously vaccinated with PCV13 and PPSV23, 17.9% PCV13 only, and 16.6% received prior PPSV23 only, representing a variety of pneumococcal vaccination histories that were equally distributed among vaccine groups.

### Safety

The proportions of participants reporting local reactions within 10 days at each of the PCV20 and BNT162b2 injection sites were generally similar regardless of whether the vaccines were administered together or alone. Local reactions were generally mild or moderate in severity. Pain at the injection site was the most frequently reported local reaction for both PCV20 and BNT162b2 (in 60%-73% of participants). The percentage of participants reporting mild/moderate pain at the PCV20 injection site was lower in the PCV20-only group than at the PCV20 or BNT162b2 injection sites in the Coadministration group and BNT162b2-only group. The median duration of local reactions was 1.5 to 3 days for PCV20 and BNT162b2 injection sites in all groups.

Rates of systemic events were similar, varying by <5%, in the Coadministration group and the BNT162b2-only group, and lower in the PCV20-only group. Systemic events were generally mild to moderate in severity. Fatigue was the most frequently reported systemic event for all vaccine groups. Fever >38.9° C. was uncommon in all groups, and was only reported in 1 participant (0.5%) in the Coadministration group and 2 participants (1.1%) in the BNT162b2-only group. Systemic events generally resolved within median durations of 1 to 2 days.

Rates of AEs, SAEs, and severe AEs reported up to 1 month after vaccination were low and similar across all groups (Table 2). AEs considered related to vaccine by investigators were lymphadenopathy (axillary), injection site pain, diarrhea, and dizziness in the Coadministration group; and diarrhea in the BNT162b2-only group. No AEs led to discontinuation. The proportions of participants with any SAEs within 6 months after vaccination were low and similar across all vaccine groups, and were reported in 1 participant (0.5%) in the Coadministration group, which involved a death; 2 participants (1.1%) in the PCV20-only group; and 5 participants (2.7%) in the BNT162b2 group. These represented illnesses and medical conditions that may occur in populations of this age, with none considered vaccine-related.

### Immunogenicity

Immune Responses to PCV20 When Coadministered with a BNT162b2 Booster Dose

When coadministered with BNT162b2, PCV20 elicited robust immune responses at 1 month after vaccination to all 20 serotypes that were similar to that achieved when PCV20 was given alone (FIG. 7). The observed GMFRs from baseline to 1 month after PCV20 were generally similar for most serotypes when PCV20 was coadministered with BNT162b2 (2.5-24.5) or given alone (2.3-30.6). Percentages of participants with a ≥4-fold rise in OPA titers from baseline to 1 month after PCV20 vaccination were generally similar in the Coadministration (24.6-67.9%) and PCV20-only (22.7-71.0%) groups for most serotypes. The proportions of participants with OPA titers ≥LLOQ 1 month after vaccination with PCV20 were also similar (71.5-98.3% and 76.0-99.5% in the Coadministration and PCV20-only groups, respectively).

The post hoc analyses found the model-based OPA geometric mean ratios (GMRs) of the Coadministration group to the PCV20-only group 1 month after PCV20 ranged from 0.77 (serotypes 8, 19F, and 23F) to 1.11 (serotype 19A), with the lower bound of the GMR >0.5 for all 20 serotypes (FIG. 8). If a 2-fold noninferiority margin (lower bounds of the 2-sided 95% Cls for the model-based OPA GMRs >0.5) were applied to the results, OPA GMRs would have met noninferiority criteria for all 20 serotypes of the coadministration group compared to the PCV20-only group.

Immune Responses to BNT162b2 Booster Dose when Coadministered with PCV20

The BNT162b2 booster elicited robust immune IgG responses to the SARS-COV-2 full-length S-binding protein, which were similar whether BNT162b2 was coadministered with PCV20 or given alone (FIG. 9A). Observed GMFRs from before to 1 month after BNT162b2 booster were similar in the Coadministration and BNT162b2 only groups (35.5 and 39.0, respectively). Increases in reference strain neutralizing GMTs observed in the Coadministration and BNT162b2-only groups were also similar (FIG. 9B), with observed GMFRs from before to 1 month after the booster dose of BNT162b2 of 58.8-fold (Coadministration group) and 65.4-fold (BNT162b2 only group).

In the post hoc analysis of BNT162b2 responses, model-based GMRs of the Coadministration group to the BNT162b2-only group 1 month after the booster of BNT 162b2 were 1.06 (95% CI: 0.91, 1.23) for SARS-COV-2 full-length S-binding IgG, and 0.91 (95% CI: 0.71, 1.16) for SARS-COV-2 reference strain neutralizing titer. If a 1.5-fold noninferiority margin (lower bound of the 2-sided 95% CI for the model-based GMR >0.67) was applied to the results, comparisons of both endpoints for BNT162b2 responses in the coadministration group to the BNT162b2-only would have met noninferiority criteria.

### Discussion

We found that coadministration of PCV20 and BNT162b2 was well tolerated, with an overall safety profile similar to BNT162b2 administered alone. Robust immune responses were observed regardless of concomitant or separate administration of PCV20 and BNT162b2. These results provide support that coadministration of PCV20 with BNT162b2 does not result in clinically significant tolerability issues or interference with immune responses of either vaccine. By minimizing the number of required visits, coadministration of pneumococcal and COVID-19 vaccines in individuals recommended to receive both vaccines will potentially increase the uptake of both of these important vaccines targeting respiratory disease.

Many countries recommend COVID-19 vaccine booster doses [20], and PCV20 is recommended for older adults and those with factors that put them at increased risk for pneumococcal disease in the United States [22]. Vaccination against both diseases is important as both COVID-19 and pneumococcal disease cause significant morbidity and mortality, and are a burden on strained health care systems. Additionally, individuals with COVID-19 and pneumococcal disease together may be at increased risk of worse outcomes; in patients with invasive pneumococcal disease, COVID-19 co-infection was associated with a nearly 8-fold increased case-fatality in one study from the United Kingdom [23].

Although studies have evaluated coadministration of PCV13 and PCV20 with other vaccines, this is the first study examining coadministration with BNT162b2. The immunogenicity and safety of PCV13 has been demonstrated when coadministered with influenza vaccines [24-26], and a recently published study showed that PCV20 was well tolerated and elicited robust OPA responses to all 20 serotypes, with OPA GMTs meeting noninferiority criteria, regardless of coadministration with an influenza vaccine (Cannon, et al. Vaccine [submitted]).

The randomized, double-blind design is a study strength, as is the study population, which had already received 2 doses of COVID-19 vaccine to allow the evaluation of a booster dose, representing a large proportion of the general population. Additionally, this study was conducted in adults 65 years and older, a population well recognized for being significantly at risk for disease and severe complications of both COVID-19 and pneumococcal disease. Based on safety and immunogenicity data with PCV20 and COVID-19 vaccines, the findings in this study are expected to be extrapolated to younger adults. A potential limitation of this study is that the BNT162b2 mRNA vaccine was used in this study. Other SARS-COV-2 vaccine types and other manufacturers of mRNA COVID-19 vaccines exist, however, the lack of significant interaction with BNT162b2 and PCV20 should be applicable across vaccine types, analogous to coadministration findings of PCVs and influenza vaccines. Another limitation of this study was that it did not have predefined hypothesis tests because there were no prior data to use as a basis to power the study; the importance of generating data on coadministration took precedence. Nevertheless, the study permitted timely generation of adequate safety and immunogenicity data, and the results are presented for a post hoc analysis performed with the study data according to the rigor of formal hypothesis testing used for other PCV20 coadministration studies.

### Conclusions

These results support the safety and immunogenicity of coadministration of PCV20 and BNT162b2. The ability to administer these vaccines at the same visit may enhance uptake of both vaccines, and help protect against COVID-19 and pneumococcal disease.

TABLE 1
Participant demographics and clinical characteristics

| | | | BNT162b2 | |
|---|---|---|---|---|
| | Coadministration | PCV20 only | only | Total |
| Demographic | n = 187 | n = 187 | n = 185 | N = 559 |
| Sex | | | | |
| Male | 98 (52.4) | 112 (59.9) | 108 (58.4) | 318 (56.9) |
| Female | 89 (47.6) | 75 (40.1) | 77 (41.6) | 241 (43.1) |
| Race | | | | |
| White | 164 (87.7) | 172 (92.0) | 165 (89.2) | 501 (89.6) |
| Black or African American | 8 (4.3) | 7 (3.7) | 10 (5.4) | 25 (4.5) |
| Asian | 10 (5.3) | 5 (2.7) | 9 (4.9) | 24 (4.3) |
| Other^{a} | 5 (2.7) | 3 (1.6) | 1 (0.5) | 9 (1.6) |
| Ethnicity | | | | |
| Hispanic/Latino | 28 (15.0) | 26 (13.9) | 25 (13.5) | 79 (14.1) |
| Non-Hispanic/non-Latino | 159 (85.0) | 161 (86.1) | 160 (86.5) | 480 (85.9) |
| Age group | | | | |
| 65-69 years | 76 (40.6) | 69 (36.9) | 72 (38.9) | 217 (38.8) |
| 70-74 years | 73 (39.0) | 69 (36.9) | 59 (31.9) | 201 (36.0) |
| 75-79 years | 29 (15.5) | 34 (18.2) | 41 (22.2) | 104 (18.6) |
| ≥80 years | 9 (4.8) | 15 (8.0) | 13 (7.0) | 37 (6.6) |
| Mean age (SD), years | 71.0 (4.22) | 71.8 (4.94) | 71.8 (4.95) | 71.5 (4.72) |

All data are n (%) unless specified otherwise.
Data are for the safety population.
^{a}Includes American Indian or Alaska Native, multiracial, and not reported.
Abbreviations: PCV20, 20-valent pneumococcal conjugate vaccine; SD, standard deviation.

TABLE 2
Summary of AEs reported through 1 month^{a} after vaccination

| | Coadministration | | | PCV20 only | | BNT162b2 only | |
|---|---|---|---|---|---|---|---|
| | n = 187 | | | n = 187 | | n = 185 | |
| | n (%) | | (95% CI) | n (%) | (95% CI) | n (%) | (95% CI) |
| Any AE | 10 | (5.3) | (2.6, 9.6) | 8 (4.3) | (1.9, 8.3) | 12 (6.5) | (3.4, 11.1) |
| Any related AE^{b} | 4 | (2.1) | (0.6, 5.4) | 0 | (0, 2.0) | 1 (0.5) | (0, 3.0) |
| Any immediate AE^{c} | 1 | (0.5) | (0, 2.9) | 0 | (0, 2.0) | 0 | (0, 2.0) |
| Any severe AE^{d} | 1 | (0.5) | (0, 2.9) | 1 (0.5) | (0, 2.9) | 0 | (0, 2.0) |
| Any SAE^{e} | 1 | (0.5) | (0, 2.9) | 2 (1.1) | (0.1, 3.8) | 5 (2.7) | (0.9, 6.2) |

| | | | | | | |
|---|---|---|---|---|---|---|
| Related SAE | 0 | 0 | 0 | 0 | 0 | 0 |

Data are for the safety population.
^{a}SAEs were reported through 6 months after vaccination.
^{b}Coadministration group: lymphadenopathy (axillary), injection site pain, diarrhea, dizziness; BNT162b2-only group: diarrhea.
^{c}Dizziness.
^{d}Coadministration group: myalgia; PCV20-only group: unstable angina.
^{e}Coadministration group: duodenal perforation; PCV20-only group: unstable angina, glioblastoma; BNT162b2-only group: femur fracture, stage I breast cancer, small intestine carcinoma, metastases to liver, lung adenocarcinoma, acute kidney injury.
Abbreviations: AE, adverse event; CI, confidence interval; PCV20, 20-valent pneumococcal conjugate vaccine; SAE, serious adverse event.

### Example 4: Randomized trial to Evaluate the Safety, Tolerability, and Immunogenicity of a Booster (Third Dose) of BNT162b2 COVID-19 Vaccine Coadministered With 20-Valent Pneumococcal Conjugate Vaccine in Adults ≥65 Years Old

Background: Older adults are at increased risk of adverse outcomes from pneumococcal disease and COVID-19. Vaccination is an established strategy for preventing both illnesses. This study evaluated the safety and immunogenicity of coadministration of the 20-valent pneumococcal conjugate vaccine (PCV20) and a booster (third dose) of BNT162b2 COVID-19 vaccine.

Methods: This phase 3, randomized, double-blind, multicentre study included 570 participants aged ≥65 years randomized 1:1:1 to PCV20 and BNT162b2 coadministered, or PCV20 or BNT162b2 only (administered with saline for blinding). Primary safety endpoints included local reactions, systemic events, adverse events (AEs) and serious AEs (SAEs). Secondary objectives were immunogenicity of PCV20 and BNT162b2 when administered together or separately.

Results: Coadministration of PCV20 and BNT162b2 was well tolerated. Local reactions and systemic events were generally mild-moderate; injection-site pain and fatigue were the most frequent local and systemic events, respectively. AE and SAE rates were low and similar across groups. No AEs led to discontinuation; no SAEs were considered vaccination-related. Robust immune responses were observed, with opsonophagocytic activity geometric mean fold rises (GMFRs; from baseline to 1 month) of 2.5-24.5 and 2.3-30.6 across PCV20 serotypes in Coadministration and PCV20-only groups, respectively. GMFRs for full-length S-binding IgG of 35.5 and 39.0, and for neutralizing titres against SARS-COV-2-wild type virus of 58.8 and 65.4, were observed in the Coadministration and BNT162b2-only groups, respectively.

Conclusions: Safety and immunogenicity of coadministered PCV20 and BNT162b2 were similar to those of PCV20 or BNT162b2 administered alone, suggesting that the 2 vaccines may be coadministered.

### Methods

### Study Design and Participants

This phase 3, randomized, double-blind, multicentre study (ClinicalTrials.gov, NCT04887948), conducted at 25 sites in the United States between May 20-Dec. 8, 2021, evaluated the safety, tolerability, and immunogenicity of PCV20 when coadministered with a booster of BNT162b2 compared with individual administration. Eligible participants were ≥65 years old, healthy, or with stable pre-existing disease (not requiring significant change in therapy or hospitalization for worsening disease <6 weeks before enrolment). Participants had received 2 doses of 30 μg BNT162b2 in the pivotal efficacy study (C4591001), with the second dose given ≥6 months before vaccination in this study, and had not received a booster dose of any COVID-19 vaccine.

Individuals who had received pneumococcal vaccination <12 months prior were excluded, as were those with a history of microbiologically proven invasive disease caused by S pneumoniae or a previous clinical or microbiological diagnosis of COVID-19. Additional exclusion criteria were a history of severe adverse reaction associated with any component of the study vaccines or diphtheria toxoid-containing vaccine, a serious, chronic disorder, known or suspected immunodeficiency, and acute illness.

Participants were randomized 1:1:1 to 1 of 3 vaccine groups using centre-based randomization (Study design. In the Coadministration group, PCV20 and BNT162b2 vaccines were administered in one of each of the participant's arms. In the PCV20-only and BNT162b2-only groups, the respective active vaccine was given in one of the participant's arms with saline administered in the other arm. Local reactions and systemic events were captured by electronic diary for 10 and 7 days, respectively, after vaccination.). The Coadministration group received PCV20 in the right arm and BNT162b2 in the left arm at the same visit. The PCV20-only group received PCV20 in the right arm and saline in the left arm, and the BNT162b2-only group received BNT162b2 in the left arm and saline in the right arm. Randomization was stratified by prior pneumococcal vaccine status (naive or experienced) to ensure balanced assignments to the 3 groups within each stratum with no prespecified target numbers for any stratum. Blood samples for immunogenicity assessments were collected from all participants before vaccination (Visit 1) and approximately 1 month (21-35 days) later (Visit 2). Safety data were collected at Visit 2 and approximately 6 months after vaccination (Visit 3 telephone follow-up). Study staff preparing and administering PCV20, BNT162b2, and saline were unblinded, but all other study personnel and participants were blinded.

The study was conducted in accordance with all requirements and ethical principles derived in international guidelines including the Declaration of Helsinki, Council for International Organizations of Medical Sciences international ethical guidelines, and the International Council for Harmonisation Good Clinical Practice guidelines. The protocol and any amendments were approved by relevant institutional review boards and/or independent ethics committees before initiation of the study. Written informed consent was obtained from each participant (or their legally authorized representative) before any study-specific activity was conducted.

### Interventions

PCV20 0.5 mL (lot number 19-003657) containing capsular saccharides from pneumococcal serotypes 1, 3, 4, 5, 6A, 6B, 7F, 8, 9V, 10A, 11A, 12F, 14, 15B, 18C, 19A, 19F, 22F, 23F, and 33F, was injected intramuscularly into the right deltoid. BNT162b2 0.3 mL (lot number PA2094601) was injected intramuscularly into the left deltoid. Saline placebo was administered as a 0.3-mL dose injected intramuscularly into the left deltoid (in the PCV20-only group) or a 0.5-mL dose injected intramuscularly into the right deltoid (in the BNT162b2-only group).

### Objectives and Endpoints

The primary objective was to describe the safety of PCV20 and a booster dose of BNT162b2 when coadministered or administered alone. Endpoints evaluated included prompted local reactions (ie, redness, swelling, injection-site pain) at each injection site and prompted systemic events (ie, fever, fatigue, headache, chills, muscle pain, joint pain) recorded by participants each evening in an electronic diary for 10 and 7 days, respectively, after vaccination. Adverse events (AEs) and serious AEs (SAEs) were collected from before vaccination (ie, from signing the informed consent form) through 1 and 6 months, respectively, after vaccination.

Secondary objectives were to describe immune response to PCV20 using opsonophagocytic activity (OPA) geometric mean titres (GMTs) for the 20 serotypes 1 month after vaccination when PCV20 was administered alone or coadministered with BNT162b2, and to describe BNT162b2 responses to the SARS-COV-2 full length S-binding protein assessed as IgG geometric mean concentrations (GMCs) and GMFRs 1 month after vaccination.

Exploratory objectives for PCV20 responses included descriptive summaries of the percentage of participants with >4-fold rises in OPA titre and the OPA GMFRs for the 20 serotypes from before to 1 month after vaccination with PCV20, and the percentage of participants with pneumococcal titres greater than or equal to the lower limit of quantitation (≥LLOQ). Other exploratory objectives included descriptive summaries for BNT162b2 responses measured by SARS-COV-2 reference-strain neutralizing GMTs (50% virus neutralization) 1 month after BNT162b2 and GMFRs from before to 1 month after BNT162b2.

To support the secondary and exploratory objectives, serotype-specific OPA titres for the PCV20 serotypes were measured before and 1 month (21-35 days) after vaccination in the 2 groups that received PCV20. SARS-COV-2 full-length S-binding IgG concentrations and neutralizing titres against SARS-COV-2 wild type virus were measured before and 1 month after BNT162b2, the latter in a random subset of approximately 50% of participants in the groups that received BNT162b2. Additionally, N-binding antibodies were measured from blood samples collected at Visits 1 and 2 for serologic evidence of prior SARS-COV-2 infection in the Coadministration and BNT162b2-only groups.

### Statistical Analysis

Statistical analysis was descriptive, with no hypothesis testing. The target sample size was approximately 200 participants per group. Safety results were descriptively summarized in the safety population, which included all participants who received any study vaccination and had safety follow-up. Immunogenicity results were descriptively summarized for the evaluable immunogenicity population, which included participants who received the randomized vaccine(s), had ≥1 valid OPA titre or SARS-COV-2 full-length S-binding IgG concentration from a blood sample collected within a specified window 1 month after vaccination, and had no major protocol deviations. Participants with clinically documented SARS-COV-2 infection occurring between vaccination and 1 month after BNT162b2 vaccination were excluded from the analyses of SARS-COV-2 full-length S-binding IgG concentrations or neutralizing titres.

Serotype-specific OPA GMTs, SARS-COV-2 full-length S-binding IgG GMCs, and SARS-CoV-2 wild type neutralizing GMTs were calculated by exponentiating the mean logarithm of the corresponding assay results. GMFRs from before to approximately 1 month after vaccination were calculated as the mean of the difference of logarithmically transformed assay results (later minus earlier) and exponentiated back to the original units. Associated 2-sided 95% confidence intervals (CIs) were computed by exponentiating the confidence intervals using Student's t distribution for the means on the natural log scale.

A post hoc analysis using a linear regression model with terms for prior pneumococcal vaccine status, age, corresponding baseline OPA titres, sex, smoking status, body mass index group (<30 kg/m² or ≥30 kg/m²), and vaccine group, was performed to compare serotype-specific OPA titres 1 month after vaccination in the Coadministration and PCV20-only groups. Similar post hoc analyses evaluated full-length S-binding IgG concentrations and SARS-COV-2 neutralizing antibody titres 1 month after vaccination in the Coadministration group compared with the BNT162b2-only group.

### Results

### Participants

From 20 May 2021 to 8 Dec. 2021, 570 participants were randomized, 559 (98.1%) of whom were vaccinated and comprised the safety population. The evaluable immunogenicity population included 549 participants (Coadministration group, n=184; PCV20-only group, n=182; BNT162b2-only group, n=183). Overall, 543 participants (95.3%) completed the study.

Demographic characteristics were generally similar between vaccine groups in the safety population. More than half (56.9%) of participants were male, most were white and non-Hispanic/Latino, and median age was 71 years. One-quarter of participants were pneumococcal vaccine-naive, 38.8% were previously vaccinated with PCV13 and 23-valent pneumococcal polysaccharide vaccine (PPSV23), 17.9% PCV13 only, and 16.6% received prior PPSV23 only, representing a variety of pneumococcal vaccination histories that were equally distributed among vaccine groups.

### Immunogenicity

Immune Responses to PCV20 When Coadministered with a BNT16262 Booster Dose When coadministered with BNT162b2, PCV20 elicited robust immune responses at 1 month after vaccination to all 20 serotypes that were similar to that achieved when PCV20 was given alone (FIG. 7). The observed GMFRs from baseline to 1 month after PCV20 were generally similar for most serotypes when PCV20 was coadministered with BNT162b2 (2.5-24.5) or given alone (2.3-30.6). Percentages of participants with a ≥4-fold rise in OPA titres from baseline to 1 month after PCV20 vaccination were generally similar in the Coadministration (24.6-67.9%) and PCV20-only (22.7-71.0%) groups for most serotypes. The proportions of participants with OPA titres ≥LLOQ 1 month after vaccination with PCV20 were also similar (71.5-98.3% and 76.0-99.5% in the Coadministration and PCV20-only groups, respectively).

The post hoc analyses found the model-based OPA geometric mean ratios (GMRs) of the Coadministration group to the PCV20-only group 1 month after PCV20 ranged from 0.77 (serotypes 8, 19F, and 23F) to 1.11 (serotype 19A), with the lower bound of the GMR >0.5 for all 20 serotypes (FIG. 8). If a 2-fold noninferiority margin (lower bounds of the 2-sided 95% Cls for the model-based OPA GMRs >0.5) were applied to the results, OPA GMRs would have met noninferiority criteria for all 20 serotypes of the coadministration group compared to the PCV20-only group.

Immune Responses to BNT162b2 Booster Dose when Coadministered with PCV20

The BNT162b2 booster elicited robust immune IgG responses to the SARS-COV-2 full-length S-binding protein, which were similar whether BNT162b2 was coadministered with PCV20 or given alone (FIG. 9A). Observed GMFRs from before to 1 month after BNT162b2 booster were similar in the Coadministration and BNT162b2-only groups (35.5 and 39.0, respectively). Increases in reference strain neutralizing GMTs observed in the Coadministration and BNT162b2-only groups were also similar (FIG. 9B), with observed GMFRs from before to 1 month after the booster dose of BNT162b2 of 58.8-fold (Coadministration group) and 65.4-fold (BNT162b2-only group).

In the post hoc analysis of BNT162b2 responses, model-based GMRs of the Coadministration group to the BNT162b2-only group 1 month after the booster of BNT162b2 were 1.06 (95% CI: 0.91, 1.23) for SARS-COV-2 full-length S-binding IgG, and 0.91 (95% CI: 0.71, 1.16) for SARS-COV-2 reference strain neutralizing titre. If a 1.5-fold noninferiority margin (lower bound of the 2-sided 95% CI for the model-based GMR >0.67) was applied to the results, comparisons of both endpoints for BNT162b2 responses in the coadministration group to the BNT162b2-only would have met noninferiority criteria.

### Discussion

We found that coadministration of PCV20 and BNT162b2 was well tolerated, with an overall safety profile similar to BNT162b2 administered alone. Robust immune responses were observed regardless of concomitant or separate administration of PCV20 and BNT162b2. These results provide support that coadministration of PCV20 with BNT162b2 does not result in clinically significant tolerability issues or interference with immune responses of either vaccine. By minimizing the number of required visits, coadministration of pneumococcal and COVID-19 vaccines in individuals recommended to receive both vaccines will potentially increase the uptake of both of these important vaccines targeting respiratory disease.

Many countries recommend COVID-19 vaccine booster doses, and PCV20 is recommended for older adults and those with factors that put them at increased risk for pneumococcal disease in the United States. Vaccination against both diseases is important as both COVID-19 and pneumococcal disease cause significant morbidity and mortality, and are a burden on strained health care systems. Additionally, individuals with COVID-19 and pneumococcal disease together may be at increased risk of worse outcomes; in patients with invasive pneumococcal disease, COVID-19 co-infection was associated with a nearly 8-fold increased case-fatality in one study from the United Kingdom.

Although studies have evaluated coadministration of PCV13 and PCV20 with other vaccines, this is the first study examining coadministration with BNT162b2. The immunogenicity and safety of PCV13 has been demonstrated when coadministered with influenza vaccines, and a recently published study showed that PCV20 was well tolerated and elicited robust OPA responses to all 20 serotypes, with OPA GMTs meeting noninferiority criteria, regardless of coadministration with an influenza vaccine (Cannon, et al. Vaccine [submitted]).

The randomized, double-blind design is a study strength, as is the study population, which had already received 2 doses of COVID-19 vaccine to allow the evaluation of a booster dose, representing a large proportion of the general population. Additionally, this study was conducted in adults 65 years and older, a population well recognized for being significantly at risk for disease and severe complications of both COVID-19 and pneumococcal disease. Based on safety and immunogenicity data with PCV20 and COVID-19 vaccines, the findings in this study are expected to be extrapolated to younger adults. A potential limitation of this study is that the BNT162b2 mRNA vaccine was used in this study. Other SARS-COV-2 vaccine types and other manufacturers of mRNA COVID-19 vaccines exist, however, the lack of significant interaction with BNT162b2 and PCV20 should be applicable across vaccine types, analogous to coadministration findings of PCVs and influenza vaccines. Another limitation of this study was that it did not have predefined hypothesis tests because there were no prior data to use as a basis to power the study; the importance of generating data on coadministration took precedence. Nevertheless, the study permitted timely generation of adequate safety and immunogenicity data, and the results are presented for a post hoc analysis performed with the study data according to the rigor of formal hypothesis testing used for other PCV20 coadministration studies.

### Conclusions

These results support the safety and immunogenicity of coadministration of PCV20 and BNT162b2. The ability to administer these vaccines at the same visit may enhance uptake of both vaccines and help protect against COVID-19 and pneumococcal disease.

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference. Although the foregoing invention has been described in some detail by way of illustration and example for purposes of clarity of understanding, certain changes and modifications may be practiced within the scope of the appended claims.

### The Following Clauses Describe Additional Embodiments of the Invention

1. A method for eliciting an immunoprotective response in a human subject against S. pneumoniae and Severe Acute Respiratory Syndrome Coronavirus 2 (SARS-COV-2), the method comprising co-administering to the human subject an effective dose of a pneumococcal conjugate vaccine and of an mRNA vaccine against SARS-COV-2.
2. The method of clause 1 wherein said pneumococcal conjugate vaccine and said mRNA vaccine against SARS-COV-2 are administered concurrently or concomitantly.
3. The method of clause 1 wherein said pneumococcal conjugate vaccine and said mRNA vaccine against SARS-COV-2 are administered concurrently.
4. The method of clause 1 wherein said pneumococcal conjugate vaccine and said mRNA vaccine against SARS-COV-2 are administered concomitantly.
5. The method of any one of clauses 1 to 4 wherein one dose of each of the vaccines is administered.
6. The method of any one of clauses 1 to 4 wherein at least 2 doses of said mRNA vaccine against SARS-COV-2 is administered.
7. The method of any one of clauses 1 to 4 wherein at least 3 doses of said mRNA vaccine against SARS-COV-2 is administered.
8. The method of any one of clauses 1 to 4 wherein at least 4 doses of said mRNA vaccine against SARS-COV-2 is administered.
9. The method of any one of clauses 1 to 4 wherein 2 doses of said mRNA vaccine against SARS-COV-2 is administered.
10. The method of any one of clauses 1 to 4 wherein 3 doses of said mRNA vaccine against SARS-COV-2 is administered.
11. The method of any one of clauses 1 to 4 wherein 4 doses of said mRNA vaccine against SARS-COV-2 is administered.
12. The method of any one of clauses 6 to 11 wherein said doses of mRNA vaccine against SARS-COV-2 are separated by an interval of about 2 weeks to about 12 months.
13. The method of any one of clauses 1 to 12 wherein one dose of said pneumococcal conjugate vaccine is administered.
14. The method of any one of clauses 1 to 12 wherein at least 2 doses of said pneumococcal conjugate vaccine is administered.
15. The method of any one of clauses 1 to 12 wherein at least 3 doses of said pneumococcal conjugate vaccine is administered.
16. The method of any one of clauses 1 to 12 wherein at least 4 doses of said pneumococcal conjugate vaccine is administered.
17. The method of any one of clauses 1 to 12 wherein 2 doses of said pneumococcal conjugate vaccine is administered.
18. The method of any one of clauses 1 to 12 wherein 3 doses of said pneumococcal conjugate vaccine is administered.
19. The method of any one of clauses 1 to 12 wherein 4 doses of said pneumococcal conjugate vaccine is administered.
20. The method of any one of clauses 14 to 19 wherein said doses of pneumococcal conjugate vaccine are separated by an interval of about 2 weeks to about 12 months.
21. The method of any one of clauses 1 to 4 wherein 2 doses of mRNA vaccine against SARS-CoV-2 and one dose of pneumococcal conjugate vaccine are administered.
22. The method of clause 21 wherein said pneumococcal conjugate vaccine is co-administered with the first dose of mRNA vaccine against SARS-COV-2.
23. The method of clause 21 wherein said pneumococcal conjugate vaccine is co-administered with the second dose of mRNA vaccine against SARS-COV-2.
24. The method of clause 21 wherein said pneumococcal conjugate vaccine is concomitantly administered with the first dose of mRNA vaccine against SARS-COV-2.
25. The method of clause 21 wherein said pneumococcal conjugate vaccine is concomitantly administered with the second dose of mRNA vaccine against SARS-COV-2.
26. The method of clause 21 wherein said pneumococcal conjugate vaccine is concurrently administered with the first dose of mRNA vaccine against SARS-COV-2.
27. The method of clause 21 wherein said pneumococcal conjugate vaccine is c concurrently administered with the second dose of mRNA vaccine against SARS-COV-2.
28. The method of any one of clauses 1 to 4 wherein 3 doses of mRNA vaccine against SARS-CoV-2 and one dose of pneumococcal conjugate vaccine are administered.
29. The method of clause 28 wherein said pneumococcal conjugate vaccine is co-administered with the first dose of mRNA vaccine against SARS-COV-2.
30. The method of clause 28 wherein said pneumococcal conjugate vaccine is co-administered with the second dose of mRNA vaccine against SARS-COV-2.
31. The method of clause 28 wherein said pneumococcal conjugate vaccine is co-administered with the third dose of mRNA vaccine against SARS-COV-2.
32. The method of clause 28 wherein said pneumococcal conjugate vaccine is concurrently administered with the first dose of mRNA vaccine against SARS-COV-2.
33. The method of clause 28 wherein said pneumococcal conjugate vaccine is concurrently administered with the second dose of mRNA vaccine against SARS-COV-2.
34. The method of clause 28 wherein said pneumococcal conjugate vaccine is concurrently administered with the third dose of mRNA vaccine against SARS-COV-2.
35. The method of clause 28 wherein said pneumococcal conjugate vaccine is concomitantly administered with the first dose of mRNA vaccine against SARS-COV-2.
36. The method of clause 28 wherein said pneumococcal conjugate vaccine is concomitantly administered with the second dose of mRNA vaccine against SARS-COV-2.
37. The method of clause 28 wherein said pneumococcal conjugate vaccine is concomitantly administered with the third dose of mRNA vaccine against SARS-COV-2.
38. The method of any one of clauses 1 to 4 wherein 4 doses of mRNA vaccine against SARS-CoV-2 and one dose of pneumococcal conjugate vaccine are administered.
39. The method of clause 38 wherein said pneumococcal conjugate vaccine is co-administered with the first dose of mRNA vaccine against SARS-COV-2.
40. The method of clause 38 wherein said pneumococcal conjugate vaccine is co-administered with the second dose of mRNA vaccine against SARS-COV-2.
41. The method of clause 38 wherein said pneumococcal conjugate vaccine is co-administered with the third dose of mRNA vaccine against SARS-COV-2.
42. The method of clause 38 wherein said pneumococcal conjugate vaccine is co-administered with the fourth dose of mRNA vaccine against SARS-COV-2.
43. The method of clause 38 wherein said pneumococcal conjugate vaccine is concurrently administered with the first dose of mRNA vaccine against SARS-COV-2.
44. The method of clause 38 wherein said pneumococcal conjugate vaccine is concurrently administered with the second dose of mRNA vaccine against SARS-COV-2.
45. The method of clause 38 wherein said pneumococcal conjugate vaccine is concurrently administered with the third dose of mRNA vaccine against SARS-COV-2.
46. The method of clause 38 wherein said pneumococcal conjugate vaccine is concurrently administered with the fourth dose of mRNA vaccine against SARS-COV-2.
47. The method of clause 38 wherein said pneumococcal conjugate vaccine is concomitantly administered with the first dose of mRNA vaccine against SARS-COV-2.
48. The method of clause 38 wherein said pneumococcal conjugate vaccine is concomitantly administered with the second dose of mRNA vaccine against SARS-COV-2.
49. The method of clause 38 wherein said pneumococcal conjugate vaccine is concomitantly administered with the third dose of mRNA vaccine against SARS-COV-2.
50. The method of clause 38 wherein said pneumococcal conjugate vaccine is concomitantly administered with the fourth dose of mRNA vaccine against SARS-COV-2.
51. The method of any one of clauses 1 to 4 wherein 2 doses of mRNA vaccine against SARS-CoV-2 and 2 doses of pneumococcal conjugate vaccine are administered.
52. The method of any one of clauses 1 to 4 wherein 3 doses of mRNA vaccine against SARS-CoV-2 and 2 doses of pneumococcal conjugate vaccine are administered.
53. The method of any one of clauses 1 to 4 wherein 4 doses of mRNA vaccine against SARS-CoV-2 and 2 doses of pneumococcal conjugate vaccine are administered.
54. The method of any one of clauses 21 to 53 wherein the first 2 doses of mRNA vaccine against SARS-COV-2 are separated by an interval of about 2 weeks to about 6 months.
55. The method of any one of clauses 21 to 53 wherein the first 2 doses of mRNA vaccine against SARS-COV-2 are separated by an interval of about 2 weeks to about 2 months.
56. The method of any one of clauses 21 to 53 wherein the first 2 doses of mRNA vaccine against SARS-COV-2 are separated by an interval of about 2 weeks to about 6 weeks.
57. The method of any one of clauses 21 to 53 wherein the first 2 doses of mRNA vaccine against SARS-COV-2 are separated by an interval of about 2 weeks to about 4 months.
58. The method of any one of clauses 21 to 53 wherein the first 2 doses of mRNA vaccine against SARS-COV-2 are separated by an interval of about 3 weeks.
59. The method of any one of clauses 28 to 53 wherein the first 2 doses of mRNA vaccine against SARS-COV-2 are separated by an interval of about 2 weeks to about 6 months and the third dose is separated from the second dose by an interval of at least about 6 months.
60. The method of any one of clauses 28 to 53 wherein the first 2 doses of mRNA vaccine against SARS-COV-2 are separated by an interval of about 2 weeks to about 2 months and the third dose is separated from the second dose by an interval of at least about 6 months.
61. The method of any one of clauses 28 to 53 wherein the first 2 doses of mRNA vaccine against SARS-COV-2 are separated by an interval of about 2 weeks to about 6 weeks and the third dose is separated from the second dose by an interval of at least about 6 months.
62. The method of any one of clauses 28 to 53 wherein the first 2 doses of mRNA vaccine against SARS-COV-2 are separated by an interval of about 2 weeks to about 4 months and the third dose is separated from the second dose by an interval of at least about 6 months.
63. The method of any one of clauses 28 to 53 wherein the first 2 doses of mRNA vaccine against SARS-COV-2 are separated by an interval of about 3 weeks and the third dose is separated from the second dose by an interval of at least about 6 months.
64. The method of any one of clauses 28 to 53 wherein the first 2 doses of mRNA vaccine against SARS-COV-2 are separated by an interval of about 2 weeks to about 6 months and the third dose is separated from the second dose by an interval of at least about a year.
65. The method of any one of clauses 28 to 53 wherein the first 2 doses of mRNA vaccine against SARS-COV-2 are separated by an interval of about 2 weeks to about 2 months and the third dose is separated from the second dose by an interval of at least about a year.
66. The method of any one of clauses 28 to 53 wherein the first 2 doses of mRNA vaccine against SARS-COV-2 are separated by an interval of about 2 weeks to about 6 weeks and the third dose is separated from the second dose by an interval of at least about a year. 30)
67. The method of any one of clauses 28 to 53 wherein the first 2 doses of mRNA vaccine against SARS-COV-2 are separated by an interval of about 2 weeks to about 4 months and the third dose is separated from the second dose by an interval of at least about a year.
68. The method of any one of clauses 28 to 53 wherein the first 2 doses of mRNA vaccine against SARS-COV-2 are separated by an interval of about 3 weeks and the third dose is separated from the second dose by an interval of at least about a year.
69. The method of any one of clauses 1 to 4 wherein said human subject has already received at least one mRNA vaccine dose against SARS-COV-2 prior to said co-administration.
70. The method of clause 69 wherein said at least one mRNA vaccine dose against SARS-COV-2 has been administered at least about 3 weeks prior to said co-administration.
71. The method of clause 69 wherein said at least one mRNA vaccine dose against SARS-COV-2 has been administered at least about 2 months prior to said co-administration.
72. The method of clause 69 wherein said at least one mRNA vaccine dose against SARS-COV-2 has been administered at least about 6 months prior to said co-administration.
73. The method of clause 69 wherein said at least one mRNA vaccine dose against SARS-COV-2 has been administered at least about one year prior to said co-administration.
74. The method of clause 69 wherein said at least one mRNA vaccine dose against SARS-COV-2 has been administered at least about two years prior to said co-administration.
75. The method of any one of clauses 1 to 4 wherein said human subject has already received one mRNA vaccine dose against SARS-COV-2 prior to said co-administration.
76. The method of clause 75 wherein said one mRNA vaccine dose against SARS-COV-2 has been administered at least about 3 weeks prior to said co-administration.
77. The method of clause 75 wherein said one mRNA vaccine dose against SARS-COV-2 has been administered at least about 2 months prior to said co-administration.
78. The method of clause 75 wherein said one mRNA vaccine dose against SARS-COV-2 has been administered at least about 6 months prior to said co-administration.
79. The method of clause 75 wherein said one mRNA vaccine dose against SARS-COV-2 has been administered at least about one year prior to said co-administration.
80. The method of clause 75 wherein said one mRNA vaccine dose against SARS-COV-2 has been administered at least about two years prior to said co-administration.
81. The method of any one of clauses 1 to 4 wherein said human subject has already received at least two mRNA vaccine doses against SARS-COV-2 prior to said co-administration.
82. The method of clause 81 wherein the last of said at least two mRNA vaccine doses against SARS-COV-2 has been administered at least about 3 weeks prior to said co-administration.
83. The method of clause 81 wherein the last of said at least two mRNA vaccine doses against SARS-COV-2 has been administered at least about 2 months prior to said co-administration.
84. The method of clause 81 wherein the last of said at least two mRNA vaccine doses against SARS-COV-2 has been administered at least about 6 months prior to said co-administration.
85. The method of clause 81 wherein the last of said at least two mRNA vaccine doses against SARS-COV-2 has been administered at least about one year prior to said co-administration.
86. The method of clause 81 wherein the last of said at least two mRNA vaccine doses against SARS-COV-2 has been administered at least about two years prior to said co-administration.
87. The method of any one of clauses 1 to 4 wherein said human subject has already received two mRNA vaccine doses against SARS-COV-2 prior to said co-administration.
88. The method of clause 87 wherein the last of said two mRNA vaccine doses against SARS-CoV-2 has been administered at least about 3 weeks prior to said co-administration.
89. The method of clause 87 wherein the last of said two mRNA vaccine doses against SARS-CoV-2 has been administered at least about 2 months prior to said co-administration.
90. The method of clause 87 wherein the last of said two mRNA vaccine doses against SARS-CoV-2 has been administered at least about 6 months prior to said co-administration.
91. The method of clause 87 wherein the last of said two mRNA vaccine doses against SARS-CoV-2 has been administered at least about one year prior to said co-administration.
92. The method of clause 87 wherein the last of said two mRNA vaccine doses against SARS-CoV-2 has been administered at least about two years prior to said co-administration.
93. The method of any one of clauses 1 to 4 wherein said co-administration is a booster dose of said mRNA vaccine against SARS-COV-2.
94. A pneumococcal conjugate vaccine and an mRNA vaccine against SARS-COV-2 for use in a method for eliciting an immunoprotective response in a human subject against S. pneumoniae and Severe Acute Respiratory Syndrome Coronavirus 2 (SARS-COV-2), said method comprising co-administering to the human subject said vaccines.
95. The pneumococcal conjugate vaccine and mRNA vaccine against SARS-COV-2 for use of clause 94 wherein said pneumococcal conjugate vaccine and said mRNA vaccine against SARS-COV-2 are administered concurrently or concomitantly.
96. The pneumococcal conjugate vaccine and mRNA vaccine against SARS-COV-2 for use of clause 94 wherein said pneumococcal conjugate vaccine and said mRNA vaccine against SARS-COV-2 are administered concurrently.
97. The pneumococcal conjugate vaccine and mRNA vaccine against SARS-COV-2 for use of clause 94 wherein said pneumococcal conjugate vaccine and said mRNA vaccine against SARS-COV-2 are administered concomitantly.
98. The pneumococcal conjugate vaccine and mRNA vaccine against SARS-COV-2 for use of any one of clauses 94 to 97 wherein one dose of each of the vaccines is administered.
99. The pneumococcal conjugate vaccine and mRNA vaccine against SARS-COV-2 for use of any one of clauses 94 to 97 wherein at least 2 doses of said mRNA vaccine against SARS-CoV-2 is administered.
100. The pneumococcal conjugate vaccine and mRNA vaccine against SARS-COV-2 for use of any one of clauses 94 to 97 wherein at least 3 doses of said mRNA vaccine against SARS-CoV-2 is administered.
101. The pneumococcal conjugate vaccine and mRNA vaccine against SARS-COV-2 for use of any one of clauses 94 to 97 wherein at least 4 doses of said mRNA vaccine against SARS-CoV-2 is administered.
102. The pneumococcal conjugate vaccine and mRNA vaccine against SARS-COV-2 for use of any one of clauses 94 to 97 wherein 2 doses of said mRNA vaccine against SARS-COV-2 is administered.
103. The pneumococcal conjugate vaccine and mRNA vaccine against SARS-COV-2 for use of any one of clauses 94 to 97 wherein 3 doses of said mRNA vaccine against SARS-COV-2 is administered.
104. The pneumococcal conjugate vaccine and mRNA vaccine against SARS-COV-2 for use of any one of clauses 94 to 97 wherein 4 doses of said mRNA vaccine against SARS-COV-2 is administered.
105. The pneumococcal conjugate vaccine and mRNA vaccine against SARS-COV-2 for use of any one of clauses 99 to 104 wherein said doses of mRNA vaccine against SARS-COV-2 are separated by an interval of about 2 weeks to about 12 months.
106. The pneumococcal conjugate vaccine and mRNA vaccine against SARS-COV-2 for use of any one of clauses 94 to 105 wherein one dose of said pneumococcal conjugate vaccine is administered.
107. The pneumococcal conjugate vaccine and mRNA vaccine against SARS-COV-2 for use of any one of clauses 94 to 105 wherein at least 2 doses of said pneumococcal conjugate vaccine is administered.
108. The pneumococcal conjugate vaccine and mRNA vaccine against SARS-COV-2 for use of any one of clauses 94 to 105 wherein at least 3 doses of said pneumococcal conjugate vaccine is administered.
109. The pneumococcal conjugate vaccine and mRNA vaccine against SARS-COV-2 for use of any one of clauses 94 to 105 wherein at least 4 doses of said pneumococcal conjugate vaccine is administered.
110. The pneumococcal conjugate vaccine and mRNA vaccine against SARS-COV-2 for use of any one of clauses 94 to 105 wherein 2 doses of said pneumococcal conjugate vaccine is administered.
111. The pneumococcal conjugate vaccine and mRNA vaccine against SARS-COV-2 for use of any one of clauses 94 to 105 wherein 3 doses of said pneumococcal conjugate vaccine is administered.
112. The pneumococcal conjugate vaccine and mRNA vaccine against SARS-COV-2 for use of any one of clauses 94 to 105 wherein 4 doses of said pneumococcal conjugate vaccine is administered.
113. The pneumococcal conjugate vaccine and mRNA vaccine against SARS-COV-2 for use of any one of clauses 107 to 112 wherein said doses of pneumococcal conjugate vaccine are separated by an interval of about 2 weeks to about 12 months.
114. The pneumococcal conjugate vaccine and mRNA vaccine against SARS-COV-2 for use of any one of clauses 94 to 97 wherein 2 doses of mRNA vaccine against SARS-COV-2 and one dose of pneumococcal conjugate vaccine are administered.
115. The pneumococcal conjugate vaccine and mRNA vaccine against SARS-COV-2 for use of clause 114 wherein said pneumococcal conjugate vaccine is co-administered with the first dose of mRNA vaccine against SARS-COV-2.
116. The pneumococcal conjugate vaccine and mRNA vaccine against SARS-COV-2 for use of clause 114 wherein said pneumococcal conjugate vaccine is co-administered with the second dose of mRNA vaccine against SARS-COV-2.
117. The pneumococcal conjugate vaccine and mRNA vaccine against SARS-COV-2 for use of clause 114 wherein said pneumococcal conjugate vaccine is concomitantly administered with the first dose of mRNA vaccine against SARS-COV-2.
118. The pneumococcal conjugate vaccine and mRNA vaccine against SARS-COV-2 for use of clause 114 wherein said pneumococcal conjugate vaccine is concomitantly administered with the second dose of mRNA vaccine against SARS-COV-2.
119. The pneumococcal conjugate vaccine and mRNA vaccine against SARS-COV-2 for use of clause 114 wherein said pneumococcal conjugate vaccine is concurrently administered with the first dose of mRNA vaccine against SARS-COV-2.
120. The pneumococcal conjugate vaccine and mRNA vaccine against SARS-COV-2 for use of clause 114 wherein said pneumococcal conjugate vaccine is c concurrently administered with the second dose of mRNA vaccine against SARS-COV-2.
121. The pneumococcal conjugate vaccine and mRNA vaccine against SARS-COV-2 for use of any one of clauses 94 to 97 wherein 3 doses of mRNA vaccine against SARS-COV-2 and one dose of pneumococcal conjugate vaccine are administered.
122. The pneumococcal conjugate vaccine and mRNA vaccine against SARS-COV-2 for use of clause 121 wherein said pneumococcal conjugate vaccine is co-administered with the first dose of mRNA vaccine against SARS-COV-2.
123. The pneumococcal conjugate vaccine and mRNA vaccine against SARS-COV-2 for use of clause 121 wherein said pneumococcal conjugate vaccine is co-administered with the second dose of mRNA vaccine against SARS-COV-2.
124. The pneumococcal conjugate vaccine and mRNA vaccine against SARS-COV-2 for use of clause 121 wherein said pneumococcal conjugate vaccine is co-administered with the third dose of mRNA vaccine against SARS-COV-2.
125. The pneumococcal conjugate vaccine and mRNA vaccine against SARS-COV-2 for use of clause 121 wherein said pneumococcal conjugate vaccine is concurrently administered with the first dose of mRNA vaccine against SARS-COV-2.
126. The pneumococcal conjugate vaccine and mRNA vaccine against SARS-COV-2 for use of clause 121 wherein said pneumococcal conjugate vaccine is concurrently administered with the second dose of mRNA vaccine against SARS-COV-2.
127. The pneumococcal conjugate vaccine and mRNA vaccine against SARS-COV-2 for use of clause 121 wherein said pneumococcal conjugate vaccine is concurrently administered with the third dose of mRNA vaccine against SARS-COV-2.
128. The pneumococcal conjugate vaccine and mRNA vaccine against SARS-COV-2 for use of clause 121 wherein said pneumococcal conjugate vaccine is concomitantly administered with the first dose of mRNA vaccine against SARS-COV-2.
129. The pneumococcal conjugate vaccine and mRNA vaccine against SARS-COV-2 for use of clause 121 wherein said pneumococcal conjugate vaccine is concomitantly administered with the second dose of mRNA vaccine against SARS-COV-2.
130. The pneumococcal conjugate vaccine and mRNA vaccine against SARS-COV-2 for use of clause 121 wherein said pneumococcal conjugate vaccine is concomitantly administered with the third dose of mRNA vaccine against SARS-COV-2.
131. The pneumococcal conjugate vaccine and mRNA vaccine against SARS-COV-2 for use of any one of clauses 94 to 97 wherein 4 doses of mRNA vaccine against SARS-COV-2 and one dose of pneumococcal conjugate vaccine are administered.
132. The pneumococcal conjugate vaccine and mRNA vaccine against SARS-COV-2 for use of clause 131 wherein said pneumococcal conjugate vaccine is co-administered with the first dose of mRNA vaccine against SARS-COV-2.
133. The pneumococcal conjugate vaccine and mRNA vaccine against SARS-COV-2 for use of clause 131 wherein said pneumococcal conjugate vaccine is co-administered with the second dose of mRNA vaccine against SARS-COV-2.
134. The pneumococcal conjugate vaccine and mRNA vaccine against SARS-COV-2 for use of clause 131 wherein said pneumococcal conjugate vaccine is co-administered with the third dose of mRNA vaccine against SARS-COV-2.
135. The pneumococcal conjugate vaccine and mRNA vaccine against SARS-COV-2 for use of clause 131 wherein said pneumococcal conjugate vaccine is co-administered with the fourth dose of mRNA vaccine against SARS-COV-2.
136. The pneumococcal conjugate vaccine and mRNA vaccine against SARS-COV-2 for use of clause 131 wherein said pneumococcal conjugate vaccine is concurrently administered with the first dose of mRNA vaccine against SARS-COV-2.
137. The pneumococcal conjugate vaccine and mRNA vaccine against SARS-COV-2 for use of clause 131 wherein said pneumococcal conjugate vaccine is concurrently administered with the second dose of mRNA vaccine against SARS-COV-2.
138. The pneumococcal conjugate vaccine and mRNA vaccine against SARS-COV-2 for use of clause 131 wherein said pneumococcal conjugate vaccine is concurrently administered with the third dose of mRNA vaccine against SARS-COV-2.
139. The pneumococcal conjugate vaccine and mRNA vaccine against SARS-COV-2 for use of clause 131 wherein said pneumococcal conjugate vaccine is concurrently administered with the fourth dose of mRNA vaccine against SARS-COV-2.
140. The pneumococcal conjugate vaccine and mRNA vaccine against SARS-COV-2 for use of clause 131 wherein said pneumococcal conjugate vaccine is concomitantly administered with the first dose of mRNA vaccine against SARS-COV-2.
141. The pneumococcal conjugate vaccine and mRNA vaccine against SARS-COV-2 for use of clause 131 wherein said pneumococcal conjugate vaccine is concomitantly administered with the second dose of mRNA vaccine against SARS-COV-2.
142. The pneumococcal conjugate vaccine and mRNA vaccine against SARS-COV-2 for use of clause 131 wherein said pneumococcal conjugate vaccine is concomitantly administered with the third dose of mRNA vaccine against SARS-COV-2.
143. The pneumococcal conjugate vaccine and mRNA vaccine against SARS-COV-2 for use of clause 131 wherein said pneumococcal conjugate vaccine is concomitantly administered with the fourth dose of mRNA vaccine against SARS-COV-2.
144. The pneumococcal conjugate vaccine and mRNA vaccine against SARS-COV-2 for use of any one of clauses 94 to 97 wherein 2 doses of mRNA vaccine against SARS-COV-2 and 2 doses of pneumococcal conjugate vaccine are administered.
145. The pneumococcal conjugate vaccine and mRNA vaccine against SARS-COV-2 for use of any one of clauses 94 to 97 wherein 3 doses of mRNA vaccine against SARS-COV-2 and 2 doses of pneumococcal conjugate vaccine are administered.
146. The pneumococcal conjugate vaccine and mRNA vaccine against SARS-COV-2 for use of any one of clauses 94 to 97 wherein 4 doses of mRNA vaccine against SARS-COV-2 and 2 doses of pneumococcal conjugate vaccine are administered.
147. The pneumococcal conjugate vaccine and mRNA vaccine against SARS-COV-2 for use of any one of clauses 114 to 146 wherein the first 2 doses of mRNA vaccine against SARS-COV-2 are separated by an interval of about 2 weeks to about 6 months.
148. The pneumococcal conjugate vaccine and mRNA vaccine against SARS-COV-2 for use of any one of clauses 114 to 146 wherein the first 2 doses of mRNA vaccine against SARS-COV-2 are separated by an interval of about 2 weeks to about 2 months.
149. The pneumococcal conjugate vaccine and mRNA vaccine against SARS-COV-2 for use of any one of clauses 114 to 146 wherein the first 2 doses of mRNA vaccine against SARS-COV-2 are separated by an interval of about 2 weeks to about 6 weeks.
150. The pneumococcal conjugate vaccine and mRNA vaccine against SARS-COV-2 for use of any one of clauses 114 to 146 wherein the first 2 doses of mRNA vaccine against SARS-COV-2 are separated by an interval of about 2 weeks to about 4 months.
151. The pneumococcal conjugate vaccine and mRNA vaccine against SARS-COV-2 for use of any one of clauses 114 to 146 wherein the first 2 doses of mRNA vaccine against SARS-COV-2 are separated by an interval of about 3 weeks.
152. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 6 months and the third dose is separated from the second dose by an interval of at least about 6 months.
153. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 2 months and the third dose is separated from the second dose by an interval of at least about 6 months.
154. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 6 weeks and the third dose is separated from the second dose by an interval of at least about 6 months.
155. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 4 months and the third dose is separated from the second dose by an interval of at least about 6 months.
156. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 3 weeks and the third dose is separated from the second dose by an interval of at least about 6 months.
157. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 6 months and the third dose is separated from the second dose by an interval of at least about a year.
158. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 2 months and the third dose is separated from the second dose by an interval of at least about a year.
159. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 6 weeks and the third dose is separated from the second dose by an interval of at least about a year.
160. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 2 weeks to about 4 months and the third dose is separated from the second dose by an interval of at least about a year.
161. The pneumococcal conjugate vaccine and mRNA vaccine against SARS-COV-2 for use of any one of clauses 121 to 146 wherein the first 2 doses of mRNA vaccine against SARS-COV-2 are separated by an interval of about 3 weeks and the third dose is separated from the second dose by an interval of at least about a year.
162. The pneumococcal conjugate vaccine and mRNA vaccine against SARS-COV-2 for use of any one of clauses 94 to 97 wherein said human subject has already received at least one mRNA vaccine dose against SARS-COV-2 prior to said co-administration.
163. The pneumococcal conjugate vaccine and mRNA vaccine against SARS-COV-2 for use of clause 162 wherein said at least one mRNA vaccine dose against SARS-COV-2 has been administered at least about 3 weeks prior to said co-administration.
164. The pneumococcal conjugate vaccine and mRNA vaccine against SARS-COV-2 for use of clause 162 wherein said at least one mRNA vaccine dose against SARS-COV-2 has been administered at least about 2 months prior to said co-administration.
165. The pneumococcal conjugate vaccine and mRNA vaccine against SARS-COV-2 for use of clause 162 wherein said at least one mRNA vaccine dose against SARS-COV-2 has been administered at least about 6 months prior to said co-administration.
166. The pneumococcal conjugate vaccine and mRNA vaccine against SARS-COV-2 for use of clause 162 wherein said at least one mRNA vaccine dose against SARS-COV-2 has been administered at least about one year prior to said co-administration.
167. The pneumococcal conjugate vaccine and mRNA vaccine against SARS-COV-2 for use of clause 162 wherein said at least one mRNA vaccine dose against SARS-COV-2 has been administered at least about two years prior to said co-administration.
168. The pneumococcal conjugate vaccine and mRNA vaccine against SARS-COV-2 for use of any one of clauses 94 to 97 wherein said human subject has already received one mRNA vaccine dose against SARS-COV-2 prior to said co-administration.
169. The pneumococcal conjugate vaccine and mRNA vaccine against SARS-COV-2 for use of clause 168 wherein said one mRNA vaccine dose against SARS-COV-2 has been administered at least about 3 weeks prior to said co-administration.
170. The pneumococcal conjugate vaccine and mRNA vaccine against SARS-COV-2 for use of clause 168 wherein said one mRNA vaccine dose against SARS-COV-2 has been administered at least about 2 months prior to said co-administration.
171. The pneumococcal conjugate vaccine and mRNA vaccine against SARS-COV-2 for use of clause 168 wherein said one mRNA vaccine dose against SARS-COV-2 has been administered at least about 6 months prior to said co-administration.
172. The pneumococcal conjugate vaccine and mRNA vaccine against SARS-COV-2 for use of clause 168 wherein said one mRNA vaccine dose against SARS-COV-2 has been administered at least about one year prior to said co-administration.
173. The pneumococcal conjugate vaccine and mRNA vaccine against SARS-COV-2 for use of clause 168 wherein said one mRNA vaccine dose against SARS-COV-2 has been administered at least about two years prior to said co-administration.
174. The pneumococcal conjugate vaccine and mRNA vaccine against SARS-COV-2 for use of any one of clauses 94 to 97 wherein said human subject has already received at least two mRNA vaccine doses against SARS-COV-2 prior to said co-administration.
175. The pneumococcal conjugate vaccine and mRNA vaccine against SARS-COV-2 for use of clause 174 wherein the last of said at least two mRNA vaccine doses against SARS-COV-2 has been administered at least about 3 weeks prior to said co-administration.
176. The pneumococcal conjugate vaccine and mRNA vaccine against SARS-COV-2 for use of clause 174 wherein the last of said at least two mRNA vaccine doses against SARS-COV-2 has been administered at least about 2 months prior to said co-administration.
177. The pneumococcal conjugate vaccine and mRNA vaccine against SARS-COV-2 for use of clause 174 wherein the last of said at least two mRNA vaccine doses against SARS-COV-2 has been administered at least about 6 months prior to said co-administration.
178. The pneumococcal conjugate vaccine and mRNA vaccine against SARS-COV-2 for use of clause 174 wherein the last of said at least two mRNA vaccine doses against SARS-COV-2 has been administered at least about one year prior to said co-administration.
179. The pneumococcal conjugate vaccine and mRNA vaccine against SARS-COV-2 for use of clause 174 wherein the last of said at least two mRNA vaccine doses against SARS-COV-2 has been administered at least about two years prior to said co-administration.
180. The pneumococcal conjugate vaccine and mRNA vaccine against SARS-COV-2 for use of any one of clauses 94 to 97 wherein said human subject has already received two mRNA vaccine doses against SARS-COV-2 prior to said co-administration.
181. The pneumococcal conjugate vaccine and mRNA vaccine against SARS-COV-2 for use of clause 180 wherein the last of said two mRNA vaccine doses against SARS-COV-2 has been administered at least about 3 weeks prior to said co-administration.
182. The pneumococcal conjugate vaccine and mRNA vaccine against SARS-COV-2 for use of clause 180 wherein the last of said two mRNA vaccine doses against SARS-COV-2 has been administered at least about 2 months prior to said co-administration.
183. The pneumococcal conjugate vaccine and mRNA vaccine against SARS-COV-2 for use of clause 180 wherein the last of said two mRNA vaccine doses against SARS-COV-2 has been administered at least about 6 months prior to said co-administration.
184. The pneumococcal conjugate vaccine and mRNA vaccine against SARS-COV-2 for use of clause 180 wherein the last of said two mRNA vaccine doses against SARS-COV-2 has been administered at least about one year prior to said co-administration.
185. The pneumococcal conjugate vaccine and mRNA vaccine against SARS-COV-2 for use of clause 180 wherein the last of said two mRNA vaccine doses against SARS-COV-2 has been administered at least about two years prior to said co-administration.
186. The pneumococcal conjugate vaccine and mRNA vaccine against SARS-COV-2 for use of any one of clauses 94 to 97 wherein said co-administration is a booster dose of said mRNA vaccine against SARS-COV-2.
187. Use of the co-administration of a pneumococcal conjugate vaccine and of an mRNA vaccine against SARS-COV-2 as a booster dose of an mRNA vaccine against SARS-COV-2.
188. Use of the co-administration of a pneumococcal conjugate vaccine and of an mRNA vaccine against SARS-COV-2 as a booster dose of said mRNA vaccine against SARS-COV-2.
189. Use of the co-administration of a pneumococcal conjugate vaccine and of an mRNA vaccine against SARS-COV-2 for boosting an mRNA vaccine against SARS-COV-2.
190. Use of the co-administration of a pneumococcal conjugate vaccine and of an mRNA vaccine against SARS-COV-2 for boosting said mRNA vaccine against SARS-COV-2.
191. The co-administration of a pneumococcal conjugate vaccine and of an mRNA vaccine against SARS-COV-2 for use as a booster dose of an mRNA vaccine against SARS-COV-2.
192. The co-administration of a pneumococcal conjugate vaccine and of an mRNA vaccine against SARS-COV-2 for use as a booster dose of said mRNA vaccine against SARS-COV-2.
193. The co-administration of a pneumococcal conjugate vaccine and of an mRNA vaccine against SARS-COV-2 for use in a method of boostering an mRNA vaccine against SARS-CoV-2.
194. The co-administration of a pneumococcal conjugate vaccine and of an mRNA vaccine against SARS-COV-2 for use in a method of boostering said mRNA vaccine against SARS-CoV-2.
195. The method of any one of clauses 1 to 194 wherein said pneumococcal conjugate vaccine is a 13-valent pneumococcal vaccine.
196. The method of any one of clauses 1 to 194 wherein said pneumococcal conjugate vaccine is a 14-valent pneumococcal vaccine.
197. The method of any one of clauses 1 to 194 wherein said pneumococcal conjugate vaccine is a 15-valent pneumococcal vaccine.
198. The method of any one of clauses 1 to 194 wherein said pneumococcal conjugate vaccine is a 16-valent pneumococcal vaccine.
199. The method of any one of clauses 1 to 194 wherein said pneumococcal conjugate vaccine is a 20-valent pneumococcal vaccine 200. The method of any one of clauses 1 to 199 wherein the glycoconjugates from S. pneumoniae are all individually conjugated to CRM197.
201. The method of any one of clauses 1 to 194 wherein said pneumococcal conjugate vaccine comprises 13 glycoconjugates from a Streptococcus pneumoniae serotype selected from the group consisting of serotypes 1, 3, 4, 5, 6A, 6B, 7F, 8, 9V, 10A, 11A, 12F, 14, 15B, 18C, 19A, 19F, 23F, 22F and 33F.
202. The method clause 201 wherein said glycoconjugates are all individually conjugated to CRM197.
203. The method of any one of clauses 1 to 194 wherein said pneumococcal conjugate vaccine is a 13-valent pneumococcal conjugate vaccine wherein said 13 conjugates consists of glycoconjugates from S. pneumoniae serotypes 1, 3, 4, 5, 6A, 6B, 7F, 9V, 14, 18C, 19A, 19F and 23F.
204. The method clause 203 wherein said glycoconjugates are all individually conjugated to CRM197.
205. The method of any one of clauses 1 to 194 wherein said pneumococcal conjugate vaccine is a 15-valent pneumococcal conjugate vaccine wherein said 15 conjugates consists of glycoconjugates from S. pneumoniae serotypes 1, 3, 4, 5, 6A, 6B, 7F, 9V, 14, 18C, 19A, 19F, 23F, 22F and 33F.
206. The method clause 205 wherein said glycoconjugates are all individually conjugated to CRM197.
207. The method of any one of clauses 1 to 194 wherein said pneumococcal conjugate vaccine is a 20-valent pneumococcal conjugate vaccine wherein said 20 conjugates consists of glycoconjugates from S. pneumoniae serotypes 1, 3, 4, 5, 6A, 6B, 7F, 8, 9V, 10A, 11A, 12F, 14, 15B, 18C, 19A, 19F, 23F, 22F and 33F.
208. The method clause 207 wherein said glycoconjugates are all individually conjugated to CRM197.
209. The method of any one of clauses 1 to 194 wherein said pneumococcal conjugate vaccine is PREVNAR 13®/
210. The method of any one of clauses 1 to 194 wherein said pneumococcal conjugate vaccine is V114.
211. The method of any one of clauses 1 to 194 wherein said pneumococcal conjugate vaccine is 20vPnC.
212. The method of any one of clauses 1 to 211 wherein said mRNA vaccine against SARS-CoV-2 comprises nucleoside-modified mRNA.
213. The method of any one of clauses 1 to 211 wherein said mRNA vaccine is BNT162b2 (Comirnaty®).
214. The method of any one of clauses 1 to 212 wherein said mRNA vaccine against SARS-CoV-2 comprises a sequence having residues 1-102 of SEQ ID NO: 1 and residues 103-4284 of SEQ ID NO: 1, wherein the sequence for the SARS-COV-2 antigen of SEQ ID NO: 1 is replaced with SARS-COV-2 antigen of a variant strain.
215. The method of any one of clauses 1 to 212 wherein said mRNA vaccine is vaccine “mRNA-1273”.
216. The method of any one of clauses 1 to 212 wherein said mRNA vaccine against SARS-COV-2 comprises a mRNA which includes a first region of linked nucleosides encoding a SARS-COV-2 antigen (e.g., S protein), a first flanking region located at the 5 ′-terminus of the first region (e.g., a 5′-UTR), a second flanking region located at the 3 ′-terminus of the first region (e.g., a 3′-UTR), at least one 5 ′-cap region, and a 3′-stabilizing region.
217. The method of any one of clauses 1 to 212 wherein said mRNA vaccine against SARS-COV-2 comprises a mRNA which includes a first region of linked nucleosides encoding a a mutated viral spike(S) glycoprotein of SARS-COV-2, a first flanking region located at the 5 ′-terminus of the first region (e.g., a 5′-UTR), a second flanking region located at the 3 ′-terminus of the first region (e.g., a 3′-UTR), at least one 5 ′-cap region, and a 3′-stabilizing region.
218. A method of treating a respiratory viral infection in a human, comprising administering a first composition comporising a compound represented by Formula (I) as follows

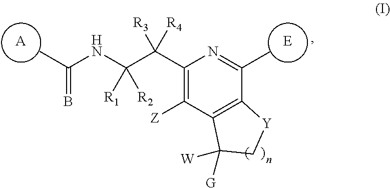

or a pharmaceutically acceptable salt thereof, wherein: A is selected from the group consisting of: 1) optionally substituted aryl; and 2) optionally substituted heteroaryl; B is O or S; R1 and R2 are each independently selected from the group consisting of: 1) hydrogen; 2) fluorine; and 3) optionally substituted-C1-C6 alkyl; alternatively, R1 and R2 are taken together with the carbon atom to which they are attached to form an optionally substituted 3- to 6-membered ring; Z is selected from the group consisting of: 1) hydrogen; 2) halogen; 3) hydroxy; 4) cyano; 5) nitro; 6) optionally substituted-C1-C6 alkoxy; and 7) optionally substituted-C1-C6 alkyl; W is selected from the group consisting of: 1) hydrogen; 2) optionally substituted-C1-C6 alkoxy; 3) optionally substituted-C1-C6 alkyl; and 4) optionally substituted-C3-C6 cycloalkyl; G is selected from the group consisting of: 1) —C(O)OR12; 2) —C(O)NR11R12; 3) optionally substituted-C1-C6 alkyl-CN; 4) optionally substituted-C1-C6 alkyl-C(O)NR11R12; 5) optionally substituted-C1-C6 alkyl-C(O)NR11S(O)2R12; 6) optionally substituted-C1-C6 alkyl-OC(O)NR11R12; 7) optionally substituted-C1-C6 alkyl-NHR13; 8) optionally substituted-C1-C6 alkyl-NHC(O)R13; and 9) —C(O)NR11S(O)2R12; n is 1, 2 or 3; Y is O, S, S(O)2, or NR14; E is selected from the group consisting of: 1) optionally substituted aryl; 2) optionally substituted heteroaryl; 3) optionally substituted 3- to 8-membered heterocyclic, and 4) optionally substituted alkynyl; R3 is hydroxy or fluorine; R4 is selected from the group consisting of: 1) hydrogen; 2) optionally substituted-C1-C6 alkyl; 3) optionally substituted-C3-C8 cycloalkyl; and 4) optionally substituted 3- to 8-membered heterocyclic; R11 at each occurrence is independently selected from the group consisting of: 1) hydrogen; 2) optionally substituted-C1-C8-alkyl; 3) optionally substituted-C3-C8-cycloalkyl; 4) optionally substituted 3- to 8-membered heterocyclic; 5) optionally substituted aryl; 6) optionally substituted arylalkyl; 7) optionally substituted heteroaryl; and 8) optionally substituted heteroarylalkyl; R12 at each occurrence is independently selected from the group consisting of: 1) hydrogen; 2) optionally substituted-C1-C8-alkyl; 3) optionally substituted-C3-C8-cycloalkyl; 4) optionally substituted 3- to 8-membered heterocyclic; 5) optionally substituted aryl; 6) optionally substituted arylalkyl; 7) optionally substituted heteroaryl; and 8) optionally substituted heteroarylalkyl; alternatively, Rn and R12 are taken together with the nitrogen atom to which they are attached to form a 3- to 12-membered heterocyclic ring; R13 at each occurrence is independently selected from the group consisting of: 1) Optionally substituted-C1-C8 alkyl; 2) Optionally substituted-C3-C8 cycloalkyl; 3) Optionally substituted 3- to 8-membered heterocyclic; 4) Optionally substituted aryl; 5) Optionally substituted arylalkyl; 6) Optionally substituted heteroaryl; and 7) Optionally substituted heteroarylalkyl; and R14 is selected from: 1) hydrogen; 2) optionally substituted-C1-C8-alkyl; and 3) optionally substituted-C3-C5-cycloalkyl; and a second composition, wherein the second composition is an immunogenic composition comprising an mRNA encoding an antigen encapsulated in a lipid nanoparticle.

1. A method for eliciting an immunoprotective response in a human subject against S. pneumoniae and Severe Acute Respiratory Syndrome Coronavirus 2 (SARS-COV-2), the method comprising co-administering to the human subject an effective dose of a pneumococcal conjugate vaccine and of an mRNA vaccine against SARS-COV-2, wherein at least two doses of the mRNA vaccine against SARS-COV-2 have been administered to the human.

2. The method of claim 1 wherein said pneumococcal conjugate vaccine and said mRNA vaccine against SARS-COV-2 are administered concurrently or concomitantly.

3. The method of any one of claims 1 to 2, wherein one dose of each of the vaccines is administered.

4. The method of any one of claims 1 to 3, wherein at least 2 doses of said pneumococcal conjugate vaccine is administered.

5. The method of any one of claims 1 to 4, wherein at least 3 doses of said pneumococcal conjugate vaccine is administered.

6. The method of any one of claims 4 to 5, wherein said doses of pneumococcal conjugate vaccine are separated by an interval of about 2 weeks to about 12 months.

7. The method of any one of claims 1 to 6, wherein 2 doses of mRNA vaccine against SARS-CoV-2 and one dose of pneumococcal conjugate vaccine are administered.

8. The method of any one of claims 1 to 7, wherein said pneumococcal conjugate vaccine is a 13-valent pneumococcal vaccine.

9. The method of any one of claims 1 to 7, wherein said pneumococcal conjugate vaccine is a 14-valent pneumococcal vaccine.

10. The method of any one of claims 1 to 7, wherein said pneumococcal conjugate vaccine is a 15-valent pneumococcal vaccine.

11. The method of any one of claims 1 to 7, wherein said pneumococcal conjugate vaccine is a 16-valent pneumococcal vaccine.

12. The method of any one of claims 1 to 7, wherein said pneumococcal conjugate vaccine is a 20-valent pneumococcal vaccine

13. The method of any one of claims 1 to 12, wherein the glycoconjugates from S. pneumoniae are all individually conjugated to CRM₁₉₇.

14. The method of any one of claims 1 to 8, wherein said pneumococcal conjugate vaccine comprises 13 glycoconjugates from a Streptococcus pneumoniae serotype selected from the group consisting of serotypes 1, 3, 4, 5, 6A, 6B, 7F, 8, 9V, 10A, 11A, 12F, 14, 15B, 18C, 19A, 19F, 23F, 22F and 33F.

15. The method of claim 14, wherein said glycoconjugates are all individually conjugated to CRM197.

16. The method of any one of claims 1 to 8, wherein said pneumococcal conjugate vaccine is a 13-valent pneumococcal conjugate vaccine wherein said 13 conjugates consists of glycoconjugates from S. pneumoniae serotypes 1, 3, 4, 5, 6A, 6B, 7F, 9V, 14, 18C, 19A, 19F and 23F.

17. The method of claim 16, wherein said glycoconjugates are all individually conjugated to CRM197.

18. The method of any one of claims 1 to 7, wherein said pneumococcal conjugate vaccine is a 15-valent pneumococcal conjugate vaccine wherein said 15 conjugates consists of glycoconjugates from S. pneumoniae serotypes 1, 3, 4, 5, 6A, 6B, 7F, 9V, 14, 18C, 19A, 19F, 23F, 22F and 33F.

19. The method of claim 18, wherein said glycoconjugates are all individually conjugated to CRM197.

20. The method of any one of claims 1 to 7, wherein said pneumococcal conjugate vaccine is a 20-valent pneumococcal conjugate vaccine wherein said 20 conjugates consists of glycoconjugates from S. pneumoniae serotypes 1, 3, 4, 5, 6A, 6B, 7F, 8, 9V, 10A, 11A, 12F, 14, 15B, 18C, 19A, 19F, 23F, 22F and 33F.

21. The method of claim 20, wherein said glycoconjugates are all individually conjugated to CRM197.

22. The method of any one of claims 1 to 7, wherein said pneumococcal conjugate vaccine is PREVNAR 13®.

23. The method of any one of claims 1 to 7, wherein said pneumococcal conjugate vaccine is V114.

24. The method of any one of claims 1 to 7, wherein said pneumococcal conjugate vaccine is 20vPnC.

25. The method of any one of claims 1 to 24, wherein said mRNA vaccine against SARS-CoV-2 comprises nucleoside-modified mRNA.

26. The method of any one of claims 1 to 25, wherein said mRNA vaccine is BNT162b2 (Comimaty®).
